(19) United States
(12) Patent Application Publication
(10) Pub. No.: US 2008/0045513 A1
(43) Pub. Date: Feb. 21, 2008

(54) COMBINATION FAAH INHIBITOR AND ANALGESIC, ANTI-INFLAMMATORY OR ANTI-PYRETIC AGENT

(75) Inventor: Olivier DASSE, Foothill Ranch, CA (US)

Correspondence Address: ORGANON USA, INC.;PATENT DEPARTMENT, 56 LIVINGSTON AVENUE, ROSELAND, NJ, 07068, US

(73) Assignee: N.V. Organon, Oss (NL)

(21) Appl. No.: 11/772,597

(22) Filed: Jul. 2, 2007

Related U.S. Application Data
(60) Provisional application No. 60/822,877, filed on Aug. 18, 2006.

(51) Int. Cl. A61K 31/27; A61K 31/5415; A61P 29/00; C07C 271/40; C07C 271/58; C07D 279/16

(52) U.S. Cl. 514/222.2; 514/484; 514/490; 544/49; 560/157; 560/163

(57) ABSTRACT

Pharmacological inhibition of fatty acid amide hydrolase (FAAH) activity leads to increased levels of fatty acid amides. Esters of alkylcarbamic acids are disclosed that are inhibitors of FAAH activity. Compounds disclosed herein inhibit FAAH activity and further provide an analgesic, anti-inflammatory, or anti-pyretic agent. Described herein is a process for the preparation of esters of alkylcarbamic acid compounds, compositions that include them, and methods of use thereof.

### CROSS-REFERENCE TO RELATED APPLICATIONS

This application claims priority to the U.S. Provisional Application No.60/822,877, entitled “COMBINATION FAAH INHIBITOR AND ANALGESIC, ANTI-INFLAMMATORY OR ANTI-PYRETIC AGENT,” filed Aug. 18, 2006, which is incorporated by reference in its entirety.

### FIELD OF THE INVENTION

Described herein are compounds, methods of making such compounds, pharmaceutical compositions and medicaments containing such compounds, and methods of using such compounds and compositions to inhibit the activity of fatty acid amide hydrolase (FAAH) and act as an analgesic, anti-inflammatory, and/or anti-pyretic.

### BACKGROUND OF THE INVENTION

Fatty acid amide hydrolase (FAAH) is an enzyme that hydrolyzes the fatty acid amide (FAA) family of endogenous signaling lipids. General classes of FAAs include the N-acylethanolamines (NAEs) and fatty acid primary amides (FAPAs). Examples of NAEs include anandamide (AEA), palmitoylethanolamide (PEA) and oleoylethanolamide (OEA). Pharmacological inhibition of FAAH activity results in increases in the levels of these fatty acid amides.

### SUMMARY OF THE INVENTION

Compounds, compositions and methods for inhibiting the activity of fatty acid amide hydrolase (FAAH) are provided. Among the compounds provided herein are compounds that are inhibitors of fatty acid amide hydrolase (FAAH). Processes for the preparation of compounds that inhibit the activity of fatty acid amide hydrolase, compositions that include the compounds, as well as methods of use thereof are provided. Also provided are compounds, process for preparing such compounds, and formulations of such compounds that upon inhibition of FAAH can further provide an agent that is an analgesic, anti-inflammatory, and/or anti-pyretic.

In one aspect are compounds of Formula (I): 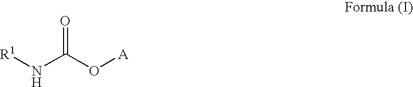
wherein:

R¹ is an optionally substituted group selected from among C₁-C₆ alkyl, C₃-C₉ cycloalkyl, and —C₁-C₄alkyl-(C₃-C₉cycloalkyl);

O-A is a deprotonated form of a hydroxy-containing compound selected from acetaminophen, propofol, an analgesic agent, an anti-inflammatory agent, an anti-pyretic agent, an NSAID, a metabolite of an analgesic agent, a metabolite of an anti-inflammatory agent, a metabolite of an anti-pyretic agent, and an NSAID metabolite; and pharmaceutically acceptable salts, pharmaceutically acceptable N-oxides, pharmaceutically active metabolites, pharmaceutically acceptable prodrugs, and pharmaceutically acceptable solvates thereof.

The compound of claim 1, wherein O-A is the deprotonated form of acetaminophen.

The compound of claim 1, wherein the compound of Formula (I) has a structure selected from:

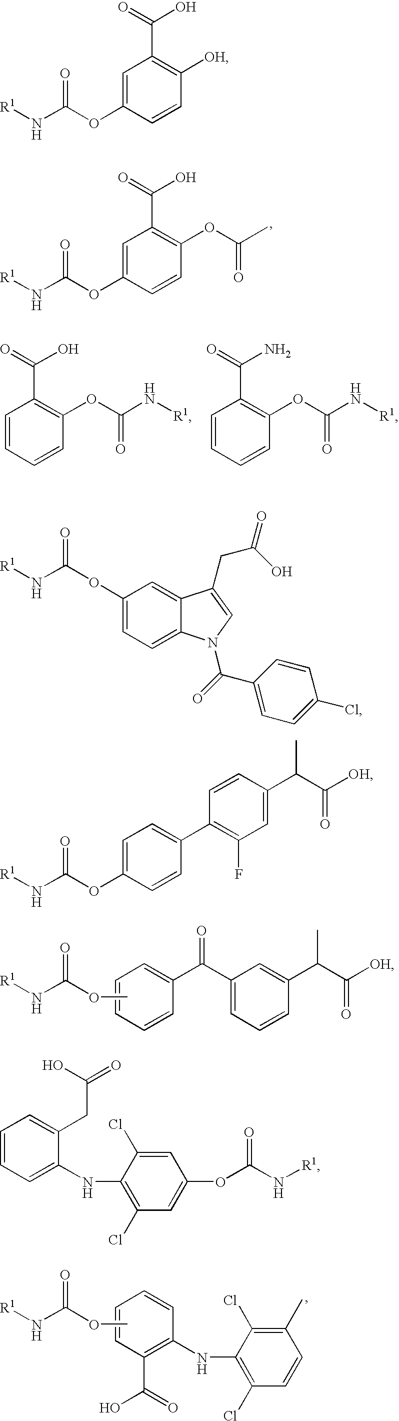

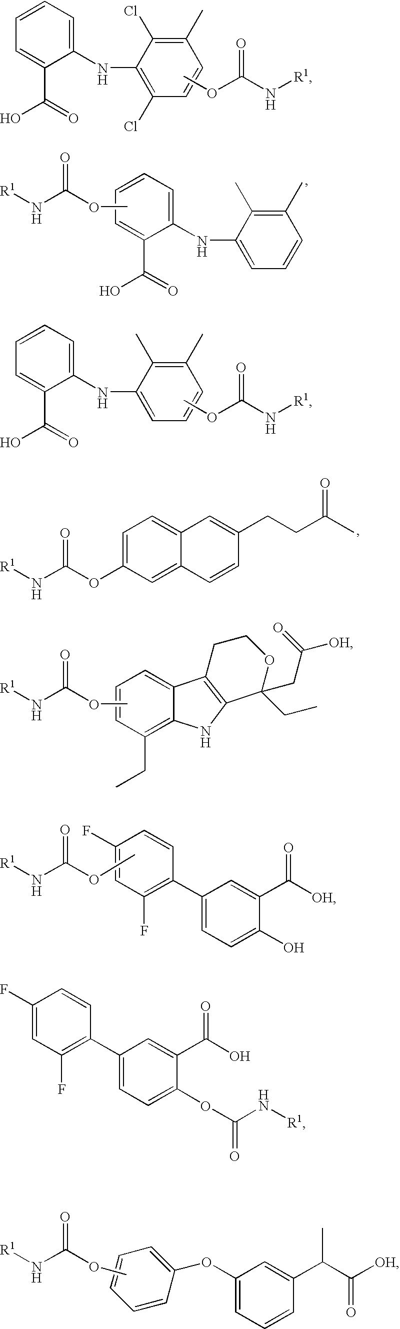

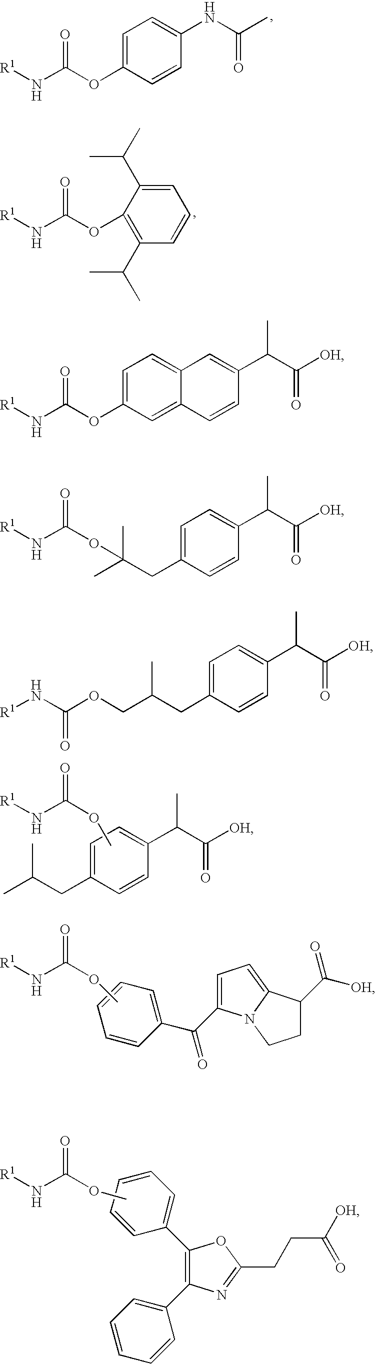

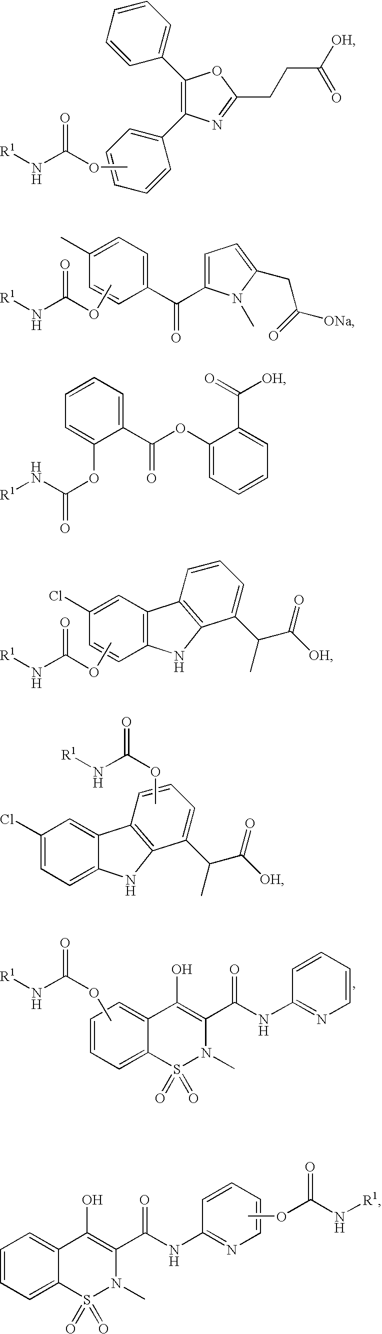

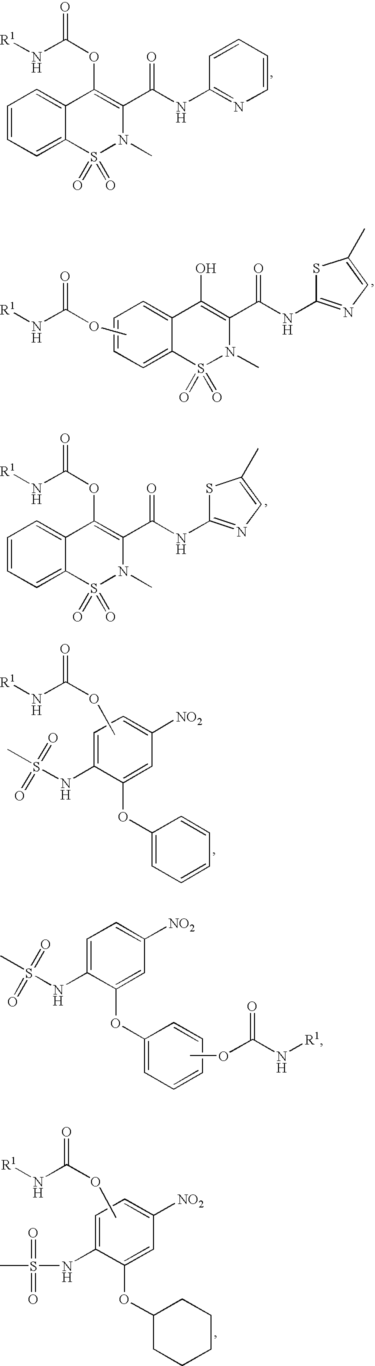

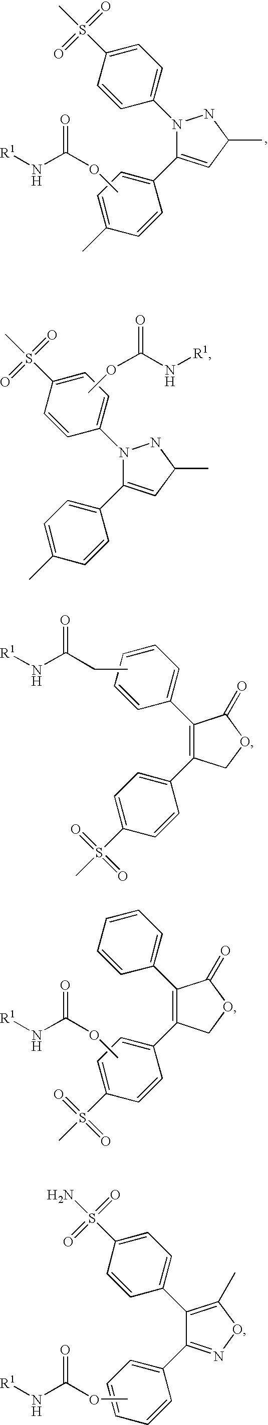

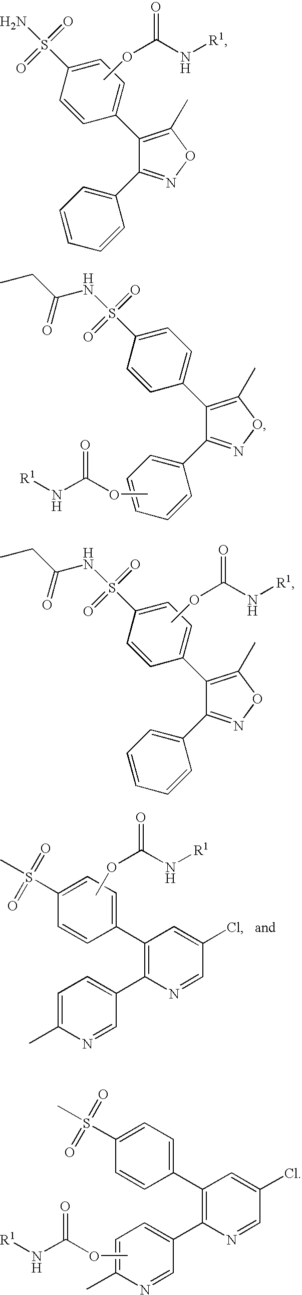

In a further embodiment, the compound of Formula (I) has the structure: 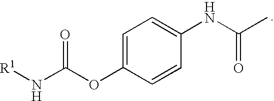
In a further or alternative embodiment, R¹ is selected from cyclohexyl and CH₂cyclohexyl.

In a further embodiment, the compound has the structure:

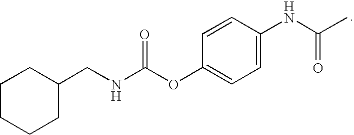

In a further embodiment, the compound has the structure:

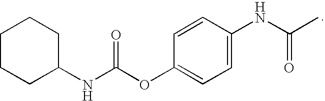

In a further or alternative embodiment, the compound, upon inhibition of fatty acid amide hydrolase (FAAH), produces acetaminophen. In a further embodiment, the inhibition is irreversible inhibition.

In certain embodiments, O-A is the deprotonated form of a hydroxy-containing NSAID selected from among salicylic acid, salicylamide, salsalate, diflunisal, gentisic acid, piroxicam, and meloxicam. In some other embodiments, O-A is a the deprotonated form of a hydroxy-containing NSAID selected from among salicylic acid, salicylamide, salsalate, diflunisal, and gentisic acid.

In other embodiments, O-A is the deprotonated form of a hydroxy-containing NSAID metabolite, wherein the hydroxy-containing NSAID metabolite is a metabolite of an NSAID selected from among acetylsalicylic acid, salicylic acid, salicylamide, salsalate, diflunisal, gentisic acid, indomethacin, sulindac, tolmetin, diclofenac, etodolac, nabumetone, ibuprofen, fenoprofen, ketoprofen, flurbiprofen, suprofen, carprofen, naproxen, ketorolac, oxaprozin, mefenamic acid, meclofenamate sodium, piroxicam, meloxicam, DuP 697, celecoxib, rofecoxib, valdecoxib, nimesulide, ns-398, parecoxib, and etoricoxib.

In some embodiments, O-A is the deprotonated form of a hydroxy-containing NSAID metabolite, wherein the hydroxy-containing NSAID metabolite is a metabolite of an NSAID selected from among indomethacin, sulindac, tolmetin, diclofenac, etodolac, nabumetone, ibuprofen, fenoprofen, ketoprofen, flurbiprofen, suprofen, carprofen, naproxen, ketorolac, oxaprozin, mefenamic acid, meclofenamate sodium, piroxicam, meloxicam, DuP 697, celecoxib, rofecoxib, valdecoxib, nimesulide, ns-398, parecoxib, and etoricoxib.

In other embodiments, O-A is the deprotonated form of the hydroxy-containing NSAID selected from among salicylic acid, salicylamide, salsalate, diflunisal, and gentisic acid.

In some embodiments, O-A is the deprotonated form of a hydroxy-containing NSAID metabolite, wherein the hydroxy-containing NSAID metabolite is a metabolite of an NSAID selected from among indomethacin, nabumetone, ibuprofen, fenoprofen, ketoprofen, flurbiprofen, suprofen, carprofen, and naproxen.

In other embodiments, O-A is the deprotonated form of a hydroxy-containing NSAID metabolite, wherein the hydroxy-containing NSAID metabolite is a metabolite of an NSAID selected from among indomethacin, nabumetone, and naproxen.

In some embodiments, O-A is the deprotonated form of a hydroxy-containing NSAID metabolite, wherein the hydroxy-containing NSAID metabolite is a metabolite of an NSAID selected from among indomethacin, and naproxen. In some embodiments, O-A is the deprotonated form of a hydroxy-containing NSAID metabolite, wherein the hydroxy-containing NSAID metabolite is a metabolite of the biologically more active enantiomer of naproxen. In some embodiments, naproxen is a racemate. In other embodiments, naproxen is a single enantiomer, wherein the single enantiomer of naproxen is the biologically more active enantiomer.

In other embodiments, the compound of Formula (I) has a structure selected from among:

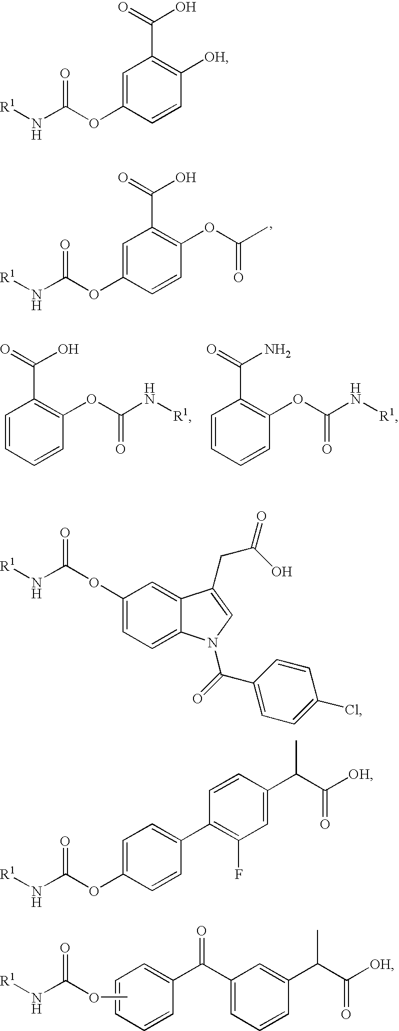

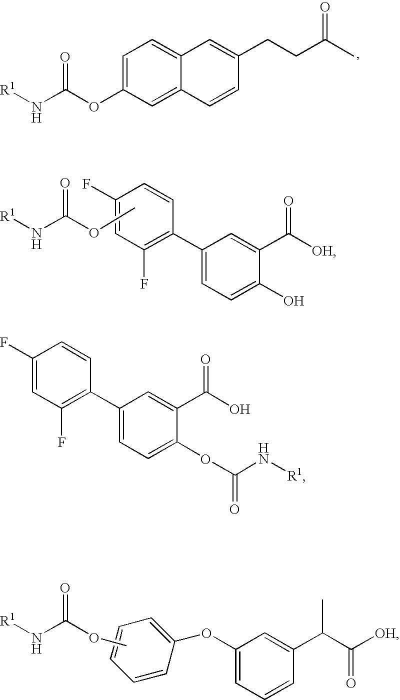

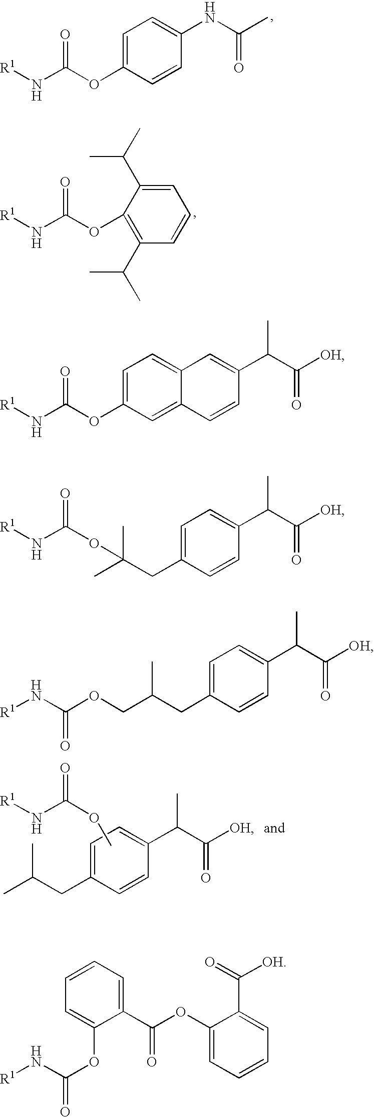

In some embodiments, the compound of Formula (I) has a structure selected from among:

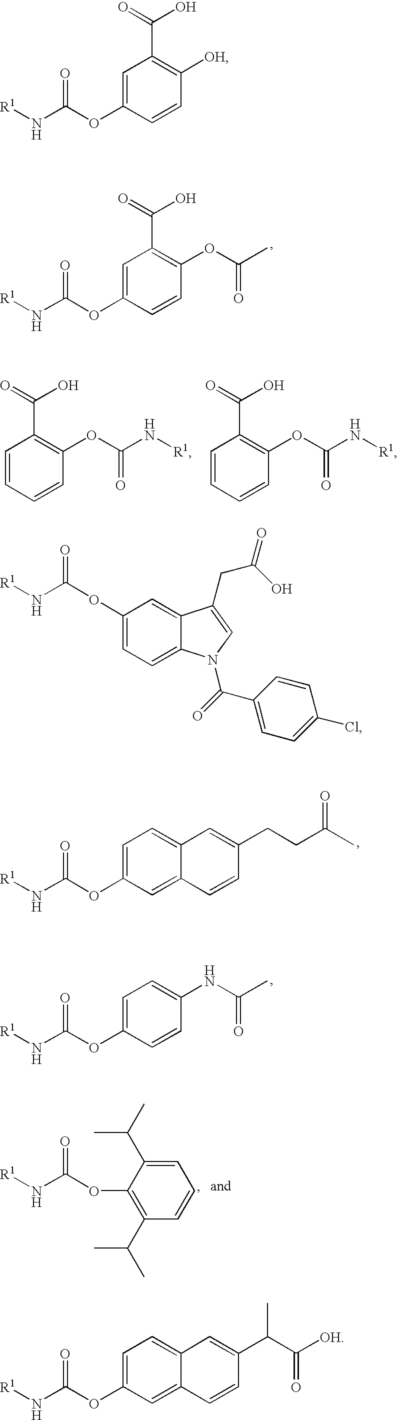

In other embodiments, the compound of Formula (I) has a structure selected from among:

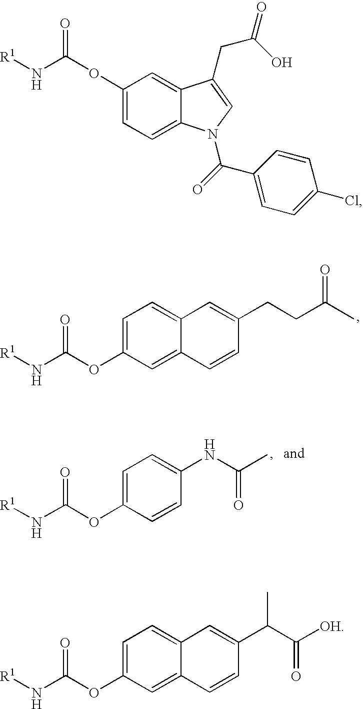

In some embodiments, the compound of Formula (I) has a structure according to:

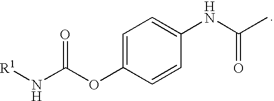

In other embodiments, the compound of Formula (I) has a structure selected from among:

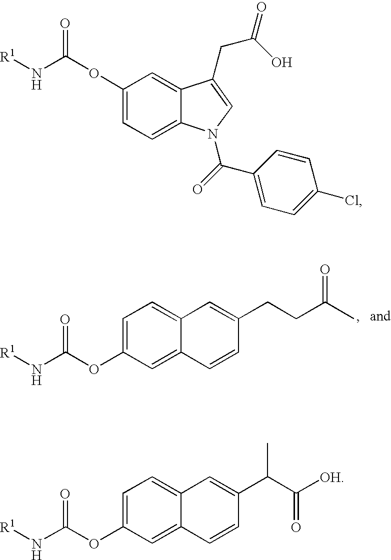

In some other embodiments, the compound of Formula (I) has a structure selected from among:

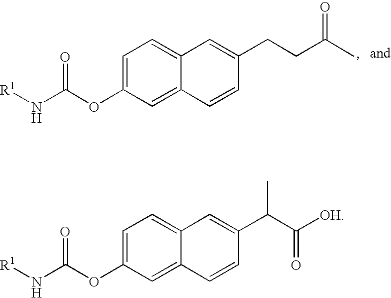

In certain embodiments, R¹ is an optionally substituted group selected from among C₃-C₉ cycloalkyl, and —C₁-C₄alkyl-(C₃-C₉cycloalkyl). In some embodiments, R¹ is an optionally substituted C₃-C₉ cycloalkyl. In certain other embodiments, R¹ is an optionally substituted —C₁-C₄alkyl-(C₃-C₉cycloalkyl). In other embodiments, R¹ is selected from among methyl, ethyl, propyl, isopropyl, n-butyl, sec-butyl, iso-butyl, tert-butyl, pentyl, hexyl, cyclopropyl, cyclobutyl, cyclopentyl, cyclohexyl, cycloheptyl, cyclo-octyl, cyclopropylmethyl, cyclobutylmethyl, cyclopentylmethyl, cyclohexylmethyl, and cycloheptylmethyl. In certain other embodiments, R¹ is selected from among isopropyl, sec-butyl, iso-butyl, tert-butyl, cyclopropyl, cyclobutyl, cyclopentyl, cyclohexyl, cycloheptyl, cyclopropylmethyl, cyclobutylmethyl, cyclopentylmethyl, and cyclohexylmethyl. In some embodiments, R¹ is selected from among isopropyl, sec-butyl, iso-butyl, tert-butyl, cyclopropyl, cyclopentyl, cyclohexyl, cyclopropylmethyl, cyclopentylmethyl, and cyclohexylmethyl. In some other embodiments, R¹ is selected from among isopropyl, sec-butyl, iso-butyl, cyclohexyl, and cyclohexylmethyl. In some embodiments, R¹ is selected from among cyclohexyl and cyclohexylmethyl.

In some embodiments, R¹ is selected from among methyl, ethyl, propyl, isopropyl, n-butyl, sec-butyl, iso-butyl, tert-butyl, pentyl, and hexyl. In other embodiments, R¹ is selected from among propyl, isopropyl, n-butyl, sec-butyl, iso-butyl, tert-butyl, pentyl, and hexyl.

In other embodiments, R¹ is selected from among cyclopropyl, cyclobutyl, cyclopentyl, cyclohexyl, cycloheptyl, and cyclo-octyl. In some other embodiments, R¹ is selected from among cyclopentyl, cyclohexyl, and cycloheptyl. In other embodiments, R¹ is cyclohexyl.

In other embodiments, R¹ is selected from among methyl, ethyl, propyl, isopropyl, n-butyl, sec-butyl, iso-butyl, tert-butyl, pentyl, hexyl, cyclopropyl, cyclobutyl, cyclopentyl, cyclohexyl, cycloheptyl, cyclo-octyl, CH₂cyclopropyl, CH₂cyclobutyl, CH₂cyclopentyl, CH₂cyclohexyl, and CH₂cycloheptyl.

In other embodiments, R¹ is selected from among isopropyl, sec-butyl, iso-butyl, tert-butyl, cyclopropyl, cyclobutyl, cyclopentyl, cyclohexyl, cycloheptyl, CH₂cyclopropyl, CH₂cyclobutyl, CH₂cyclopentyl, CH₂cyclohexyl.

In further embodiments, R¹ is selected from among isopropyl, sec-butyl, iso-butyl, tert-butyl, cyclopropyl, cyclopentyl, cyclohexyl, CH₂cyclopropyl, CH₂cyclopentyl, CH₂cyclohexyl.

In yet further embodiments, R¹ is selected from among isopropyl, sec-butyl, iso-butyl, cyclohexyl, and CH₂cyclohexyl.

In other embodiments, R¹ is selected from among cyclohexyl and CH₂cyclohexyl.

In some embodiments, R¹ is selected from among cyclopropylmethyl, cyclobutylmethyl, cyclopentylmethyl, cyclohexylmethyl, and cycloheptylmethyl. In other embodiments, R¹ is selected from among cyclopentylmethyl, cyclohexylmethyl, and cycloheptylmethyl. In other embodiments, R¹ is cyclohexylmethyl.

Any combination of the groups described above for the various variables is contemplated herein.

In another aspect are processes of preparing an ester of an alkylcarbamic acid comprising:

treating an isocyanate of Formula (II)
O═C═N—R¹ Formula (II)

wherein:

R¹ is an optionally substituted group selected from among C₁-C₆ alkyl, C₃-C₉ cycloalkyl, and —C₁-C₄alkyl-(C₃-C₉cycloalkyl);

with a hydroxy-containing compound selected from acetaminophen, propofol, an analgesic agent, an anti-inflammatory agent, an anti-pyretic agent, an NSAID, a metabolite of an analgesic agent, a metabolite of an anti-inflammatory agent, a metabolite of an anti-pyretic agent, and an NSAID metabolite.

In a further embodiment, the hydroxy-containing compound is acetaminophen. In a further or alternative embodiment, R¹ is selected from among cyclohexyl and CH₂cyclohexyl.

In a further or alternative aspect are esters of the alkylcarbamic acid prepared by the aforementioned processes. In a further embodiment, the ester of an alkylcarbamic acid has the structure 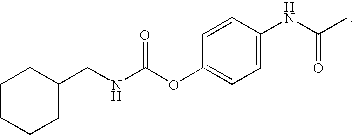
In an alternative embodiment, the ester of an alkylcarbamic acid has the structure

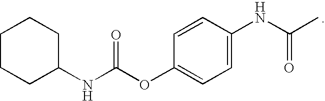

Any combination of the groups described above for the various variables is contemplated herein.

In some embodiments, the hydroxy-containing compound is from an NSAID selected from among salicylic acid, salicylamide, salsalate, diflunisal, gentisic acid, salicylate esters, piroxicam, and meloxicam. In some other embodiments, the hydroxyl moiety is from an NSAID selected from among salicylic acid, salicylamide, salsalate, diflunisal, and gentisic acid.

In some embodiments, the hydroxy-containing compound is from an NSAID metabolite selected from among hydroxy metabolites of acetylsalicylic acid, salicylic acid, salicylamide, salsalate, diflunisal, gentisic acid, salicylate esters, indomethacin, sulindac, tolmetin, diclofenac, etodolac, nabumetone, ibuprofen, fenoprofen, ketoprofen, flurbiprofen, suprofen, carprofen, naproxen, ketorolac, oxaprozin, mefenamic acid, meclofenamate sodium, piroxicam, meloxicam, DuP 697, celecoxib, rofecoxib, valdecoxib, nimesulide, ns-398, parecoxib, and etoricoxib.

In some other embodiments, the hydroxy-containing compound is from an NSAID metabolite selected from among hydroxy metabolites of indomethacin, nabumetone, ibuprofen, fenoprofen, ketoprofen, flurbiprofen, suprofen, carprofen, and naproxen.

In some other embodiments, the hydroxy-containing compound is from an NSAID metabolite selected from among hydroxy metabolites of indomethacin, nabumetone, and naproxen. In some other embodiments, the hydroxy moiety is from an NSAID metabolite selected from among hydroxy metabolites of nabumetone, and naproxen.

In some embodiments, provided herein is an ester of an alkylcarbarmic acid having a structure selected from among:

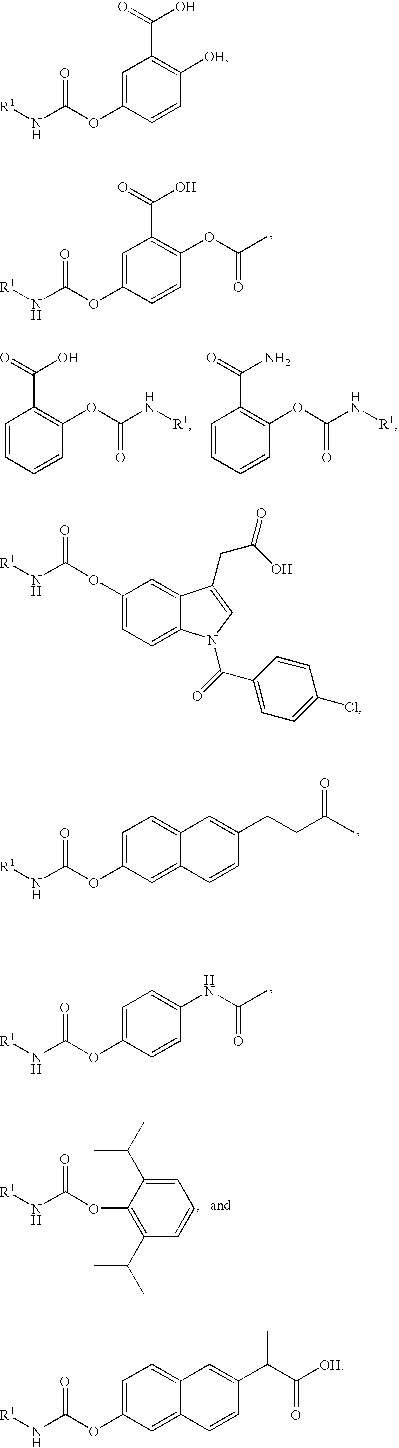

In other embodiments, provided herein is an ester of an alkylcarbamic acid having a structure selected from among:

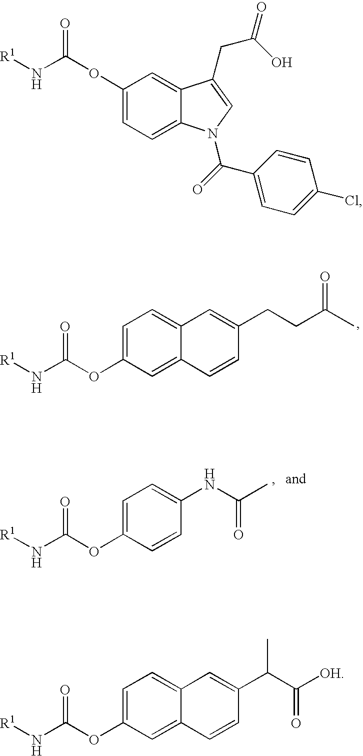

In some other embodiments, provided herein is an ester of an alkylcarbamic acid having the structure according to:

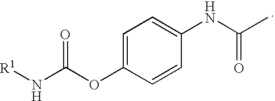

Compounds provided herein, such as, for example, compounds of Formula (I), are esters of alkylcarbamic acids that are formed from acetaminophen, propofol, NSAIDs or NSAID metabolites. In some embodiments, compounds provided herein include a moiety derived from an NSAID or NSAID metabolite. In some embodiments, the NSAID or NSAID metabolite has a chiral center. NSAIDs with a chiral center include, but are not limited, sulindac, etodolac, ibuprofen, fenoprofen, ketoprofen, flurbiprofen, suprofen, carprofen, and naproxen. In some embodiments, compounds provided herein include a moiety derived from a single enantiomer of an NSAID or NSAID metabolite, wherein the single enantiomer of the NSAID or NSAID metabolite is the biologically more active enantiomer. In some embodiments, compounds provided herein, such as, for example, compounds of Formula (I), are single enantiomers. In other embodiments, compounds provided herein are enriched in one enantiomer, preferably the more biologically active enantiomer. In other embodiments, compounds provided herein are racemates. In some embodiments, the biologically more active enantiomer of naproxen is used herein. In some embodiments, the (S)-enantiomer of naproxen is used herein.

Compounds of Formula (I) inhibit FAAH activity through an interaction with FAAH, possibly due to an irreversible (or partially irreversible) nucleophilic attack of an active serine residue (Serine 241) of FAAH on the carbamate moiety of the compounds (Kathuria et al Nature Medicine, vol. 9, no. 1, 76-81, 2003; Deutsch et al Prostaglandins, Leukotrienes and Essential Fatty Acids (2002) 66(2&3), 201-210; Alexander et al Chemistry & Biology, vol. 12, 1179-1187; 2005). Metabolism of the compounds of Formula (I) by the FAAH enzyme results in the hydrolysis of the carbamate compounds and release of the deprotonated form of the hydroxy-containing compound. In some embodiments, are compounds of Formula (I) that bind reversibly to the FAAH enzyme thereby releasing a deprotonated form of a hydroxy containing compound. In some embodiments, the hydroxy containing compound released after reversible binding to the FAAH enzyme is selected from acetaminophen, propofol, an NSAID, and an NSAID metabolite. In other embodiments, are compounds of Formula (I) that partially irreversibly inhibit the FAAH enzyme thereby releasing a hydroxy containing compound. In other embodiments, the hydroxy containing compound released after partial irreversible binding to the FAAH enzyme is selected from acetaminophen, propofol, an NSAID, and an NSAID metabolite. In yet other embodiments, are compounds of Formula (I) that irreversibly inhibit FAAH thereby releasing a deprotonated form of a hydroxy containing compound. In other embodiments, the hydroxy containing compound released after irreversible inhibit the FAAH enzyme is selected from acetaminophen, propofol, an NSAID, and an NSAID metabolite.

Compounds provided herein, such as, for example, compounds of Formula (I), are inhibitors (reversible, partially irreversible and irreversible inhibitors) of fatty acid amide hydrolase (FAAH). Compounds provided herein increase the levels of endogenous fatty acid amides. Compounds provided herein increase the levels of endogenous fatty acid amides selected from among AEA, OEA and PEA. Compounds provided herein, upon inhibition (reversible, partially irreversible and irreversible inhibiton) of fatty acid amide hydrolase (FAAH), produce acetaminophen, propofol, an NSAID, or an NSAID metabolite. In some embodiments, compounds provided herein, upon inhibition (reversible, partially irreversible and irreversible inhibition) of fatty acid amide hydrolase (FAAH), produce an agent that is an analgesic, anti-inflammatory, and/or anti-pyretic, including acetaminophen, an NSAID, or an NSAID metabolite. In other embodiments, compounds provided herein, upon inhibition (reversible, partially irreversible and irreversible inhibition) of fatty acid amide hydrolase (FAAH), produce acetaminophen. In other embodiments, compounds provided herein, upon inhibition (reversible, partially irreversible and irreversible inhibition) of fatty acid amide hydrolase (FAAH), produce an NSAID, or an NSAID metabolite.

In a further aspect are provided pharmaceutical compositions, which include a therapeutically effective amount of at least one of any of the compounds herein, or a pharmaceutically acceptable salt, pharmaceutically acceptable N-oxide, pharmaceutically active metabolite, pharmaceutically acceptable prodrug, or pharmaceutically acceptable solvate. In certain embodiments, the compositions provided herein further include a pharmaceutically acceptable diluent, excipient and/or binder.

Pharmaceutical compositions formulated for administration by an appropriate route and means containing effective concentrations of one or more of the compounds provided herein, or pharmaceutically effective derivatives thereof, that deliver amounts effective for the treatment, prevention, or amelioration of one or more symptoms of diseases, disorders or conditions that are modulated or otherwise affected by FAAH activity, or in which FAAH activity is implicated, are provided. The effective amounts and concentrations are effective for ameliorating any of the symptoms of any of the diseases, disorders or conditions.

Pharmaceutical compositions formulated for administration by an appropriate route and means containing effective concentrations of one or more of the compounds provided herein, or pharmaceutically effective derivatives thereof, that deliver amounts effective for the treatment, prevention, or amelioration of one or more symptoms of pain. In some embodiments the pain is selected from acute or chronic pain, inflammatory diseases, pain, nociceptive pain, neuropathic pain, inflammatory pain, non-inflammatory pain, painful hemorrhagic cystitis, pain associated with the herpes virus, pain associated with diabetes, peripheral neuropathic pain, central pain, deafferentiation pain, chronic nociceptive pain, stimulus of nociceptive receptors, phantom and transient acute pain, peri-operative pain, cancer pain, pain and spasticity associated with multiple sclerosis, central pain, deafferentiation pain, arachnoiditis, radiculopathies, neuralgias, somatic pain, deep somatic pain, surface pain, visceral pain, acute pain, chronic pain, breakthrough pain, chronic back pain, failed back surgery syndrome, fibromyalgia, post-stroke pain, trigeminal neuralgia, sciatica, pain from radiation therapy, complex regional pain syndromes, causalgia, reflex sympathetic dystrophy, phantom limb pain, myofascial pain, and phantom and transient acute pain.

In certain embodiments, provided herein is a pharmaceutical composition containing: i) a physiologically acceptable carrier, diluent, and/or excipient; and ii) one or more compounds provided herein.

In another aspect are methods of treatment comprising administering to a patient having pain, a therapeutically effective amount of a compound, pharmaceutically acceptable salt, pharmaceutically acceptable N-oxide, pharmaceutically active metabolite, pharmaceutically acceptable prodrug, or pharmaceutically acceptable solvate of the compounds of Formula (I) (including any of the subgenera and specific examples provided herein). In one embodiment, is a method of treatment comprising administering to a patient having pain a therapeutically effective amount of the compound, pharmaceutically acceptable salt, pharmaceutically acceptable N-oxide, pharmaceutically active metabolite, pharmaceutically acceptable prodrug, or pharmaceutically acceptable solvate. In a further embodiment, the pain is selected from among acute or chronic pain, inflammatory diseases, pain, nociceptive pain, neuropathic pain, inflammatory pain, non-inflammatory pain, painful hemorrhagic cystitis, pain associated with the herpes virus, pain associated with diabetes, peripheral neuropathic pain, central pain, deafferentiation pain, chronic nociceptive pain, stimulus of nociceptive receptors, phantom and transient acute pain, peri-operative pain, cancer pain, pain and spasticity associated with multiple sclerosis, central pain, deafferentiation pain, arachnoiditis, radiculopathies, neuralgias, somatic pain, deep somatic pain, surface pain, visceral pain, acute pain, chronic pain, breakthrough pain, chronic back pain, failed back surgery syndrome, fibromyalgia, post-stroke pain, trigeminal neuralgia, sciatica, pain from radiation therapy, complex regional pain syndromes, causalgia, reflex sympathetic dystrophy, phantom limb pain, myofascial pain, and phantom and transient acute pain.

In another aspect are uses of the compounds of Formula (I) (including any of the subgenera and specific examples provided herein) for the formulation of a medicament for the treatment of pain. In a further embodiment, the pain is selected from among acute or chronic pain, inflammatory diseases, pain, nociceptive pain, neuropathic pain, inflammatory pain, non-inflammatory pain, painful hemorrhagic cystitis, pain associated with the herpes virus, pain associated with diabetes, peripheral neuropathic pain, central pain, deafferentiation pain, chronic nociceptive pain, stimulus of nociceptive receptors, phantom and transient acute pain, peri-operative pain, cancer pain, pain and spasticity associated with multiple sclerosis, central pain, deafferentiation pain, arachnoiditis, radiculopathies, neuralgias, somatic pain, deep somatic pain, surface pain, visceral pain, acute pain, chronic pain, breakthrough pain, chronic back pain, failed back surgery syndrome, fibromyalgia, post-stroke pain, trigeminal neuralgia, sciatica, pain from radiation therapy, complex regional pain syndromes, causalgia, reflex sympathetic dystrophy, phantom limb pain, myofascial pain, and phantom and transient acute pain.

In another aspect are articles of manufacture, comprising packaging material, the a compound of Formula (I) (including any of the subgenera and specific examples provided herein), which is effective for the treatment of pain, within the packaging material, and a label that indicates that the compound or composition, or pharmaceutically acceptable salt, pharmaceutically acceptable N-oxide, pharmaceutically active metabolite, pharmaceutically acceptable prodrug, or pharmaceutically acceptable solvate thereof, is used for the treatment of pain. In one embodiment, the pain is selected from among acute or chronic pain, inflammatory diseases, pain, nociceptive pain, neuropathic pain, inflammatory pain, non-inflammatory pain, painful hemorrhagic cystitis, pain associated with the herpes virus, pain associated with diabetes, peripheral neuropathic pain, central pain, deafferentiation pain, chronic nociceptive pain, stimulus of nociceptive receptors, phantom and transient acute pain, peri-operative pain, cancer pain, pain and spasticity associated with multiple sclerosis, central pain, deafferentiation pain, arachnoiditis, radiculopathies, neuralgias, somatic pain, deep somatic pain, surface pain, visceral pain, acute pain, chronic pain, breakthrough pain, chronic back pain, failed back surgery syndrome, fibromyalgia, post-stroke pain, trigeminal neuralgia, sciatica, pain from radiation therapy, complex regional pain syndromes, causalgia, reflex sympathetic dystrophy, phantom limb pain, myofascial pain, and phantom and transient acute pain.

In one aspect, provided herein are methods for treating a patient by administering a compound provided herein. In some embodiments, provided herein is a method of inhibiting the activity of fatty acid amide hydrolase or of treating a disease, disorder, or condition, which would benefit from inhibition of fatty acid amide hydrolase activity in a patient, which includes administering to the patient a therapeutically effective amount of at least one of any of the compounds herein, or pharmaceutically acceptable salt, pharmaceutically acceptable N-oxide, pharmaceutically active metabolite, pharmaceutically acceptable prodrug, or pharmaceutically acceptable solvate.

In certain embodiments, compounds and compositions provided herein are effective for the treatment, prevention, or amelioration of one or more symptoms of diseases, disorders or conditions that are selected from among acute or chronic pain, dizziness, vomiting, nausea, eating disorders, neurological and psychiatric pathologies, acute or chronic neurodegenerative diseases, epilepsy, sleep disorders, cardiovascular diseases, renal ischemia, cancers, disorders of the immune system, allergic diseases, parasitic, viral or bacterial infectious diseases, inflammatory diseases, osteoporosis, ocular conditions, pulmonary conditions, gastrointestinal diseases and urinary incontinence.

In some embodiments, compounds and compositions provided herein are effective for the treatment, prevention, or amelioration of one or more symptoms of diseases, disorders or conditions that are selected from among pain, nociceptive pain, neuropathic pain, peri-operative pain, cancer pain, pain and spasticity associated with multiple sclerosis, inflammatory pain, non-inflammatory pain, painful hemorrhagic cystitis, pain associated with the herpes virus, pain associated with diabetes, peripheral neuropathic pain, central pain, deafferentiation pain, chronic nociceptive pain, stimulus of nociceptive receptors, phantom and transient acute pain, depression, anxiety, glaucoma, nausea, emesis, loss of appetite, sleep disturbances, respiratory disorders, allergies, traumatic brain injury, stroke, generalized anxiety disorder (GAD), obsessive compulsive disorders, stress, stress urinary incontinence, attention deficit hyperactivity disorders, schizophrenia, psychosis, Parkinson's disease, muscle spasticity, epilepsy, obesity, hyperlipidemia, insulin resistance syndrome, fatty liver disease, obesity, atherosclerosis, arteriosclerosis, metabolic disorders, feeding and fasting, alteration of appetite, memory, aging, hypertension, septic shock, cardiogenic shock, intestinal inflammation and motility, irritable bowel syndrome, colitis, diarrhea, ileitis, ischemia, cerebral ischemia, hepatic ischemia, myocardial infarction, cerebral excitotoxicity, seizures, febrile seizures, neurotoxicity, neuropathies, sleep, induction of sleep, prolongation of sleep, insomnia, arthritis, rheumatoid arthritis, spondylitis, shoulder tendonitis or bursitis, gouty arthritis, aolymyalgia rheumatica, thyroiditis, hepatitis, inflammatory bowel diseases, asthma, multiple sclerosis, chronic obstructive pulmonary disease (COPD), allergic rhinitis, and cardiovascular diseases.

In some other embodiments, compounds and compositions provided herein are effective for the treatment, prevention, or amelioration of one or more symptoms of diseases, disorders or conditions that are selected from among pain, nociceptive pain, neuropathic pain, peri-operative pain, cancer pain, pain and spasticity associated with multiple sclerosis, inflammatory pain, non-inflammatory pain, painful hemorrhagic cystitis, pain associated with the herpes virus, pain associated with diabetes, peripheral neuropathic pain, central pain, deafferentiation pain, chronic nociceptive pain, stimulus of nociceptive receptors, phantom and transient acute pain, depression, anxiety, glaucoma, nausea, emesis, loss of appetite, sleep disturbances, respiratory disorders, allergies, traumatic brain injury, stroke, generalized anxiety disorder (GAD), obsessive compulsive disorders, stress, stress urinary incontinence, attention deficit hyperactivity disorders, schizophrenia, psychosis, arthritis, rheumatoid arthritis, spondylitis, shoulder tendonitis or bursitis, gouty arthritis, and aolymyalgia rheumatica.

In some other embodiments, compounds and compositions provided herein are effective for the treatment, prevention, or amelioration of one or more symptoms of diseases, disorders or conditions that are selected from among pain, nociceptive pain, neuropathic pain, peri-operative pain, cancer pain, pain and spasticity associated with multiple sclerosis, inflammatory pain, non-inflammatory pain, painful hemorrhagic cystitis, pain associated with the herpes virus, pain associated with diabetes, peripheral neuropathic pain, central pain, deafferentiation pain, chronic nociceptive pain, stimulus of nociceptive receptors, phantom and transient acute pain, arthritis, rheumatoid arthritis, spondylitis, shoulder tendonitis or bursitis, gouty arthritis, and aolymyalgia rheumatica.

In certain embodiments, compounds and compositions provided herein are effective for the treatment, prevention, or amelioration of one or more symptoms of diseases, disorders or conditions that are selected from among pain, nociceptive pain, neuropathic pain, peri-operative pain, cancer pain, pain and spasticity associated with multiple sclerosis, inflammatory pain, non-inflammatory pain, painful hemorrhagic cystitis, pain associated with the herpes virus, pain associated with diabetes, peripheral neuropathic pain, central pain, deafferentiation pain, chronic nociceptive pain, stimulus of nociceptive receptors, phantom and transient acute pain.

In certain other embodiments, compounds and compositions provided herein are effective for the treatment, prevention, or amelioration of one or more symptoms of pain, inflammation and/or fever.

In one aspect, provided herein is a method of inhibiting fatty acid amide hydrolase activity in a mammal, which includes administering to the mammal a therapeutically effective amount of a compound or composition provided herein. In some embodiments the mammal is a human. In other embodiments, the compound or composition is orally administered.

In another aspect, a compound provided herein is used for the formulation of a medicament for the inhibition of fatty acid amide hydrolase (FAAH).

In a further aspect, a compound provided herein is used for the formulation of a medicament for the treatment of pain.

Articles of manufacture containing packaging material, a compound or composition or pharmaceutically acceptable derivative, which is effective for inhibiting the activity of fatty acid amide hydrolase (FAAH), within the packaging material, and a label that indicates that the compound or composition, or pharmaceutically acceptable salt, pharmaceutically acceptable N-oxide, pharmaceutically active metabolite, pharmaceutically acceptable prodrug, or pharmaceutically acceptable solvate thereof, is used for inhibiting the activity of fatty acid amide hydrolase (FAAH), are provided.

In some embodiments, compounds provided herein are used for inhibiting the activity of fatty acid amide hydrolase (FAAH) activity. In some other embodiments, compounds provided herein are used for inhibiting the activity of fatty acid amide hydrolase activity or for the treatment of a disease or condition that would benefit from inhibition of fatty acid amide hydrolase activity.

Compounds provided herein are irreversible inhibitors of fatty acid amide hydrolase. Compounds provided herein, upon irreversible inhibition of fatty acid amide hydrolase activity, release a suitable analgesic, anti-inflammatory, and/or anti-pyretic compound. Irreversible inhibition of FAAH by compounds provided herein, results in hydrolysis of the carbamate compounds and release of an analgesic, anti-inflammatory and/or anti-pyretic agent. Compounds provided herein may also be derivatized into suitable prodrugs. Upon in vivo administration, prodrugs of the esters of alkylcarbamic acids provided herein, such as, for example, prodrugs of compounds of Formula (I), will be metabolized to provide the parent ester of alkylcarbamic acid compound, i.e. compounds of Formula (I) will be formed upon in vivo metabolism of the prodrugs provided herein.

Compounds provided herein increase the levels of endogenous fatty acid amides. In some embodiments, compounds provided herein increase the levels of endogenous fatty acid amides selected from among AEA, OEA and PEA. In some embodiments, compounds provided herein, upon irreversible inhibition of fatty acid amide hydrolase (FAAH), produce acetaminophen, propofol, an NSAID, or an NSAID metabolite. In other embodiments, compounds provided herein, upon irreversible inhibition of fatty acid amide hydrolase (FAAH), produce acetaminophen, an NSAID, or an NSAID metabolite. In some other embodiments, compounds provided herein, upon irreversible inhibition of fatty acid amide hydrolase (FAAH), produce acetaminophen. In yet other embodiments, compounds provided herein, upon irreversible inhibition of fatty acid amide hydrolase (FAAH), produce an NSAID, or an NSAID metabolite.

Other objects, features and advantages of the methods and compositions described herein will become apparent from the following detailed description. It should be understood, however, that the detailed description and the specific examples, while indicating specific embodiments, are given by way of illustration only, since various changes and modifications within the spirit and scope of the present disclosure will become apparent to those skilled in the art from this detailed description. All references cited herein, including patents, patent applications, and publications, are hereby incorporated by reference in their entirety.

### INCORPORATION BY REFERENCE

All publications and patent applications mentioned in this specification are herein incorporated by reference to the same extent as if each individual publication or patent application was specifically and individually indicated to be incorporated by reference.

### DETAILED DESCRIPTION OF THE INVENTION

The novel features are set forth with particularity in the appended claims. A better understanding of the features and advantages of the present disclosure will be obtained by reference to the following detailed description that sets forth illustrative embodiments, in which the principles described herein are utilized.

Disclosed herein are compounds that inhibit the activity the activity of fatty acid amide hydrolase (FAAH), compositions that include the compounds, and methods of their use. Compounds disclosed herein are inhibitors of fatty acid amide hydrolase (FAAH) and are useful in the treatment of diseases, disorders, or conditions that would benefit from the inhibition of fatty acid amide hydrolase and increases in endogenous fatty acid amides. Compounds and compositions provided herein, which include esters of alkylcarbamic acid compounds, are useful in the treatment of diseases, disorders, and/or conditions, which would benefit from inhibition of FAAH in combination with an analgesic, anti-inflammatory, and/or anti-pyretic, including acetaminophen, NSAIDs, or NSAID metabolites, resulting in, for example, reduction in pain, inflammation, and fever, without the toxicities observed with traditional treatments, such as, for example, NSAIDs and acetaminophen taken alone, or in combination. Compounds and compositions are more effective than such conventional treatments in providing relief of pain, inflammation and/or fever and reduce the risks of adverse side effects associated with such traditional treatments.

Acetaminophen

Acetaminophen (N-acetyl-4-aminophenol; paracetamol), belongs to a class of drugs called analgesics (pain relievers) and antipyretics (fever reducers). Acetaminophen acts to relieve pain by elevating the pain threshold and reduces fever through its action on the heat-regulating center of the brain. Antipyretics interfere with those processes by which pyrogenic factors produce fever, but do not appear to lower body temperature in afebrile subjects. It has been historically accepted that the antipyretics exert their actions within the CNS, primarily at the hypothalamic thermoregulatory center but more recent evidence suggests that peripheral actions may also contribute.

Even though acetaminophen has been used clinically for more than a century, its mechanism of action is still not fully understood. It is known that acetaminophen differs from most other non-steroidal anti-inflammatory drugs (NSAIDs), such as aspirin and cyclooxygenase (COX) inhibitors, in that it is a weak anti-inflammatory agent and displays a low incidence of COX-related adverse effects, such as platelet activity and gastrointestinal ulcerogenicity. Thus, while it is known that the analgesic, antipyretic, and anti-inflammatory effects of NSAIDs depend on their ability to inhibit COX-2, the mechanism by which acetaminophen exerts its analgesic and antipyretic effects has not been reconciled. The fact that acetaminophen is an effective antipyretic-analgesic but a weak anti-inflammatory may be due to its greater inhibition of prostaglandin biosynthesis in the CNS than in the periphery.

While acetaminophen has been extensively used to treat pain and to reduce fever, a serious drawback of acetaminophen therapy is its well characterized toxic effects on the liver and kidney, and the potential for liver necrosis as a complication in patients intoxicated with acetaminophen. Overdoses of acetaminophen can produce potentially fatal hepatic necrosis, renal tubular necrosis and hypoglycemic coma.

NSAIDs

Nonsteroidal anti-inflammatory drugs (NSAIDs) are a group of drugs commonly used to treat arthritis because of their analgesic (pain-killing), anti-inflammatory, and antipyretic (fever-reducing) properties. NSAIDs block the activity of the cyclooxygenase (COX) enzymes, via a mechanism distinct from that of acetaminophen, and reduce prostaglandin levels throughout the body. The mechanism of action of NSAIDs is the inhibition of the COX enzymes, which catalyzes the transformation of arachidonic acid to prostaglandins and leukotrienes. Two COX enzymes have been identified, COX-1 and COX-2, and both enzymes produce prostaglandins that promote inflammation, pain, and fever. Prostaglandins are a related family of chemicals that are produced within the cells of the body by the COX enzymes and have several important functions. Prostaglandins promote inflammation, pain, and fever, support the function of platelets that are necessary for the clotting of blood, and protect the lining of the stomach from the damaging effects of acid. However, only COX-1 produces prostaglandins that support platelets and protect the stomach.

By reducing the levels of prostaglandins, which are known to regulate inflammation, pain, and fever (antipyretic), NSAIDs can mediate inflammation, pain, and fever. As a consequence, ongoing inflammation, pain, and fever are reduced. However, treatment with NSAIDs, such as aspirin, often causes adverse gastrointestinal effects such as the formation ulcers in the stomach and an increased risk of bleeding, which is due to inhibition of COX-1, while COX-2 specific inhibitors have been shown to be associated with increased cardiovascular risks. The incidences of the unfavorable side effects increase as the dose of the NSAIDs increase, a problem that limits the therapeutic utility of this class of compounds.

NSAIDs include, but are not limited to, salicylic acids (e.g., aspirin, salicylic acid, gentisic acid, choline magnesium salicylate, choline salicylate, choline magnesium salicylate, choline salicylate, magnesium salicylate, sodium salicylate and diflunisal), proprionic acids (e.g., carprofen, fenoprofen, fenoprofen calcium, flurobiprofen, ibuprofen, ketoprofen, ketolorac, ketorolac tromethamine, naproxen and oxaprozin), acetic acids (e.g. diclofenac, etodolac, indomethacin, sulindac, tolmetin), fenamates (e.g., meclofenamate, meclofenamate sodium, and mefenamic acid), oxicams (piroxicam and meloxicam), COX-2 specific inhibitors (such as, but not limited to, celecoxib, rofecoxib, valdecoxib, parecoxib, etoricoxib, CS-502, JTE-522, L-745,337 and NS398), and others, such as nabutone. A number of chemical classes of such non-steroidal anti-inflammatory drugs have been identified, as described in CRC Handbook of Eicosanoids: Prostaglandins, and Related Lipids, Volume II, Drugs Acting via the Eicosanoids, pages 59-133, CRC Press, Boca Raton, Fla. (1989).

Certain NSAIDs contain a chiral center and are administered as a racemate or enantiomerically-enriched composition. In some cases one enantiomer is biologically more active than the other. Although marketed as a racemate, the (+)-enantiomer of ibuprofen possesses greater activity in vitro than the (−)-enantiomer. The (S)-enantiomer of naproxen is more active than the (R)-isomer.

Metabolites of NSAIDs that are not structurally different from the parent molecule would be expected to have similar pharmacological properties. Most often, NSAIDs are metabolized to hydroxy-containing molecules. In some situations, metabolites of NSAIDs also mediate inflammation, pain, and fever (Radomski et al. Pharmacological Research Communications, 18:1015-1030, 1986; Shen et al, Adv. Drug. Res. 12:90, 1977; Jeremy et al. Prostaglandins Leukot Essent Fatty Acids. 1990 November; 41(3):195-9).

The general anesthetic propofol has been characterized as a competitive inhibitor of FAAH (Patel et al. Br. J. Pharmacol. 2003, 139, 1005-1013). Propofol has been shown to potentiate endogenous GABAergic neurotransmission (Gamma-AminoButyric Acid) and to directly activate the GABA_{A} receptor (Williams et al. J. Neurosci., 22, 7417-7424, 2002). Propofol is a compound that combines enhancement of GABA_{A} function (GABA_{A} agonist) and increased endocannabinoid content and that both of these pharmacological effects contribute to its sedative efficacy. Behavioral effects of GABA_{A} agonists, include, for example, relief of anxiety (anxiolysis), muscle relaxation, sedation, anticonvulsion, and anesthesia.

Thus, many of the existing analgesic, antipyretic, and anti-inflammatory drugs, such as acetaminophen and NSAIDs, are associated with serious side effects. As such, there exists a need for more effective and less toxic drugs which act to relieve pain, reduce fever, and/or prevent inflammation.

The Endocannabinoid System

The brain endocannabinoid signaling system is composed of three elements (Lambert et al. J. Med. Chem. 2005, vol. 48, no. 16, 5059-5087). The first is represented by the G protein-coupled receptors that bind endogenous and exogenous cannabinoid ligands. Two such receptors have been identified, the CB₁ receptor, which is found almost everywhere in the body, but is most abundant in the central nervous system (CNS) (Freund et al Physiol. Rev. 2003; 83:1017-1066); and the CB₂ receptor, which is primarily expressed in immune cells and in hematopoietic cells, but is also present at low levels in the brain (Munro et al. Nature, 1993; 365:61-65; Van Sickle et al. Science 2005; 310:329-332; Hanus et al., Proc. Nat. Acad. Sci., U.S.A., 1999; 96:14228-14233).

The second element is represented by the endocannabinoids, naturally occurring lipid molecules that bind to and activate cannabinoid receptors (Devane et al. Science 1992;258: 1946-1949; Mechoulam et al. Biochem. Pharmacol. 1995;50:83-90; Sugria et al. Biochem. Biophys. Res. Commun. 1995; 215:89-97), are generated on demand by neurons and other cells (Di Marzo et al. Nature 1994;372:686-691; Giuffrida et al. Nat. Nuerosci. 1999; 2:358-363; Stella et al. Nature 2001; 388:773-778), and are rapidly eliminated (Beltramo et al. FEBS Lett. 1997; 403:263-267; Stella et al. Nature 2001; 388:773-778). The third element is represented by the proteins involved in the formation and elimination of the various endocannabinoid ligands (Piomelli, D. Nat Rev. Neurosci. 2003;4:873-884).

Cannabinoid receptors can be activated by endocannabinoids, as well as synthetic ligands. Cannabinoids have been shown to produce analgesia in animal models for both acute and tonic pain, as well as in humans, to control nausea and to lower intraocular pressure (Pertwee, Prog Neurobiol. April 2001; 63(5):569-611.; Walker et al. Chem Phys Lipids. 2002; 121(1-2):159-72). In addition, cannabinoids have been shown to lower body temperature through the activation of cannabinoid CB₁ receptors (Ovadia et al., Stroke. 2003 August; 34(8):2000-6). CB₁ receptors are further believed to have a variety of functions in regulating neurotransmission (GABA, glutamate, and dopamine) with the basal ganglia circuitry areas (Kofalvi et al., J Neurosci. Mar. 16, 2005; 25(11):2874-84).

Anandamide (arachidonoylethanolamide) was the first endocannabinoid substance to be discovered (Devane et al. Science 1992;258:1946-1949; Piomelli, D. Nat Rev. Neurosci. 2003;4:873-884). Current evidence indicates that this lipid-derived mediator is released upon demand by stimulated nuerons (Di Marzo et al. Nature, 1994;372:686-691; Giuffrida et al. Nat. Neurosci. 1999, 2:358-363); activates cannabinoid receptors with high potentcy (Devane et al. Science 1992;258: 1946-1949), and is rapidly eliminated through a two-step process consisting of carrier-mediated internalization followed by intracellular hydrolysis (metabolism) (Beltramo et al. Science 1997; 277:1094-1097; Di Marzo et al. Nature 1994; 372:686-69 1; Hillard et al. J. Lipid Res. 1997; 38:2383-2398).

The endocannabinoids anandamide and 2-arachidonylglycerol (2-AG), both of which produce most of their effects by binding to the CB, receptor, have been shown to be tonically released and can control basal nociceptive thresholds (Meng et al., Nature Sep. 24, 1998; 395(6700):381-3). In particular, anandamide acts as a CB₁ agonist and exhibits pharmacological activity in mice comparable to other synthetic cannabinoids.

Fatty Acid Amide Hydrolase (FAAH)

Fatty acid amide hydrolase (FAAH) is an enzyme that hydrolyzes the fatty acid amide (FAA) family of endogenous signaling lipids. General classes of fatty acid amides include the N-acylethanolamines (NAEs) and fatty acid primary amides (FAPAs). Examples of NAEs include anandamide (AEA), palmitoylethanolamide (PEA) and oleoylethanolamide (OEA). An example of FAPAs includes 9-Z-octadecenamide or oleamide. (McKinney M K, Cravatt B F. 2005. Annu Rev Biochem 74:411-32)]. FAAH can act as a hydrolytic enzyme not only for fatty acid ethanolamides and primary amides, but also for esters, such as, for example, 2-arachidonylglycerol (2-AG), a major endocannabinoid in the brain (Mechoulam et al. Biochem. Pharmacol. 1995; 50:83-90; Stella et al. Nature, 1997; 388:773-778; Suguria et al. Biochem. Biophys. Res. Commun. 1995; 215:89-97)

FAAH is abundantly expressed throughout the CNS, with particularly high levels in the neocortex, hippocampus, and basal ganglia (Freund et al. Physiol. Rev. 2003; 83:1017-1066). FAAH is also detected in the pancreas, brain, kidney, skeletal muscle, placenta, and liver (Giang, D. K. et al. Molecular Characterization of Human and Mouse Fatty Acid Amide Hydrolases. Proc. Natl. Acad. Sci. U.S.A. 1997, 94, 2238-2242.).

Anandamide, or arachidonylethanolamide, is a NAE that acts as an endogenous ligand for the cannabinoid type 1 (CB₁) receptor (Devane W A, et al. 1992. Science 25 8:1946-49). Anandamide is rapidly eliminated through a two-step process consisting of carrier-mediated transport followed by intracellular hydrolysis by FAAH. The hydrolysis of anandamide by FAAH results in the formation of arachidonic acid and ethanolamine. The current postulated catalytic mechanism for hydrolysis of anandamide by FAAH involves nucleophilic attack of amino acid residue Serine 241 of FAAH on the amide moiety of anandamide, resulting in the formation of arachidonic acid and ethanolamine (Deutsch et al. The fatty acid amide hydrolase (FAAH) Prostaglandins, Leukotrienes and Essential Fatty Acids (2002) 66 (2&3), 201-210).

Mutant mice lacking the gene encoding for FAAH display a profound reduction in hydrolysis activity for anandamide and other fatty acid amides and show signs of enhanced anandamide activity at cannabinoid receptors, leading to observable physiological phenomena such as reduced pain sensation (Cravatt B F, et al. 2001. Proc Nat Acad Sci USA 98: 9371-9376). This suggests that therapeutic agents that alter the activity of the FAAH enzyme can increase the actions of anandamide and other fatty acid amides in the body. Such agents may also avoid the multiple, often undesirable effects produced by indiscriminant activation of cannabinoid receptors by administration of Δ9-THC (the active ingredient in marijuana) and other direct-acting cannabinoids.

Many fatty acid amides are known to have analgesic activity. Fatty acid amides, such as, for example, arachidonyl amino acids and anandamide, induce analgesia in animal models of pain (Walker et al. Proc. Natl. Acad. Sci. 96:12198, 1999; Fride et al. Eur. J. Pharmacol. 231:313, 1993).

Many endogenous fatty acid amides, other than anandamide, do not bind the CB₁ receptor. Several of these lipids have been shown to produce specific cellular and behavioral effects, and may represent a large family of endogenous signaling lipids that act in vivo on receptor systems distinct from CB₁. These include palmitoylethanolamide (PEA) (Calignano A, et al. 1998. Nature 394:277-81; Jaggar S I, et al. 1998. Pain 76:189-99; Franklin A, Parmentier-Batteur et al. 2003. J Neurosci 23: 7767-75), stearoylethanolamide (SEA) (Terrazino et al. 2004 FASEB J: 18:1580-82; Maccarrone M, et al. 2002. Biochem J 366:137-44), and oleoylethanolamide (OEA) (deFonseca F R, et al. 2001. Nature 414:209-12; Fu J, et al. 2003. Nature 425:90-93; Fu J, et al. 2005. Neuropharmacology 48(8):1 147-53). Both OEA and PEA have been shown to activate peroxisome proliferator-activated receptor alpha (PPAR-alpha) (Fu J, et al. 2003. Nature 425:90-93; Guzman M, et al. 2004, J Biol Chem 279(27): 27849-54; Lo Verme J, et al. 2005. Cell Mol Life Sci 62(6): 708-16; LoVerme J, et al. 2005. Life Sci 77(14): 1685-98; Lo Verme J, et al. 2005. Mol Pharmacol 67(1): 15-9). Through these actions, OEA and PEA can regulate several biological pathways including, but not limited to, feeding, metabolism, pain and inflammation. Therefore, agents that alter FAAH enzymatic activity can regulate the levels of a variety of fatty acid amides in vivo that, in turn, have therapeutic actions through a variety of targets.

Many fatty acid amides are produced on demand and rapidly degraded by FAAH. As a result, hydrolysis by FAAH is considered to be one of the essential steps in the regulation of fatty acid amide levels in the central nervous system as well as in peripheral tissues and fluids. The broad distribution of FAAH combined with the broad array of biological effects of fatty acid amides (both endocannabinoid and non-endocannabinoid mechanisms) suggests that inhibition of FAAH may lead to altered levels of fatty acid amides in many tissues and fluids and may be useful to treat many different conditions. In some cases, FAAH inhibitors increase the levels of endogenous fatty acid amides. FAAH inhibitors block the degradation of endocannabinoids and increase the tissue levels of these endogenous substances. FAAH inhibitors can be used in this respect in the prevention and treatment of pathologies in which endogenous cannabinoids and or any other substrates metabolized by the FAAH enzyme are involved.

Inhibition of FAAH is expected to lead to an increase in the level of anandamide and other fatty acid amides. This increase in fatty acid amides may lead to an increase in the noiceptive threshold. Thus, in one embodiment, inhibitors of FAAH are useful in the treatment of pain. Such inhibitors might also be useful in the treatment of other disorders that can be treated using fatty acid amides or modulators of cannabinoid receptors, such as, for example, anxiety, eating disorders, cardiovascular disorders, and inflammation.

The various fatty acid ethanolamides have important and diverse physiological functions. As a result, inhibitor molecules that selectively inhibit FAAH enzymatic activity would allow a corresponding selective modulation of the cellular and extra-cellular concentrations of a FAAH substrate. FAAH inhibitors that are biologically compatible could be effective pharmaceutical compounds when formulated as therapeutic agents for any clinical indication where FAAH enzymatic inhibition is desired.

Diseases, disorders, syndromes and/or conditions, that would benefit from inhibition of FAAH enzymatic activity include, for example, Alzheimer's Disease, schizophrenia, depression, alcoholism, addiction, suicide, Parkinson's disease, Huntington's disease, stroke, emesis, miscarriage, embryo implantation, endotoxic shock, liver cirrhosis, atherosclerosis, cancer, traumatic head injury, glaucoma, and bone cement implantation syndrome.

Other diseases, disorders, syndromes and/or conditions that would benefit from inhibition of FAAH activity, include, for example, multiple sclerosis, retinitis, amyotrophic lateral sclerosis, immunodeficiency virus-induced encephalitis, attention-deficit hyperactivity disorder, pain, nociceptive pain, neuropathic pain, inflammatory pain, non-inflammatory pain, painful hemorrhagic cystitis, obesity, hyperlipidemia, metabolic disorders, feeding and fasting, alteration of appetite, stress, memory, aging, hypertension, septic shock, cardiogenic shock, intestinal inflammation and motility, irritable bowel syndrome, colitis, diarrhea, ileitis, ischemia, cerebral ischemia, hepatic ischemia, myocardial infarction, cerebral excitotoxicity, seizures, febrile seizures, neurotoxicity, neuropathies, sleep, induction of sleep, prolongation of sleep, insomnia, and inflammatory diseases.

Neurological and psychological disorders that would benefit from inhibition of FAAH activity include, for example, pain, depression, anxiety, glaucoma, nausea, emesis, loss of appetite, sleep disturbances, respiratory disorders, allergies, traumatic brain injury, stroke, generalized anxiety disorder (GAD), obsessive compulsive disorders, stress, stress urinary incontinence, attention deficit hyperactivity disorders, schizophrenia, psychosis, Parkinson's disease, muscle spasticity, epilepsy, diskenesia, seizure disorders, jet lag, and insomnia.

FAAH inhibitors can also be used in the treatment of a variety of metabolic syndromes, diseases, disorders and/or conditions, including but not limited to, insulin resistance syndrome, diabetes, hyperlipidemia, fatty liver disease, obesity, atherosclerosis and arteriosclerosis.

FAAH inhibitors are useful in the treatment of a variety of painful syndromes, diseases, disorders and/or conditions, including but not limited to those characterized by non-inflammatory pain, inflammatory pain, peripheral neuropathic pain, central pain, deafferentiation pain, chronic nociceptive pain, stimulus of nociceptive receptors, phantom and transient acute pain.

Inhibition of FAAH activity can also be used in the treatment of a variety of conditions involving inflammation. These conditions include, but are not limited to arthritis (such as rheumatoid arthritis, shoulder tendonitis or bursitis, gouty arthritis, and aolymyalgia rheumatica), organ-specific inflammatory diseases (such as thyroiditis, hepatitis, inflammatory bowel diseases), asthma, other autoimmune diseases (such as multiple sclerosis), chronic obstructive pulmonary disease (COPD), allergic rhinitis, and cardiovascular diseases.

In some cases, FAAH inhibitors are useful in preventing neurodegeneration or for neuroprotection.

In addition, it has been shown that when FAAH activity is reduced or absent, one of its substrates, anandamide, acts as a substrate for COX-2, which converts anandamide to prostamides (Weber et al. J. Lipid. Res. 2004; 45:757). Concentrations of certain prostamides may be elevated in the presence of a FAAH inhibitor. Certain prostamides are associated with reduced intraocular pressure and ocular hypotensivity. Thus, in one embodiment, FAAH inhibitors may be useful for treating glaucoma.

NSAIDs have been shown to inhibit FAAH activity in addition to inhibiting COX activity. NSAIDs, such as, for example, ibuprofen, suprofen, ketorolac, fenoprofen, naproxen, ketoprofen, diclofenac (Fowler et al. J. Exp. Pharmacol. Exp. Ther. 283:729-734, 1997), flurbiprofen (Fowler et al. Arch. Biochem. Biophys. 1999, 362, 191-196), and indomethacin (Fowler et al. Br. J. Pharmacol. 2000, 131, 498-504) have been shown to inhibit FAAH activity with potentcies in the low- to high-micromolar range, depending upon the assay used. The data suggests that the use of NSAIDs, such as, for example, ibuprofen, at doses typically administered for conditions such as arthritis may lead to inhibition of FAAH activity (Fowler et al. J Exp. Pharmacol. Exp. Ther. 283:729-734, 1997). However, as noted above, the use of NSAIDs at doses typically administered for conditions such as arthritis lead to undesirable side effects. It has been shown in vitro, that the COX enzymes can metabolize anandamide, and therefore it is reasoned that NSAIDs can also act to prevent COX removal of anandamide and thereby allow its build-up (Guindon et al. Pain 121, 85-93 (2006); Kozak et al., Biochemistry, 30:9041-9049, 2003).

Esters of alkylcarbamic acids and alkylthiocarbamic acids have shown promise as selective FAAH inhibitors (Kathuria et al., Nat. Med. 2003, 9:76-81). A series of alkylcarbamic acid aryl esters, such as, for example, cyclohexylcarbamic acid 3′-carbamoylbiphenyl-3-yl ester (also known as 5′-carbamoylbiphenyl-3-yl cyclohexyl carbamate, UCM597, URB597, and KDS-4103), have been shown to be potent and selective inhibitors of FAAH activity. Alkylcarbamic acid aryl esters, such as, for example, cyclohexylcarbamic acid 3′-carbamoylbiphenyl-3-yl ester, have been shown to be potent and selective inhibitors of FAAH activity, which do not significantly interact with selected serine hydrolases or with cannabinoid receptors (Mor et al. J. Med. Chem. 2004, 47:4998-5008; Piomelli et al. International Patent Publication No. WO 2004/033422; incorporated by reference).

Alkylcarbamic acid aryl esters inhibit FAAH activity through an irreversible interaction with FAAH, possibly due to a nucleophilic attack of an active serine residue (Serine 241) of FAAH on the carbamate moiety of the alkylcarbamic acid aryl ester compounds (Kathuria et al. Nature Medicine, vol. 9, no. 1, 76-81, 2003; Deutsch et al. Prostaglandins, Luekotrienes and Essential Fatty Acids (2002) 66(2&3), 201-210). Metabolism of the alkylcarbamic acid aryl ester inhibitors by the FAAH enzyme results in the hydrolysis of the carbamate compounds and release of the aryloxy portion of the alkylcarbamic acid aryl ester inhibitor.

Provided herein are compound, which are esters of alkylcarbamic acids, compositions that include them, and methods of their use. Compounds provided herein have a structure according to Formula (I): 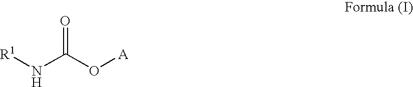
wherein:

X is S or O;

R¹ is an optionally substituted group selected from among C₁-C₆ alkyl, C₃-C₉ cycloalkyl, and —C₁-C₄alkyl-(C₃-C₉cycloalkyl);

O-A is the deprotonated form of a hydroxy-containing compound selected from among acetaminophen, propofol, an NSAID, and an NSAID metabolite; and

pharmaceutically acceptable salts, pharmaceutically acceptable N-oxides, pharmaceutically active metabolites, pharmaceutically acceptable prodrugs, and pharmaceutically acceptable solvates thereof.

Compounds provided herein are irreversible inhibitors of FAAH. In some embodiments, prodrugs of compounds provided herein, such as, for example, prodrugs of compounds of Formula (I), are metabolized in vivo to the parent compound, i.e. compounds of Formula (I). In some embodiments, compounds provided herein are esters of alkylcarbamic acids formed from an isocyanate of Formula (II) (O═C═N—R¹) and a hydroxyl moiety selected from among acetaminophen, propofol, an NSAID, and an NSAID metabolite. In some embodiments, compounds provided herein are esters of alkylthiocarbamic acids formed from an isothiocyanate of Formula (III) (S═C═N—R¹) and a hydroxyl moiety selected from among acetaminophen, propofol, an NSAID, and an NSAID metabolite.

In some embodiments, are compounds of Formula (I) that bind reversibly to the FAAH enzyme thereby releasing a deprotonated form of a hydroxy containing compound. In some embodiments, the hydroxy containing compound released after reversible binding to the FAAH enzyme is selected from acetaminophen, propofol, an NSAID, and an NSAID metabolite. In other embodiments, are compounds of Formula (I) that a partially irreversibly inhibit the FAAH enzyme thereby releasing a hydroxy containing compound. In other embodiments, the hydroxy containing compound released after partial irreversible binding to the FAAH enzyme is selected from acetaminophen, propofol, an NSAID, and an NSAID metabolite. In yet other embodiments, are compounds of Formula (I) that irreversibly inhibit FAAH thereby releasing a deprotonated form of a hydroxy containing compound. In other embodiments, the hydroxy containing compound released after irreversible inhibit the FAAH enzyme is selected from acetaminophen, propofol, an NSAID, and an NSAID metabolite.

In one embodiment, compounds of Formula (I) provided herein are inhibitors of FAAH. In another embodiment, compounds of Formula (I) provided herein are inhibitors of the FAAH enzyme and/or COX enzyme(s). In one embodiment, compounds of Formula (I) provided herein are effective FAAH inhibitors, but are not effective COX inhibitors. In some embodiments, the compounds of Formula (I) provided herein are selectively metabolized by the FAAH enzyme. In certain embodiments, the compounds provided herein are metabolized by the FAAH enzyme resulting in irreversible inhibition of the FAAH enzyme. In some embodiments, compounds provided herein are metabolized by the FAAH enzyme, resulting in the inhibition of FAAH activity and formation of acetaminophen, propofol, an NSAID, or a metabolite of an NSAID. In some embodiments, compounds provided herein are metabolized by the FAAH enzyme, resulting in inhibition of FAAH activity and formation of acetaminophen. In other embodiments, compounds provided herein are metabolized by the FAAH enzyme, resulting in inhibition of FAAH activity and formation of an NSAID or an NSAID metabolite. Thus, the compounds provided herein can exhibit two different phases of activity when administered to a patient. In the initial stage, inhibition of FAAH enzyme activity is observed. In the subsequent or concurrent stage, increasing levels of acetaminophen, propofol, NSAIDs or metabolites of NSAIDs are observed. In some embodiments, compounds provided herein are expected to have reduced renal toxicity compared to conventional treatments that include acetaminophen, propofol, NSAIDs, NSAID metabolites, and/or combinations thereof.

The compounds of Formula (I) provided herein, and pharmaceutical compositions including the compounds, are more effective than traditional therapies (e.g. NSAIDs and acetaminophen taken alone or in combination) in providing relief of pain, fever and inflammation and reduce the risk of adverse side effects associated with such therapies.

Certain Chemical Terminology

Unless defined otherwise, all technical and scientific terms used herein have the same meaning as is commonly understood by one of skill in the art to which the claimed subject matter belongs. All patents, patent applications, published materials referred to throughout the entire disclosure herein, unless noted otherwise, are incorporated by reference in their entirety. In the event that there is a plurality of definitions for terms herein, those in this section prevail. Where reference is made to a URL or other such identifier or address, it is understood that such identifiers can change and particular information on the internet can come and go, but equivalent information can be found by searching the internet. Reference thereto evidences the availability and public dissemination of such information.

It is to be understood that the foregoing general description and the following detailed description are exemplary and explanatory only and are not restrictive of any subject matter claimed. In this application, the use of the singular includes the plural unless specifically stated otherwise. It must be noted that, as used in the specification and the appended claims, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

The section headings used herein are for organizational purposes only and are not to be construed as limiting the subject matter described. All documents, or portions of documents, cited in the application including, but not limited to, patents, patent applications, articles, books, manuals, and treatises are hereby expressly incorporated by reference in their entirety for any purpose.

Definition of standard chemistry terms may be found in reference works, including Carey and Sundberg “ADVANCED ORGANIC CHEMISTRY 4^{TH} ED.” Vols. A (2000) and B (2001), Plenum Press, New York. Unless otherwise indicated, conventional methods of mass spectroscopy, NMR, HPLC, protein chemistry, biochemistry, recombinant DNA techniques and pharmacology, within the skill of the art are employed. Unless specific definitions are provided, the nomenclature employed in connection with, and the laboratory procedures and techniques of, analytical chemistry, synthetic organic chemistry, and medicinal and pharmaceutical chemistry described herein are those known in the art. Standard techniques can be used for chemical syntheses, chemical analyses, pharmaceutical preparation, formulation, and delivery, and treatment of patients. Standard techniques can be used for recombinant DNA, oligonucleotide synthesis, and tissue culture and transformation (e.g., electroporation, lipofection). Reactions and purification techniques can be performed e.g., using kits of manufacturer's specifications or as commonly accomplished in the art or as described herein. The foregoing techniques and procedures can be generally performed of conventional methods well known in the art and as described in various general and more specific references that are cited and discussed throughout the present specification.

An “alkoxy” group refers to a (alkyl)O— group, where alkyl is as defined herein.

An “alkyl” group refers to an aliphatic hydrocarbon group. The alkyl moiety may be a “saturated alkyl” group, which means that it does not contain any alkene or alkyne moieties. The alkyl moiety may also be an “unsaturated alkyl” moiety, which means that it contains at least one alkene or alkyne moiety. An “alkene” moiety refers to a group that has at least one carbon-carbon double bond, and an “alkyne” moiety refers to a group that has at least one carbon-carbon triple bond. The alkyl moiety, whether saturated or unsaturated, may be branched, straight chain, or cyclic. Depending on the structure, an alkyl group can be a monoradical or a diradical (i.e., an alkylene group).

As used herein, C₁-Cₓ includes C₁-C₂, C₁-C₃ . . . C₁-Cₓ.

The “alkyl” moiety may have 1 to 10 carbon atoms (whenever it appears herein, a numerical range such as “1 to 10” refers to each integer in the given range; e.g., “1 to 10 carbon atoms” means that the alkyl group may have 1 carbon atom, 2 carbon atoms, 3 carbon atoms, etc., up to and including 10 carbon atoms, although the present definition also covers the occurrence of the term “alkyl” where no numerical range is designated). The alkyl group of the compounds described herein may be designated as “C₁-C₄ alkyl” or similar designations. By way of example only, “C₁-C₄ alkyl” indicates that there are one to four carbon atoms in the alkyl chain, i.e., the alkyl chain is selected from among methyl, ethyl, propyl, iso-propyl, n-butyl, iso-butyl, sec-butyl, and t-butyl. Thus C₁-C₄ alkyl includes C₁-C₂ alkyl and C₁-C₃ alkyl. Alkyl groups can be substituted or unsubstituted. Typical alkyl groups include, but are in no way limited to, methyl, ethyl, propyl, isopropyl, butyl, isobutyl, tertiary butyl, pentyl, hexyl, ethenyl, propenyl, butenyl, cyclopropyl, cyclobutyl, cyclopentyl, cyclohexyl, and the like.

An “amide” is a chemical moiety with the formula —C(O)NHR or —NHC(O)R, where R is selected from among alkyl, cycloalkyl, aryl, heteroaryl (bonded through a ring carbon) and heteroalicyclic (bonded through a ring carbon). An amide moiety may form a linkage between an amino acid or a peptide molecule and a compound described herein, thereby forming a prodrug. Any amine, or carboxyl side chain on the compounds described herein can be amidified. The procedures and specific groups to make such amides are known to those of skill in the art and can readily be found in reference sources such as Greene and Wuts, Protective Groups in Organic Synthesis, 3^{rd} Ed., John Wiley & Sons, New York, N.Y., 1999, which is incorporated herein by reference in its entirety.

As used herein, the term “aryl” refers to an aromatic ring wherein each of the atoms forming the ring is a carbon atom. Aryl rings can be formed by five, six, seven, eight, nine, or more than nine carbon atoms. Aryl groups can be optionally substituted. Examples of aryl groups include, but are not limited to phenyl, naphthalenyl, phenanthrenyl, anthracenyl, fluorenyl, and indenyl. Depending on the structure, an aryl group can be a monoradical or a diradical (i.e., an arylene group).

An “aryloxy” group refers to an (aryl)O— group, where aryl is as defined herein.

The term “cycloalkyl” refers to a monocyclic or polycyclic radical that contains only carbon and hydrogen, and may be saturated, partially unsaturated, or fully unsaturated. Cycloalkyl groups include groups having from 3 to 10 ring atoms. Illustrative examples of cycloalkyl groups include the following moieties: 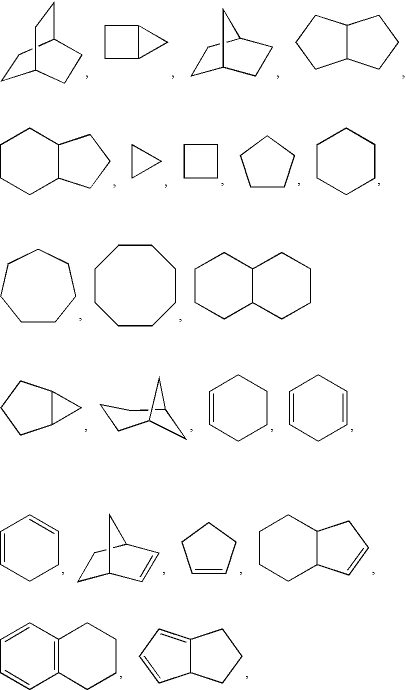
and the like. Depending on the structure, an cycloalkyl group can be a monoradical or a diradical (e.g., an cycloalkylene group).

The term “ester” refers to a chemical moiety with formula —COOR, where R is selected from among alkyl, cycloalkyl, aryl, heteroaryl (bonded through a ring carbon) and heteroalicyclic (bonded through a ring carbon). Any hydroxy, or carboxyl side chain on the compounds described herein can be esterified. The procedures and specific groups to make such esters are known to those of skill in the art and can readily be found in reference sources such as Greene and Wuts, Protective Groups in Organic Synthesis, 3^{rd} Ed., John Wiley & Sons, New York, N.Y., 1999, which is incorporated herein by reference in its entirety.

The term “halo” or, alternatively, “halogen” or “halide” means fluoro, chloro, bromo or iodo.

The terms “heteroaryl” or, alternatively, “heteroaromatic” refers to an aryl group that includes one or more ring heteroatoms selected from nitrogen, oxygen and sulfur. An N-containing “heteroaromatic” or “heteroaryl” moiety refers to an aromatic group in which at least one of the skeletal atoms of the ring is a nitrogen atom. The polycyclic heteroaryl group may be fused or non-fused. Illustrative examples of heteroaryl groups include the following moieties: 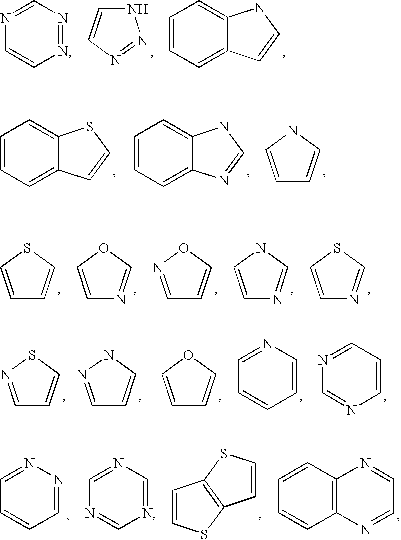
and the like. Depending on the structure, a heteroaryl group can be a monoradical or a diradical (i.e., a heteroarylene group).

An “isocyanato” group refers to a —NCO group.

An “isothiocyanato” group refers to a —NCS group.

The term “moiety” refers to a specific segment or functional group of a molecule. Chemical moieties are often recognized chemical entities embedded in or appended to a molecule.

As used herein, the term “acetyl” refers to a group of formula —C(═O)CH₃.

As used herein, the term “cyano” refers to a group of formula —CN.

As used herein, the substituent “R” appearing by itself and without a number designation refers to a substituent selected from among from alkyl, cycloalkyl, aryl, heteroaryl (bonded through a ring carbon) and non-aromatic heterocycle (bonded through a ring carbon).

The term “optionally substituted” or “substituted” means that the referenced group may be substituted with one or more additional group(s) individually and independently selected from alkyl, cycloalkyl, aryl, heteroaryl, heteroalicyclic, hydroxy, alkoxy, aryloxy, mercapto, alkylthio, arylthio, alkylsulfoxide, arylsulfoxide, alkylsulfone, arylsulfone, cyano, halo, carbonyl, thiocarbonyl, isocyanato, thiocyanato, isothiocyanato, nitro, perhaloalkyl, perfluoroalkyl, silyl, and amino, including mono- and di-substituted amino groups, and the protected derivatives thereof. By way of example an optional substituents may be LₛRₛ, wherein each Lₛ is independently selected from a bond, —O—, —C(═O)—, —S—, —S(═O)—, —S(═O)₂—, —NH—, —NHC(O)—, —C(O)NH—, S(═O)₂NH—, —NHS(═O)₂, —OC(O)NH—, —NHC(O)O—, —(substituted or unsubstituted C₁-C₆ alkyl), or -(substituted or unsubstituted C₂-C₆ alkenyl); and each Rₛ is independently selected from H, (substituted or unsubstituted lower alkyl), (substituted or unsubstituted lower cycloalkyl), heteroaryl, or heteroalkyl. The protecting groups that may form the protective derivatives of the above substituents are known to those of skill in the art and may be found in references such as Greene and Wuts, above.

The compounds presented herein may possess one or more stereocenters and each center may exist in the R or S configuration. The compounds presented herein include all diastereomeric, enantiomeric, and epimeric forms as well as the appropriate mixtures thereof. Stereoisomers may be obtained, if desired, by methods known in the art as, for example, the separation of stereoisomers by chiral chromatographic columns.

The methods and formulations described herein include the use of N-oxides, crystalline forms (also known as polymorphs), or pharmaceutically acceptable salts of compounds described herein, as well as active metabolites of these compounds having the same type of activity. In some situations, compounds may exist as tautomers. All tautomers are included within the scope of the compounds presented herein. In addition, the compounds described herein can exist in unsolvated as well as solvated forms with pharmaceutically acceptable solvents such as water, ethanol, and the like. The solvated forms of the compounds presented herein are also considered to be disclosed herein.

Throughout the specification, groups and substituents thereof can be chosen by one skilled in the field to provide stable moieties and compounds.

Compounds of Formula (I)

Certain compounds that inhibit the activity of fatty acid amide hydrolase (FAAH) play a role in health. In certain embodiments, FAAH inhibitor compounds are useful in treating any of a variety of diseases or conditions. In certain embodiments, compounds provided herein are selective FAAH inhibitor compounds.

Described herein are compounds that inhibit the activity of FAAH. Also described herein are pharmaceutically acceptable salts, pharmaceutically active metabolites and pharmaceutically acceptable prodrugs of such compounds. Pharmaceutical compositions that include at least one such compound or a pharmaceutically acceptable salt, pharmaceutically active metabolite or pharmaceutically acceptable prodrug of such compound, are provided.

Compounds provided herein have a structure according to Formula (I) 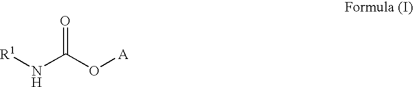
wherein: X is O or S;

R¹ is an optionally substituted group selected from among C₁-C₆ alkyl, C₃-C₉ cycloalkyl, and —C₁-C₄alkyl-(C₃-C₉cycloalkyl);

O-A is the deprotonated form of a hydroxy-containing compound selected from among acetaminophen, propofol, an NSAID, and an NSAID metabolite; and

pharmaceutically acceptable salts, pharmaceutically acceptable N-oxides, pharmaceutically active metabolites, pharmaceutically acceptable prodrugs, and pharmaceutically acceptable solvates thereof.

For any and all of the embodiments, substituents can be selected from among a subset of the listed alternatives. For example, in some embodiments, R¹ is an optionally substituted group selected from among C₃-C₉ cycloalkyl, and —C₁-C₄alkyl-(C₃-C₉cycloalkyl). In other embodiments, R¹ is an optionally substituted C₁-C₆ alkyl. In some embodiments, R¹ is an optionally substituted C₃-C₉ cycloalkyl. In some embodiments, R¹ is an optionally substituted —C₁-C₄alkyl-(C₃-C₉cycloalkyl).

In other embodiments, R¹ is selected from among methyl, ethyl, propyl, isopropyl, n-butyl, sec-butyl, iso-butyl, tert-butyl, pentyl, hexyl, cyclopropyl, cyclobutyl, cyclopentyl, cyclohexyl, cycloheptyl, cyclo-octyl, cyclopropylmethyl, cyclobutylmethyl, cyclopentylmethyl, cyclohexylmethyl, and cycloheptylmethyl. In certain other embodiments, R¹ is selected from among isopropyl, sec-butyl, iso-butyl, tert-butyl, cyclopropyl, cyclobutyl, cyclopentyl, cyclohexyl, cycloheptyl, cyclopropylmethyl, cyclobutylmethyl, cyclopentylmethyl, and cyclohexylmethyl. In some embodiments, R¹ is selected from among isopropyl, sec-butyl, iso-butyl, tert-butyl, cyclopropyl, cyclopentyl, cyclohexyl, cyclopropylmethyl, cyclopentylmethyl, and cyclohexylmethyl. In some other embodiments, R¹ is selected from among isopropyl, sec-butyl, iso-butyl, cyclohexyl, and cyclohexylmethyl. In some embodiments, R¹ is selected from among cyclohexyl and cyclohexylmethyl.

In some embodiments, R¹ is selected from among methyl, ethyl, propyl, isopropyl, n-butyl, sec-butyl, iso-butyl, tert-butyl, pentyl, and hexyl. In other embodiments, R¹ is selected from among propyl, isopropyl, n-butyl, sec-butyl, iso-butyl, tert-butyl, pentyl, and hexyl.

In other embodiments, R¹ is selected from among cyclopropyl, cyclobutyl, cyclopentyl, cyclohexyl, cycloheptyl, and cyclo-octyl. In some other embodiments, R¹ is selected from among cyclopentyl, cyclohexyl, and cycloheptyl. In other embodiments, R¹ is cyclohexyl.

In some embodiments, R¹ is selected from among cyclopropylmethyl, cyclobutylmethyl, cyclopentylmethyl, cyclohexylmethyl, and cycloheptylmethyl. In other embodiments, R¹ is selected from among cyclopentylmethyl, cyclohexylmethyl, and cycloheptylmethyl. In other embodiments, R¹ is cyclohexylmethyl.

In certain embodiments, O-A is the deprotonated form of a hydroxy-containing NSAID selected from among salicylic acid, salicylamide, salsalate, diflunisal, gentisic acid, piroxicam, and meloxicam. In some other embodiments, O-A is a the deprotonated form of a hydroxy-containing NSAID selected from among salicylic acid, salicylamide, salsalate, diflunisal, and gentisic acid.

In other embodiments, O-A is the deprotonated form of a hydroxy-containing NSAID metabolite, wherein the hydroxy-containing NSAID metabolite is a metabolite of an NSAID selected from among acetylsalicylic acid, salicylic acid, salicylamide, salsalate, diflunisal, gentisic acid, indomethacin, sulindac, tolmetin, diclofenac, etodolac, nabumetone, ibuprofen, fenoprofen, ketoprofen, flurbiprofen, suprofen, carprofen, naproxen, ketorolac, oxaprozin, mefenamic acid, meclofenamate sodium, piroxicam, meloxicam, DuP 697, celecoxib, rofecoxib, valdecoxib, nimesulide, ns-398, parecoxib, and etoricoxib. In certain embodiments, O-A is the deprotonated form of a hydroxy-containing NSAID metabolite, wherein the hydroxy-containing NSAID metabolite is a metabolite of a single enantiomer of an NSAID, such as, for example, a hydroxy-containing metabolite of a single enantiomer of naproxen, wherein the single enantiomer is the biologically more active enantiomer. In certain embodiments, O-A is the deprotonated form of a hydroxy-containing NSAID metabolite, wherein the hydroxy-containing NSAID metabolite is a hydroxy-containing metabolite of the (S)-(+)-enantiomer of naproxen.

In some embodiments, O-A is the deprotonated form of a hydroxy-containing NSAID metabolite, wherein the hydroxy-containing NSAID metabolite is a metabolite of an NSAID selected from among indomethacin, sulindac, tolmetin, diclofenac, etodolac, nabumetone, ibuprofen, fenoprofen, ketoprofen, flurbiprofen, suprofen, carprofen, naproxen, ketorolac, oxaprozin, mefenamic acid, meclofenamate sodium, piroxicam, meloxicam, DuP 697, celecoxib, rofecoxib, valdecoxib, nimesulide, ns-398, parecoxib, and etoricoxib.

In some embodiments, O-A is the deprotonated form of a hydroxy-containing NSAID metabolite, wherein the hydroxy-containing NSAID metabolite is a metabolite of an NSAID selected from among indomethacin, nabumetone, ibuprofen, fenoprofen, ketoprofen, flurbiprofen, suprofen, carprofen, and naproxen.

In other embodiments, O-A is the deprotonated form of a hydroxy-containing NSAID metabolite, wherein the hydroxy-containing NSAID metabolite is a metabolite of an NSAID selected from among indomethacin, nabumetone, and naproxen.

In some embodiments, O-A is the deprotonated form of a hydroxy-containing NSAID metabolite, wherein the hydroxy-containing NSAID metabolite is a metabolite of an NSAID selected from among indomethacin, and naproxen.

In some embodiments, O-A is the deprotonated form of acetaminophen. In some other embodiments, O-A is the deprotonated form of propofol.

In some embodiments, the compound of Formula (I) has the structure selected from among:

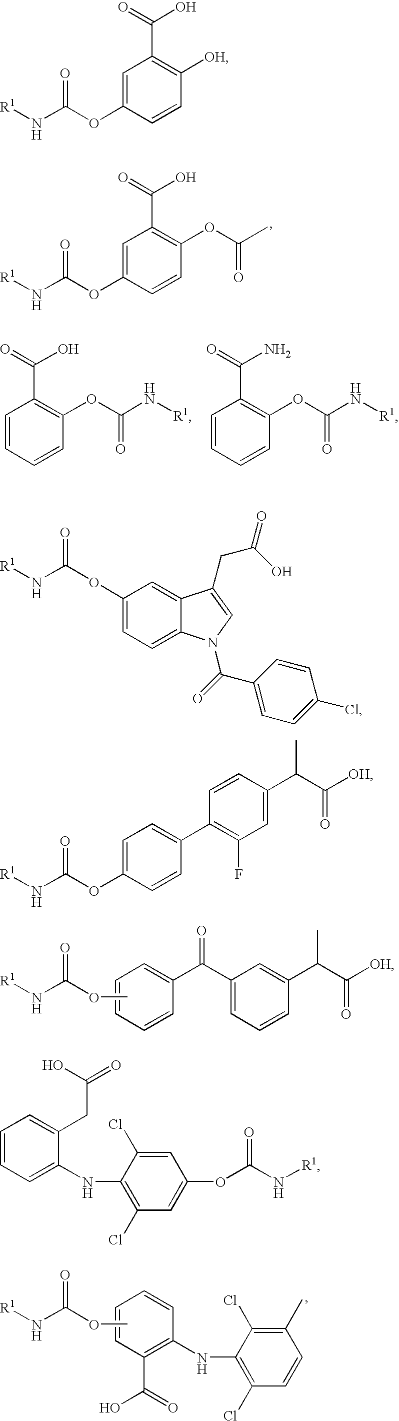

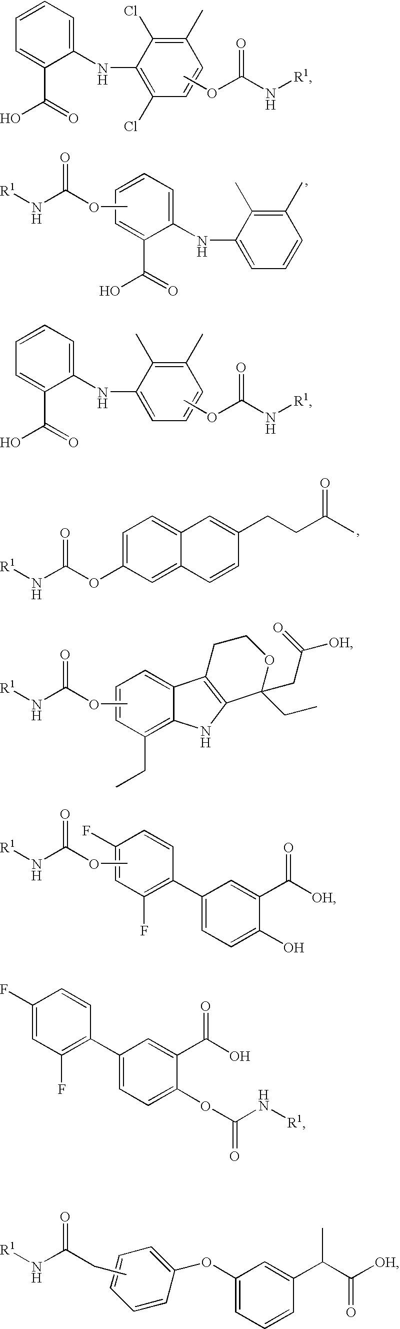

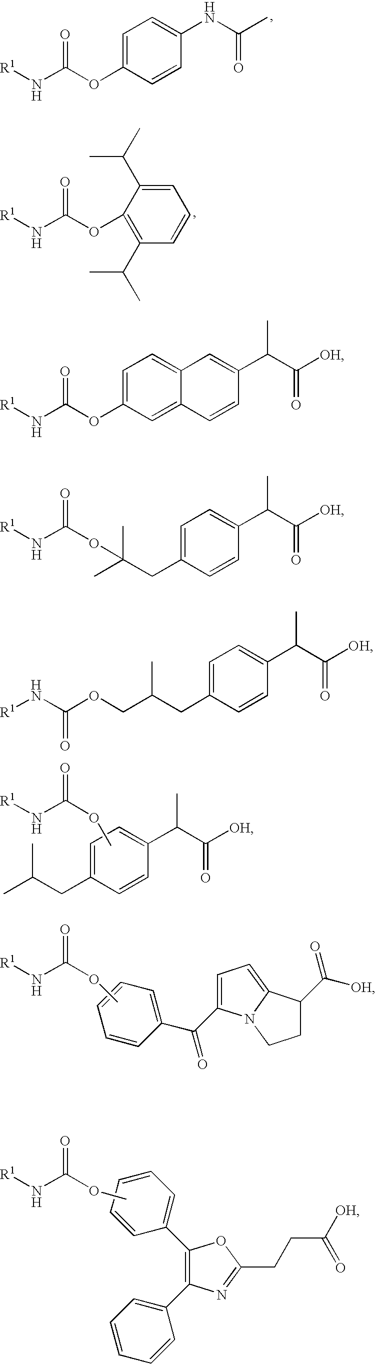

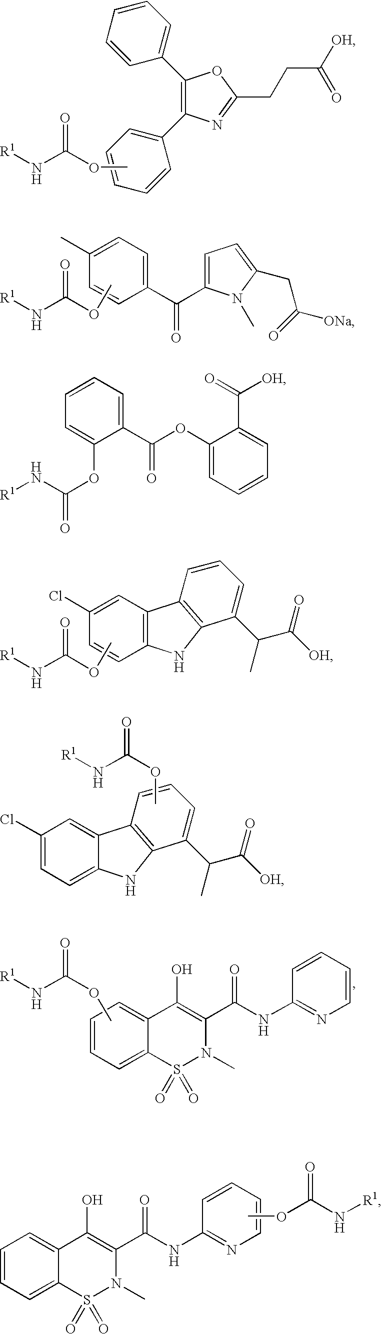

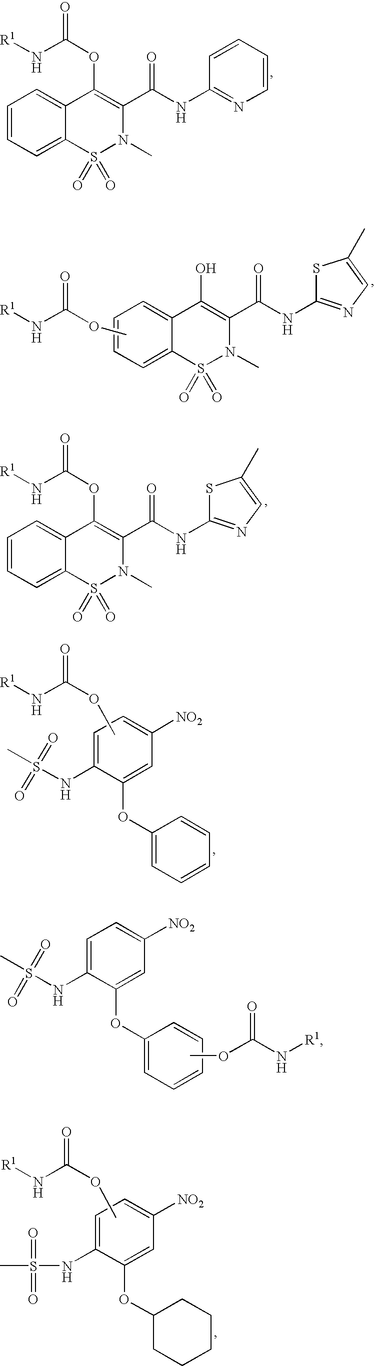

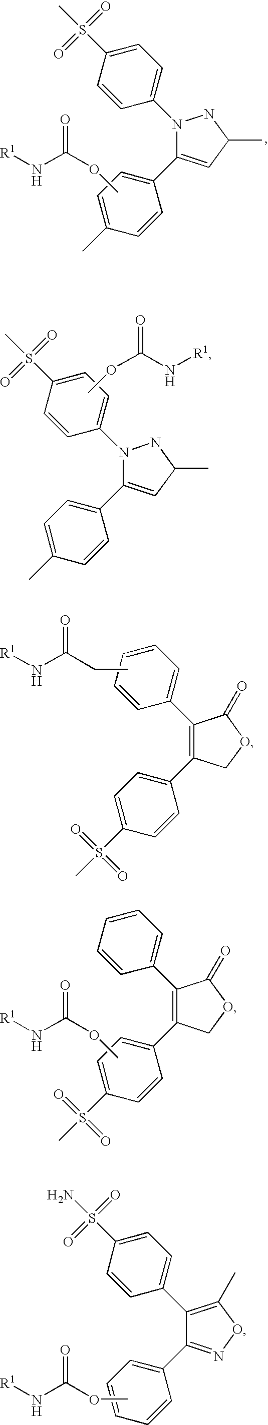

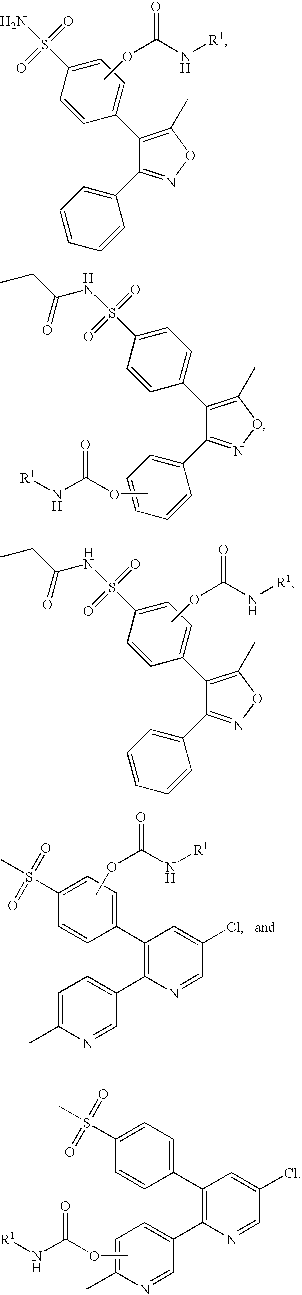

In other embodiments, the compound of Formula (I) has a structure selected from among:

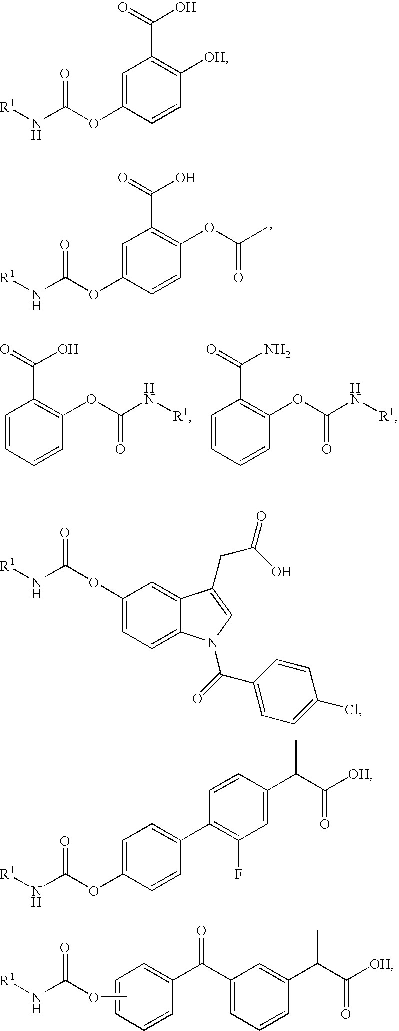

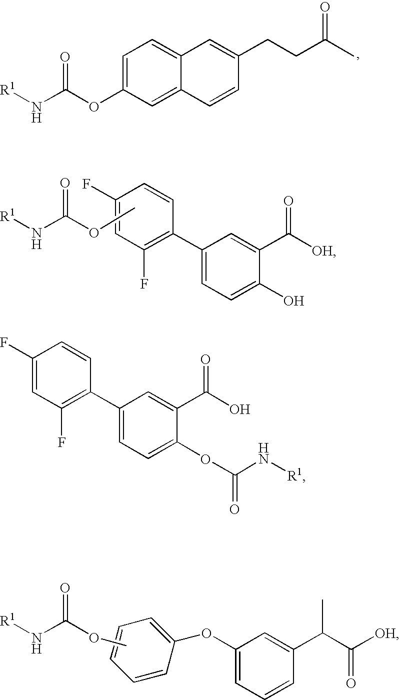

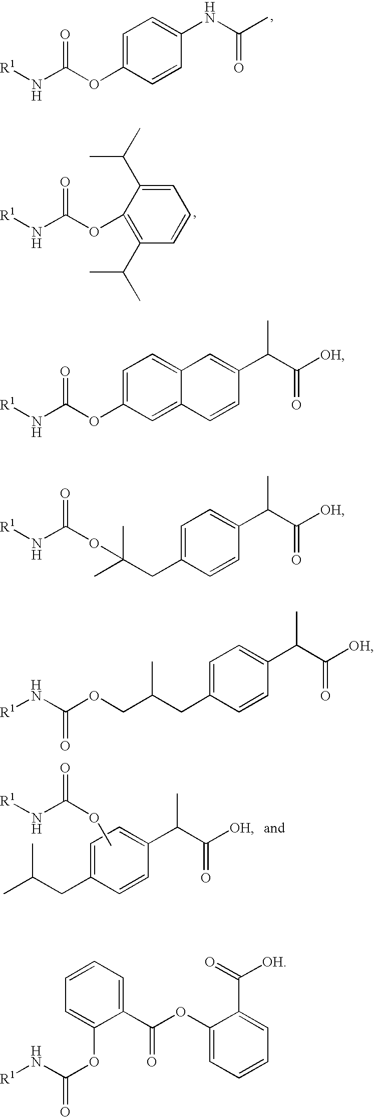

In some embodiments, the compound of Formula (I) has a structure selected from among:

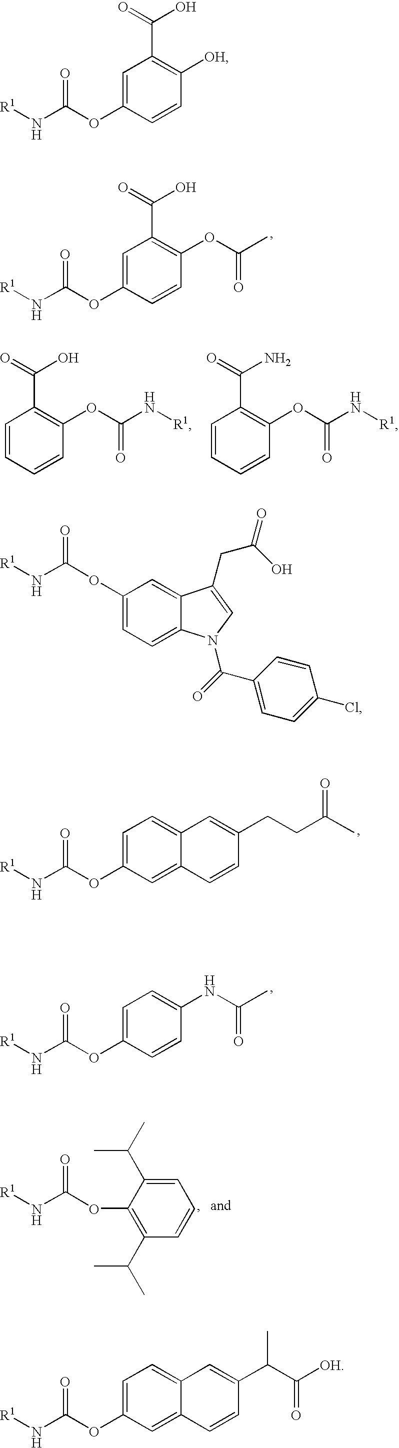

In other embodiments, the compound of Formula (I) has a structure selected from among:

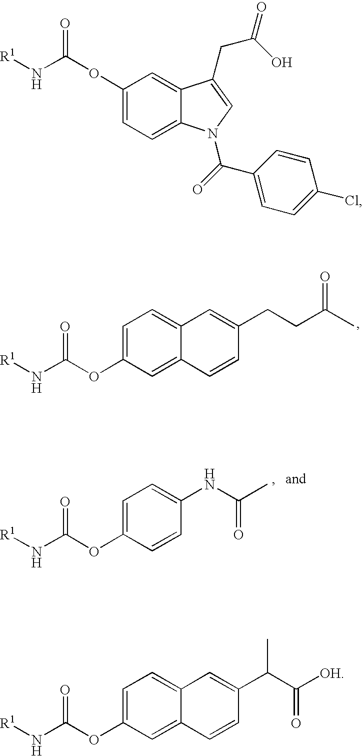

In some embodiments, the compound of Formula (I) has a structure according to:

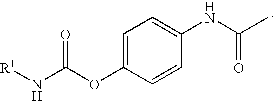

In other embodiments, the compound of Formula (I) has a structure selected from among:

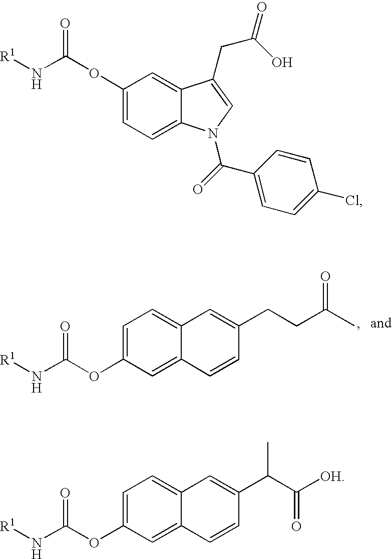

In some other embodiments, the compound of Formula (I) has a structure selected from among:

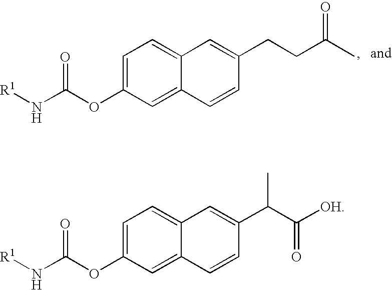

In other embodiments, provided herein is a compound having a structure according to:

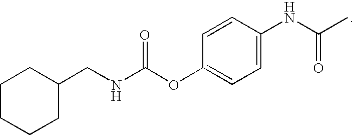

In some embodiments, provided herein is a compound having a structure according to:

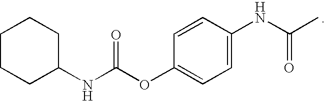

In one aspect is a compound of Formula (IV): 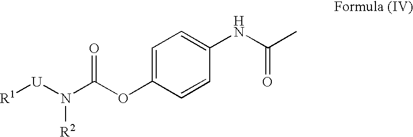
wherein:
U is a bond or CH₂;
R¹ is an optionally substituted group selected from among C₁-C₆ alkyl, C₃-C₈ cycloalkyl, and —C₁-C₃-C₈cycloalkyl);
R² is H or an optionally substituted alkyl; and
pharmaceutically acceptable salts, pharmaceutically acceptable N-oxides, pharmaceutically active metabolites,
pharmaceutically acceptable prodrugs, and pharmaceutically acceptable solvates thereof.

In one embodiment is a compound of Formula (V): 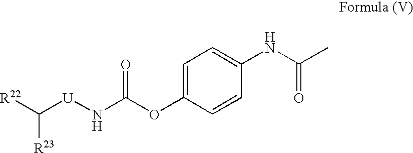
wherein:
U is a bond or CH₂;
R²² and R²³ are individually H, C₁-C₅ alkyl, C₃-C₈ cycloalkyl, and —C₁-C₃alkyl(C₃-C₈cycloalkyl); or
R²² and R²³ together form a 3-, 4-, 5-, 6-, 7-, or 8-membered cycloalkyl; and
pharmaceutically acceptable salts, pharmaceutically acceptable N-oxides, pharmaceutically active metabolites,
pharmaceutically acceptable prodrugs, and pharmaceutically acceptable solvates thereof.

In another embodiment is a compound of Formula (VI): 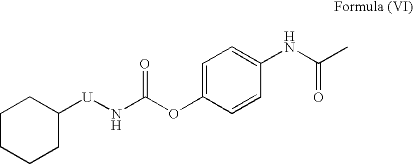
wherein:
U is a bond or CH₂; and
pharmaceutically acceptable salts, pharmaceutically acceptable N-oxides, pharmaceutically active metabolites,
pharmaceutically acceptable prodrugs, and pharmaceutically acceptable solvates thereof.

Any combination of the groups described above for the various variables is contemplated herein.

Preparation of Compounds Described Herein

The synthesis of agents that inhibit the activity of FAAH may be synthesized using standard synthetic techniques known to those of skill in the art or using methods known in the art in combination with methods described herein. As a further guide the following synthetic methods may also be utilized.

Use of Protecting Groups

The term “protecting group” refers to chemical moieties that block some or all reactive moieties and prevent such groups from participating in chemical reactions until the protective group is removed. It is preferred that each protective group be removable by a different means. Protective groups that are cleaved under totally disparate reaction conditions fulfill the requirement of differential removal. Protective groups can be removed by acid, base, and hydrogenolysis. Groups such as trityl, dimethoxytrityl, acetal and t-butyldimethylsilyl are acid labile and may be used to protect carboxy and hydroxy reactive moieties in the presence of amino groups protected with Cbz groups, which are removable by hydrogenolysis, and Fmoc groups, which are base labile. Carboxylic acid and hydroxy reactive moieties may be blocked with base labile groups such as, without limitation, methyl, ethyl, and acetyl in the presence of amines blocked with acid labile groups such as t-butyl carbamate or with carbamates that are both acid and base stable but hydrolytically removable.

Carboxylic acid and hydroxy reactive moieties may also be blocked with hydrolytically removable protective groups such as the benzyl group, while amine groups capable of hydrogen bonding with acids may be blocked with base labile groups such as Fmoc. Carboxylic acid reactive moieties may be protected by conversion to simple ester derivatives as exemplified herein, or they may be blocked with oxidatively-removable protective groups such as 2,4-dimethoxybenzyl, while co-existing amino groups may be blocked with fluoride labile silyl carbamates. In one embodiment, a compound containing both a carboxylic acid reactive moiety and a hydroxy reactive moiety may have one of the reactive moieties blocked while the other reactive moiety is not blocked. In some embodiments, the carboxylic acid reactive moiety may be converted to simple ester derivatives, thus allowing only the hydroxy reactive moiety to participate in subsequent chemical reactions.

Allyl blocking groups are useful in then presence of acid- and base- protecting groups since the former are stable and can be subsequently removed by metal or pi-acid catalysts. For example, an allyl-blocked carboxylic acid can be deprotected with a Pd⁰-catalyzed reaction in the presence of acid labile t-butyl carbamate or base-labile acetate amine protecting groups. Yet another form of protecting group is a resin to which a compound or intermediate may be attached. As long as the residue is attached to the resin, that functional group is blocked and cannot react. Once released from the resin, the functional group is available to react.

Typically blocking/protecting groups may be selected from:

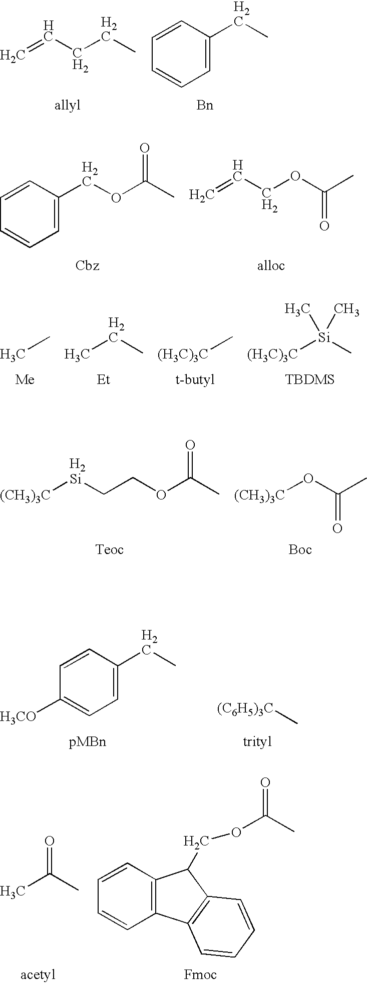

Other protecting groups are described in Greene and Wuts, Protective Groups in Organic Synthesis, 3rd Ed., John Wiley & Sons, New York, N.Y., 1999, which is incorporated herein by reference in its entirety.

Process for the Preparation of Esters of Alkylcarbamic Acids

In certain embodiments, provided herein are methods of making and methods of using FAAH inhibitor compounds provided herein. In certain embodiments, compounds provided herein can be synthesized using the following synthetic schemes. In each scheme, the variables (e.g., A-O, X, and R groups) correspond to the same definitions as those recited above. Compounds may be synthesized using methodologies analogous to those described below by the use of appropriate alternative starting materials.

Described herein are compounds that inhibit the activity of fatty acid amide hydrolase (FAAH) and processes for their preparation. Also described herein are pharmaceutically acceptable salts, pharmaceutically acceptable N-oxides, pharmaceutically active metabolites and pharmaceutically acceptable prodrugs of such compounds. Pharmaceutical compositions that include at least one such compound or a pharmaceutically acceptable salt, pharmaceutically acceptable N-oxide, pharmaceutically active metabolite or pharmaceutically acceptable prodrug of such compound, are provided.

Esters of alkylcarbamic acids disclosed herein are prepared by the general process depicted in Scheme 1. A-OH (2) represents a hydroxy-containing compound selected from among acetaminophen, propofol, an NSAID, and an NSAID metabolite.

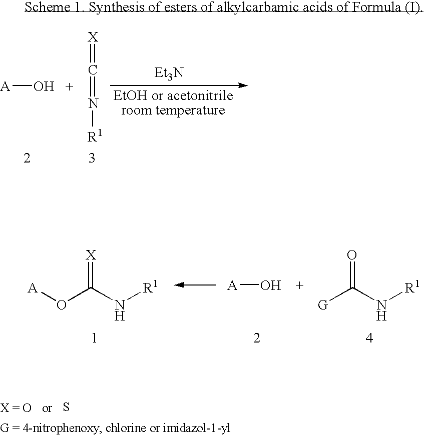

Treatment of A-OH (2) with an isocyanate or isothiocyanate (3) with a base, such as, for example, triethylamine, in an organic solvent, such as, for example, ethanol or acetonitrile, results in the formation of esters of alkylcarbamic acids of structure 1 (see, for example, U.S. Pat. No. 5,112,859; WO 2004/033422; US 2006/0014830; J. Med. Chem. 2004, 47(21); 4998-5008; Tarzia et al. J. Med. Chem. 46:2352-2360 (2003); Kathuria et al. Nature Medicine 9(1): 76 (2003)). Isocyanates or isothiocyanates are commercially available. Methods for the preparation of isocyanates or isothiocyanates (3) are well known in the art. For example, isocyanates (3, X═O) can be prepared from the corresponding carboxylic acid (i.e. R¹—COOH) or acid derivative (e.g. R¹—C(O)Cl) by treatment with an azide source such as, for example, sodium azide or diphenylphosphoryl azide followed by a Curtius-type rearrangement (see, for example, Synth. Commun. 1993, 23, 335; Heterocycles 1993, 36, 1305).

Alternatively, alkylcarbamic acid esters (1) can be prepared by treatment of A-OH (2) with alkylcarbamic acid derivatives of structure (4), where G is 4-nitrophenoxy, chlorine or imidazol-1-yl, in the presence of a base, such as, for example, triethylamine, to provide the desired compound (1). Compounds of structure (4) can be prepared using procedures well known in the art, such as, procedures described in Greene, T. W. and Wuts, P. G. M “Protective Groups in Organic Synthesis”, 3rd Edition, p.549, New York:Wiley, 1999. Breifly, alkylamines (e.g. R¹—NH₂) are treated with phosgene or a phosgene equivalent, such as, for example, trichloromethyl chloroformate or carbonyldiimidazole, to yield compounds of structure (4).

Esters of alkylthiocarbamic acids also can be synthesized by the method outlined in Scheme 2.

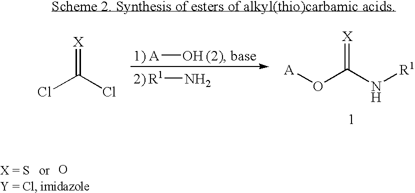

Esters of alkyl(thio)carbamic acids can be prepared by a two-step procedure. Thiophosgene, phosgene, or an equivalent thereof, is first treated with A-OH (2) in the presence of a base in a suitable organic solvent, followed by treatment with an alkylamine, such as, R¹—NH₂. The order of the reaction can be reversed, i.e. thiophosgene, phosgene, or an equivalent thereof, can be treated with the alkylamine followed by A-OH (2). Equivalents of thiophosgene and phosgene include, but are not limited to, 1,1′-thiocarbonyldiimidazole, 1,1′-carbonyldiimidazole, and trichloromethyl chloroformate.

The requisite hydroxy-containing compounds, A-OH (2), can be purchased from commercial sources or prepared using procedures outlined herein.

In one embodiment, acetaminophen is treated with an isocyanate, such as, for example, cyclohexylisocyanate, in the presence of a base, such as, for example, triethylamine in acetonitrile as solvent as depicted in Scheme 3.

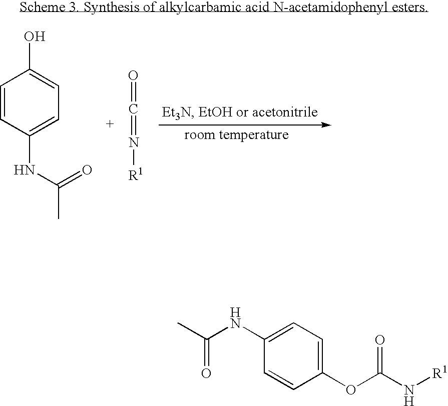

Using the reaction conditions described herein, esters of alkylcarbamic acids, such as structure (1), are obtained in good yields and purity. The compounds prepared by the methods disclosed herein are purified by conventional means known in the art, such as, for example, filtration, recrystallization, chromatography, distillation, and combinations thereof.

In some embodiments, an NSAID that includes a hydroxyl moiety, such as, for example, salicylic acid, may be reacted with isocyanates to form esters of alkylcarbamic acids. In some embodiments, chemical functional groups on the NSAID, other than the hydroxyl moiety, may be protected using a protecting group as discussed above. In some embodiments, a metabolite of an NSAID may be synthesized in order to introduce a hydroxyl group. In some embodiments, a metabolite of an alkoxy containing NSAID, such as, for example, naproxen, indomethacin, nabutone, is prepared by treating the methoxy containing NSAID with a Lewis acid, such as, for example, BBr₃, in order to introduce a hydroxyl group. Scheme 4 depicts the general strategy for deprotecting an alkoxy moiety, such as, for example, a methoxy moiety, in order to furnish a hydroxy-containing metabolite of an NSAID.

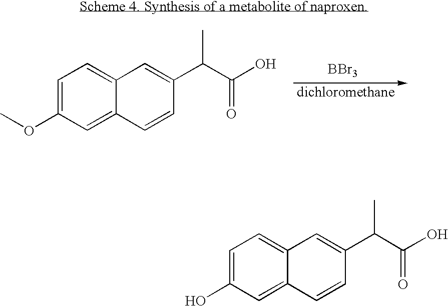

In some embodiments, NSAIDs or NSAID metabolites contain a chiral center and exist as enantiomers, such as, for example, naproxen or metabolites of naproxen. In some embodiments, a single enantiomer of an NSAID or NSAID metabolite is incorporated into the compounds provided herein, such as, for example, compounds of Formula (I). In some embodiments, the biologically more active enantiomer of naproxen is incorporated in the compounds provided herein. In some embodiments, the (S)-enantiomer of naproxen is incorporated in the compounds provided herein.

Any combination of the groups described above for the various variables is contemplated herein.

Pharmaceutical Composition/Formulation

Pharmaceutical compositions may be formulated in a conventional manner using one or more physiologically acceptable carriers including excipients and auxiliaries which facilitate processing of the active compounds into preparations which can be used pharmaceutically. Proper formulation is dependent upon the route of administration chosen. Any of the well-known techniques, carriers, and excipients may be used as suitable and as understood in the art. A summary of pharmaceutical compositions described herein may be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), herein incorporated by reference in their entirety.

Provided herein are pharmaceutical compositions that include a compound described herein, such as, compounds of Formula (I), and a pharmaceutically acceptable diluent(s), excipient(s), or carrier(s). In addition, the compounds described herein can be administered as pharmaceutical compositions in which compounds described herein are mixed with other active ingredients, as in combination therapy. In some embodiments, the pharmaceutical compositions may include other medicinal or pharmaceutical agents, carriers, adjuvants, such as preserving, stabilizing, wetting or emulsifying agents, solution promoters, salts for regulating the osmotic pressure, and/or buffers. In addition, the pharmaceutical compositions can also contain other therapeutically valuable substances.

In certain embodiments, compositions may also include one or more pH adjusting agents or buffering agents, including organic acids such as acetic, citric, lactic, ascorbic, tartaric, maleic, malonic, fumaric, glycolic, succinic, propionic, and methane sulfonic acid; and mineral acids such as phosphoric, hydrobromic, sulfuric, boric, and hydrochloric acids; bases such as sodium hydroxide, sodium phosphate, sodium borate, sodium citrate, sodium acetate, sodium lactate and tris-hydroxymethylaminomethane; and buffers such as citrate/dextrose, sodium bicarbonate and ammonium chloride. Such acids, bases and buffers are included in an amount required to maintain pH of the composition in an acceptable range.

In other embodiments, compositions may also include one or more salts in an amount required to bring osmolality of the composition into an acceptable range. Such salts include those having sodium, potassium or ammonium cations and chloride, citrate, ascorbate, borate, phosphate, bicarbonate, sulfate, thiosulfate or bisulfite anions; suitable salts include sodium chloride, potassium chloride, sodium thiosulfate, sodium bisulfite and ammonium sulfate.

The term “pharmaceutical combination” as used herein, means a product that results from the mixing or combining of more than one active ingredient and includes both fixed and non-fixed combinations of the active ingredients. The term “fixed combination” means that the active ingredients, e.g. a compound described herein and a co-agent, are both administered to a patient simultaneously in the form of a single entity or dosage. The term “non-fixed combination” means that the active ingredients, e.g. a compound described herein and a co-agent, are administered to a patient as separate entities either simultaneously, concurrently or sequentially with no specific intervening time limits, wherein such administration provides effective levels of the two compounds in the body of the patient. The latter also applies to cocktail therapy, e.g. the administration of three or more active ingredients.

A pharmaceutical composition, as used herein, refers to a mixture of a compound described herein, such as, for example, compounds of Formula (I), with other chemical components, such as carriers, stabilizers, diluents, dispersing agents, suspending agents, thickening agents, and/or excipients. The pharmaceutical composition facilitates administration of the compound to an organism. In practicing the methods of treatment or use provided herein, therapeutically effective amounts of compounds described herein are administered in a pharmaceutical composition to a mammal having a disease, disorder, or condition to be treated. Preferably, the mammal is a human. A therapeutically effective amount can vary widely depending on the severity of the disease, the age and relative health of the subject, the potency of the compound used and other factors. The compounds can be used singly or in combination with one or more therapeutic agents as components of mixtures.

The pharmaceutical formulations described herein can be administered to a subject by multiple administration routes, including but not limited to, oral, parenteral (e.g., intravenous, subcutaneous, intramuscular), intranasal, buccal, topical, rectal, or transdermal administration routes. The pharmaceutical formulations described herein include, but are not limited to, aqueous liquid dispersions, self-emulsifying dispersions, solid solutions, liposomal dispersions, aerosols, solid dosage forms, powders, immediate release formulations, controlled release formulations, fast melt formulations, tablets, capsules, pills, delayed release formulations, extended release formulations, pulsatile release formulations, multiparticulate formulations, and mixed immediate and controlled release formulations.

Pharmaceutical compositions including a compound described herein may be manufactured in a conventional manner, such as, by way of example only, by means of conventional mixing, dissolving, granulating, dragee-making, levigating, emulsifying, encapsulating, entrapping or compression processes.

The pharmaceutical compositions will include at least one compound described herein, such as, for example, a compound of Formula (I), as an active ingredient in free-acid or free-base form, or in a pharmaceutically acceptable salt form. In addition, the methods and pharmaceutical compositions described herein include the use of N-oxides, crystalline forms (also known as polymorphs), as well as active metabolites of these compounds having the same type of activity. In some situations, compounds may exist as tautomers. All tautomers are included within the scope of the compounds presented herein. Additionally, the compounds described herein can exist in unsolvated as well as solvated forms with pharmaceutically acceptable solvents such as water, ethanol, and the like. The solvated forms of the compounds presented herein are also considered to be disclosed herein.

Certain Pharmaceutical Terminology

The terms “treat,” “treating” or “treatment,” as used herein, include alleviating, abating or ameliorating a disease or condition symptoms, preventing additional symptoms, ameliorating or preventing the underlying metabolic causes of symptoms, inhibiting the disease or condition, e.g., arresting the development of the disease or condition, relieving the disease or condition, causing regression of the disease or condition, relieving a condition caused by the disease or condition, or stopping the symptoms of the disease or condition either prophylactically and/or therapeutically.

The term “acceptable” with respect to a formulation, composition or ingredient, as used herein, means having no persistent detrimental effect on the general health of the subject being treated.

As used herein, the term “selective binding compound” refers to a compound that selectively binds to any portion of one or more target proteins.

As used herein, the term “selectively binds” refers to the ability of a selective binding compound to bind to a target protein, such as, for example, fatty acid amide hydrolase, with greater affinity than it binds to a non-target protein. In certain embodiments, specific binding refers to binding to a target with an affinity that is at least 10, 50, 100, 250, 500, 1000 or more times greater than the affinity for a non-target.

As used herein, the term “target protein” refers to a molecule or a portion of a protein capable of being bound by a selective binding compound. In certain embodiments, a target protein is fatty acid amide hydrolase (FAAH).

As used herein, amelioration of the symptoms of a particular disease, disorder or condition by administration of a particular compound or pharmaceutical composition refers to any lessening of severity, delay in onset, slowing of progression, or shortening of duration, whether permanent or temporary, lasting or transient that can be attributed to or associated with administration of the compound or composition.

The term “modulate,” as used herein, means to interact with a target either directly or indirectly so as to alter the activity of the target, including, by way of example only, to enhance the activity of the target, to inhibit the activity of the target, to limit the activity of the target, or to extend the activity of the target.

As used herein, the term “modulator” refers to a compound that alters an activity of a molecule. For example, a modulator can cause an increase or decrease in the magnitude of a certain activity of a molecule compared to the magnitude of the activity in the absence of the modulator. In certain embodiments, a modulator is an inhibitor, which decreases the magnitude of one or more activities of a molecule. In certain embodiments, an inhibitor completely prevents one or more activities of a molecule. In certain embodiments, a modulator is an activator, which increases the magnitude of at least one activity of a molecule. In certain embodiments the presence of a modulator results in an activity that does not occur in the absence of the modulator.

As used herein, the term “selective modulator” refers to a compound that selectively modulates a target activity.

As used herein, the term “selective FAAH modulator” refers to a compound that selectively modulates at least one activity associated with FAAH.

As used herein, the term “selectively modulates” refers to the ability of a selective modulator to modulate a target activity to a greater extent than it modulates a non-target activity. In certain embodiments the target activity is selectively modulated by, for example about 2 fold up to more that about 500 fold, in some embodiments, about 2, 5, 10, 50, 100, 150, 200, 250, 300, 350, 400, 450 or more than 500 fold.

As used herein, the term “target activity” refers to a biological activity capable of being modulated by a selective modulator. Certain exemplary target activities include, but are not limited to, binding affinity, signal transduction, enzymatic activity, tumor growth, inflammation or inflammation-related processes, and amelioration of one or more symptoms associated with a disease or condition.

As used herein, the term “agonist” refers to a compound, the presence of which results in a biological activity of a protein that is the same as the biological activity resulting from the presence of a naturally occurring ligand for the protein, such as, for example, fatty acid amide hydrolase (FAAH).

As used herein, the term “partial agonist” refers to a compound the presence of which results in a biological activity of a protein that is of the same type as that resulting from the presence of a naturally occurring ligand for the protein, but of a lower magnitude.

As used herein, the term “antagonist” refers to a compound, the presence of which results in a decrease in the magnitude of a biological activity of a protein. In certain embodiments, the presence of an antagonist results in complete inhibition of a biological activity of a protein, such as, for example, fatty acid amide hydrolase. In certain embodiments, an antagonist is an inhibitor.

As used herein, the IC₅₀ refers to an amount, concentration or dosage of a particular test compound that achieves a 50% inhibition of a maximal response, such as inhibition of FAAH, in an assay that measures such response.

As used herein, EC₅₀ refers to a dosage, concentration or amount of a particular test compound that elicits a dose-dependent response at 50% of maximal expression of a particular response that is induced, provoked or potentiated by the particular test compound.

The term “carrier,” as used herein, refers to relatively nontoxic chemical compounds or agents that facilitate the incorporation of a compound into cells or tissues.

The terms “co-administration” or the like, as used herein, are meant to encompass administration of the selected therapeutic agents to a single patient, and are intended to include treatment regimens in which the agents are administered by the same or different route of administration or at the same or different time.

The terms “effective amount” or “therapeutically effective amount,” as used herein, refer to a sufficient amount of an agent or a compound being administered which will relieve to some extent one or more of the symptoms of the disease or condition being treated. The result can be reduction and/or alleviation of the signs, symptoms, or causes of a disease, or any other desired alteration of a biological system. For example, an “effective amount” for therapeutic uses is the amount of the composition including a compound as disclosed herein required to provide a clinically significant decrease in disease symptoms without undue adverse side effects. An appropriate “effective amount” in any individual case may be determined using techniques, such as a dose escalation study. The term “therapeutically effective amount” includes, for example, a prophylactically effective amount. An “effective amount” of a compound disclosed herein, such as, a compound of Formula (I), is an amount effective to achieve a desired pharmacologic effect or therapeutic improvement without undue adverse side effects. It is understood that “an effect amount” or “a therapeutically effective amount” can vary from subject to subject, due to variation in metabolism of the compound of Formula (I), age, weight, general condition of the subject, the condition being treated, the severity of the condition being treated, and the judgment of the prescribing physician.

The terms “enhance” or “enhancing,” as used herein, means to increase or prolong either in potency or duration a desired effect. Thus, in regard to enhancing the effect of therapeutic agents, the term “enhancing” refers to the ability to increase or prolong, either in potency or duration, the effect of other therapeutic agents on a system. An “enhancing-effective amount,” as used herein, refers to an amount adequate to enhance the effect of another therapeutic agent in a desired system.

The terms “kit” and “article of manufacture” are used as synonyms.

A “metabolite” of a compound disclosed herein is a derivative of that compound that is formed when the compound is metabolized. The term “active metabolite” refers to a biologically active derivative of a compound that is formed when the compound is metabolized. The term “metabolized,” as used herein, refers to the sum of the processes (including, but not limited to, hydrolysis reactions and reactions catalyzed by enzymes, such as, oxidation reactions) by which a particular substance is changed by an organism. Thus, enzymes may produce specific structural alterations to a compound. For example, cytochrome P450 catalyzes a variety of oxidative and reductive reactions while uridine diphosphate glucuronyl transferases catalyze the transfer of an activated glucuronic-acid molecule to aromatic alcohols, aliphatic alcohols, carboxylic acids, amines and free sulfhydryl groups. Further information on metabolism may be obtained from The Pharmacological Basis of Therapeutics, 9th Edition, McGraw-Hill (1996). Metabolites of the compounds disclosed herein can be identified either by administration of compounds to a host and analysis of tissue samples from the host, or by incubation of compounds with hepatic cells in vitro and analysis of the resulting compounds. Both methods are well known in the art. In some embodiments, metabolites of a compound are formed by oxidative processes and correspond to the corresponding hydroxy-containing compound. In some embodiments, a compound is metabolized to pharmacologically active metabolites.

A “prodrug” refers to an agent that is converted into the parent drug in vivo. Prodrugs are often useful because, in some situations, they may be easier to administer than the parent drug. They may, for instance, be bioavailable by oral administration whereas the parent is not. The prodrug may also have improved solubility in pharmaceutical compositions over the parent drug. An example, without limitation, of a prodrug would be a compound described herein, which is administered as an ester (the “prodrug”) to facilitate transmittal across a cell membrane where water solubility is detrimental to mobility but which then is metabolically hydrolyzed to the carboxylic acid, the active entity, once inside the cell where water-solubility is beneficial. A further example of a prodrug might be a short peptide (polyaminoacid) bonded to an acid group where the peptide is metabolized to reveal the active moiety. In certain embodiments, upon in vivo administration, a prodrug is chemically converted to the biologically, pharmaceutically or therapeutically more active form of the compound. In certain embodiments, a prodrug is enzymatically metabolized by one or more steps or processes to the biologically, pharmaceutically or therapeutically active form of the compound. To produce a prodrug, a pharmaceutically active compound is modified such that the active compound will be regenerated upon in vivo administration. The prodrug can be designed to alter the metabolic stability or the transport characteristics of a drug, to mask side effects or toxicity, to improve the flavor of a drug or to alter other characteristics or properties of a drug. By virtue of knowledge of pharmacodynamic processes and drug metabolism in vivo, those of skill in this art, once a pharmaceutically active compound is known, can design prodrugs of the compound. (see, for example, Nogrady ( 1985) Medicinal Chemistry A Biochemical Approach, Oxford University Press, New York, pages 388-392; Silverman (1992), The Organic Chemistry of Drug Design and Drug Action, Academic Press, Inc., San Diego, pages 352-401).

By “pharmaceutically acceptable,” as used herein, refers to a material, such as a carrier or diluent, which does not abrogate the biological activity or properties of the compound, and is relatively nontoxic, i.e., the material may be administered to an individual without causing undesirable biological effects or interacting in a deleterious manner with any of the components of the composition in which it is contained.

The term “pharmaceutically acceptable salt” refers to a formulation of a compound that does not cause significant irritation to an organism to which it is administered and does not abrogate the biological activity and properties of the compound. Pharmaceutically acceptable salts may be obtained by reacting a compound described herein, with acids such as hydrochloric acid, hydrobromic acid, sulfuric acid, nitric acid, phosphoric acid, methanesulfonic acid, ethanesulfonic acid, p-toluenesulfonic acid, salicylic acid and the like. Pharmaceutically acceptable salts also may be obtained by reacting a compound described herein with a base to form a salt such as an ammonium salt, an alkali metal salt, such as a sodium or a potassium salt, an alkaline earth metal salt, such as a calcium or a magnesium salt, a salt of organic bases such as dicyclohexylamine, N-methyl-D-glucamine, tris(hydroxymethyl)methylamine, and salts with amino acids such as arginine, lysine, and the like, or by other methods known in the art.

“Bioavailability” refers to the percentage of the weight of compounds disclosed herein, such as, compounds of Formula (I), dosed that is delivered into the general circulation of the animal or human being studied. The total exposure (AUC_{(0-∞)}) of a drug when administered intravenously is usually defined as 100% bioavailable (F%). “Oral bioavailability” refers to the extent to which compounds disclosed herein, such as, compounds of Formula (I), are absorbed into the general circulation when the pharmaceutical composition is taken orally as compared to intravenous injection.

“Blood plasma concentration” refers to the concentration of compounds disclosed herein, such as, compounds of Formula (I), in the plasma component of blood of a subject. It is understood that the plasma concentration of compounds of Formula (I) may vary significantly between subjects, due to variability with respect to metabolism and/or possible interactions with other therapeutic agents. In accordance with one embodiment disclosed herein, the blood plasma concentration of the compounds of Formula (I) may vary from subject to subject. Likewise, values such as maximum plasma concentration (Cₘₐₓ) or time to reach maximum plasma concentration (Tₘₐₓ), or total area under the plasma concentration time curve (AUC_{(0-∞)}) may vary from subject to subject. Due to this variability, the amount necessary to constitute “a therapeutically effective amount” of a compound of Formula (I) may vary from subject to subject.

“Pharmacodynamics” refers to the factors which determine the biologic response observed relative to the concentration of drug at a site of action.

“Pharmacokinetics” refers to the factors which determine the attainment and maintenance of the appropriate concentration of drug at a site of action.

“Steady state,” as used herein, is when the amount of drug administered is equal to the amount of drug eliminated within one dosing interval resulting in a plateau or constant plasma drug exposure.

Dosage Forms

The compositions described herein can be formulated for administration to a subject via any conventional means including, but not limited to, oral, parenteral (e.g., intravenous, subcutaneous, intrathecal, or intramuscular), buccal, intranasal, epidural, pulmonary, local, rectal or transdermal administration routes. As used herein, the term “subject” is used to mean an animal, preferably a mammal, including a human or non-human. The terms patient and subject may be used interchangeably.

Conventional pharmacological techniques include, e.g., one or a combination of methods: (1) dry mixing, (2) direct compression, (3) milling, (4) dry or non-aqueous granulation, (5) wet granulation, or (6) fusion. See, e.g., Lachman et al., The Theory and Practice of Industrial Pharmacy (1986). Other methods include, e.g., spray drying, pan coating, melt granulation, granulation, fluidized bed spray drying or coating (e.g., wurster coating), tangential coating, top spraying, tableting, extruding and the like.

The pharmaceutical solid dosage forms described herein can include a compound of Formula (I), and one or more pharmaceutically acceptable additives such as a compatible carrier, binder, filling agent, suspending agent, flavoring agent, sweetening agent, disintegrating agent, dispersing agent, surfactant, lubricant, colorant, diluent, solubilizer, moistening agent, plasticizer, stabilizer, penetration enhancer, wetting agent, anti-foaming agent, antioxidant, preservative, or one or more combination thereof, as described in the standard reference Gennaro, A. R. et al., Remington: The Science and Practice of Pharmacy (20th Edition, Lippincott Williams & Wilkins, 2000, see especially Part 5: Pharmaceutical Manufacturing).

Liquid formulation dosage forms for oral administration can be aqueous suspensions selected from the group including, but not limited to, pharmaceutically acceptable aqueous oral dispersions, emulsions, solutions, elixirs, gels, and syrups. See, e.g., Singh et al., Encyclopedia of Pharmaceutical Technology, 2^{nd} Ed., pp. 754-757 (2002). In addition to the particles of compound of Formula (I), the liquid dosage forms may include additives, such as: (a) disintegrating agents; (b) dispersing agents; (c) wetting agents; (d) at least one preservative, (e) viscosity enhancing agents, (f) at least one sweetening agent, and (g) at least one flavoring agent. In some embodiments, the aqueous dispersions can further include a crystalline inhibitor.

Methods of Dosing and Treatment Regimens

The compounds described herein can be used in the preparation of medicaments for the inhibition of fatty acid amide hydrolase, or for the treatment of diseases or conditions that would benefit, at least in part, from inhibition of fatty acid amide hydrolase. In addition, a method for treating any of the diseases or conditions described herein in a subject in need of such treatment, involves administration of pharmaceutical compositions containing at least one compound of Formula (I) described herein, or a pharmaceutically acceptable salt, pharmaceutically acceptable N-oxide, pharmaceutically active metabolite, pharmaceutically acceptable prodrug, or pharmaceutically acceptable solvate thereof, in therapeutically effective amounts to the subject.

The compositions containing the compound(s) described herein can be administered for prophylactic and/or therapeutic treatments. In therapeutic applications, the compositions are administered to a patient already suffering from a disease or condition, in an amount sufficient to cure or at least partially arrest the symptoms of the disease or condition. Amounts effective for this use will depend on the severity and course of the disease or condition, previous therapy, the patient's health status, weight, and response to the drugs, and the judgment of the treating physician. It is considered well within the skill of the art for one to determine such therapeutically effective amounts by routine experimentation (including, but not limited to, a dose escalation clinical trial).

In prophylactic applications, compositions containing the compounds described herein are administered to a patient susceptible to or otherwise at risk of a particular disease, disorder or condition. Such an amount is defined to be a “prophylactically effective amount or dose.” In this use, the precise amounts also depend on the patient's state of health, weight, and the like. It is considered well within the skill of the art for one to determine such prophylactically effective amounts by routine experimentation (e.g., a dose escalation clinical trial). When used in a patient, effective amounts for this use will depend on the severity and course of the disease, disorder or condition, previous therapy, the patient's health status and response to the drugs, and the judgment of the treating physician.

In the case wherein the patient's condition does not improve, upon the doctor's discretion the administration of the compounds may be administered chronically, that is, for an extended period of time, including throughout the duration of the patient's life in order to ameliorate or otherwise control or limit the symptoms of the patient's disease or condition.

The amount of a given agent that will correspond to such an amount will vary depending upon factors such as the particular compound, disease or condition and its severity, the identity (e.g., weight) of the subject or host in need of treatment, but can nevertheless be routinely determined in a manner known in the art according to the particular circumstances surrounding the case, including, e.g., the specific agent being administered, the route of administration, the condition being treated, and the subject or host being treated. In general, however, doses employed for adult human treatment will typically be in the range of 0.02-5000 mg per day, preferably 1-1500 mg per day. The desired dose may conveniently be presented in a single dose or as divided doses administered simultaneously (or over a short period of time) or at appropriate intervals, for example as two, three, four or more sub-doses per day.

The pharmaceutical composition described herein may be in unit dosage forms suitable for single administration of precise dosages. In unit dosage form, the formulation is divided into unit doses containing appropriate quantities of one or more compound. The unit dosage may be in the form of a package containing discrete quantities of the formulation. Non-limiting examples are packaged tablets or capsules, and powders in vials or ampoules. Aqueous suspension compositions can be packaged in single-dose non-reclosable containers. Alternatively, multiple-dose reclosable containers can be used, in which case it is typical to include a preservative in the composition. By way of example only, formulations for parenteral injection may be presented in unit dosage form, which include, but are not limited to ampoules, or in multi-dose containers, with an added preservative.

In some embodiments, the daily dosages appropriate for the compounds described herein to alleviate the symptoms described herein are from about 0.001 to about 50 mg/kg per body weight. In other embodiments, the daily dosages appropriate for the compounds described herein are from about 0.01 to about 20 mg/kg per body weight. In further embodiments, the daily dosages appropriate for the compounds described herein described herein are from about 0.01 to about 2.5 mg/kg per body weight. An indicated daily dosage in the larger mammal, including, but not limited to, humans, is in the range from about 0.5 mg to about 100 mg, conveniently administered in divided doses, including, but not limited to, up to four times a day or in extended release form. Suitable unit dosage forms for oral administration include from about 1 to 50 mg active ingredient. The foregoing ranges are merely suggestive, as the number of variables in regard to an individual treatment regime is large, and considerable excursions from these recommended values are not uncommon. Such dosages may be altered depending on a number of variables, not limited to the activity of the compound used, the disease or condition to be treated, the mode of administration, the requirements of the individual subject, the severity of the disease or condition being treated, and the judgment of the practitioner.

Combination Treatments

The compositions and methods described herein may also be used in conjunction with other well known therapeutic reagents that are selected for their particular usefulness against the condition that is being treated. In general, the compositions described herein and, in embodiments where combinational therapy is employed, other agents do not have to be administered in the same pharmaceutical composition, and may, because of different physical and chemical characteristics, have to be administered by different routes. The determination of the mode of administration and the advisability of administration, where possible, in the same pharmaceutical composition, is well within the knowledge of the skilled clinician. The initial administration can be made according to established protocols known in the art, and then, based upon the observed effects, the dosage, modes of administration and times of administration can be modified by the skilled clinician.

In addition, the compounds described herein also may be used in combination with procedures that may provide additional or synergistic benefit to the patient. By way of example only, patients are expected to find therapeutic and/or prophylactic benefit in the methods described herein, wherein pharmaceutical composition of a compound disclosed herein and /or combinations with other therapeutics are combined with genetic testing to determine whether that individual is a carrier of a mutant gene that is known to be correlated with certain diseases or conditions.

Kits/Articles of Manufacture

For use in the therapeutic applications described herein, kits and articles of manufacture are also described herein. Such kits can include a carrier, package, or container that is compartmentalized to receive one or more containers such as vials, tubes, and the like, each of the container(s) including one of the separate elements to be used in a method described herein. Suitable containers include, for example, bottles, vials, syringes, and test tubes. The containers can be formed from a variety of materials such as glass or plastic.

### EXAMPLES

The person skilled in the art may further appreciate various aspects and advantages of the present disclosure upon review of the following illustrative and non-limiting examples:

General Procedure for the Preparation of Esters of Alkylcarbamic Acids

To a stirred suspension or solution of a hydroxy-containing compound, in ethanol or acetonitrile, was added triethylamine and an alkylisocyanate (or alkylisothiocyanate). The reaction was stirred at room temperature for 2-16 hours. An additional amount of the alkylisocyanate (or alkylisothiocyanate) is added if needed. The reaction mixture is cooled and the product is filtered. Otherwise the reaction mixture is purified by methods known in the art, such as, but not limited to, chromatography, filtration, distillation, recrystallization, and combinations thereof.

### Example 1

### Synthesis of 4-acetamidophenyl cyclohexylcarbamate

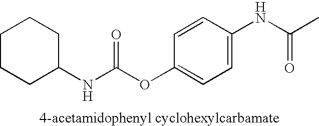

To a stirred suspension of acetaminophen in acetonitrile was added triethylamine (TEA) (1.1 equivalents) and cyclohexyl isocyanate (1.0 equivalent). The reaction mixture was stirred at room temperature for 16 hours. An additional amount of cyclohexyl isocyanate (0.2 equivalents) was added to the reaction mixture. The reaction mixture was stirred for an additional 16 hours. The product was isolated by cooling the reaction mixture with an ice bath and filtering. The filtercake was washed with cold acetonitrile. A white chrystalline solid was obtained after drying at 25-30° C.

### Example 2

### Synthesis of 4-acetamidophenyl cyclohexylmethylcarbamate

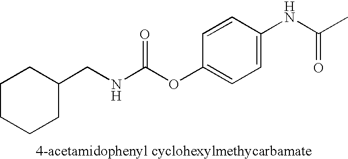

To a stirred suspension of acetaminophen in acetonitrile was added triethylamine (TEA) (1.1 equivalents) and cyclohexanemethyl isocyanate (1.0 equivalent). The reaction mixture was stirred at room temperature for 16 hours. An additional amount of cyclohexanemethyl isocyanate (0.2 equivalents) was added to the reaction mixture. The reaction mixture was stirred for an additional 16 hours. The product was isolated by cooling the reaction mixture with an ice bath and filtering. The filtercake was washed with cold acetonitrile. A white crystalline solid was obtained after drying at 25-30° C.

Methods of Screening Compound for FAAH Inhibitory Activity

Methods of screening compounds for fatty acid amide hydrolase (FAAH) inhibitory activity are well known to one of ordinary skill in the art. Methods of screening compounds for FAAH inhibitory activity in vivo including consequential increases in endogenous fatty acid amide levels or activities are known to one of ordinary skill in the art. Such methods are disclosed in: Quistand et al. Toxicology and Applied Pharmacology 179: 57-63 (2002); Quistand et al. Toxicology and Applied Pharmacology 173, 48-55 (2001); Boger et al., Proc. Natl. Acad. Sci. U.S.A. 97, 5 044-49 (2000); Ramarao M K, et al. 2005. Anal Biochem. 343: 143-5 1. U.S. Pat. No. 6,096,784., PCT Publication WO 98/24396, U.S. Pat. Publication No. 2004/0127518, and PCT Publication WO 04/033422., and Cravatt et al. Proc. Natl. Acad. Sci. U.S.A. 98, 9371-9376 (2001).

### Example 3

### Compound Screening for Inhibition of FAAH Activity—FAAH LC-MS/MS Screening Assay

In one embodiment, inhibition of FAAH activity is determined using LC-MS/MS. The following are combined in a 5-mL glass tube: anandamide (5 μL of 200 ug/mL), 960 μL of 50 mM ammonium phosphate buffer (pH 7.4) containing 0.125% BSA (w/v), 10 μL of DMSO without (control) or with a FAAH inhibitor (1 μg/mL), and 25 μL of human liver microsomes (31.3 μg). Prior to incubation, a 100 μL aliquot is transferred to a 96-well plate containing 0.25 mL of acetonitrile and D₄ (deuterated) anandamide (0.2 μM). Each 5-mL tube is capped and placed in a shaking water bath maintained at 37° C. for 60 minutes. After a 60 minute incubation, a second 100 μL aliquot is transferred to a 96-well plate as performed earlier. The 96-well plate is then capped, vortex mixed, and placed on an HPLC for liquid chromatography/tandem mass spectrometry (LC/MS/MS) analyses. HPLC is carried out on a Waters 2790 Alliance system (Milford, Mass.). Separation was performed on a Phenomenex C18 column (2 mm×50 mm, 4 μ; Torrance, Calif.) using an isocratic mobile phase of acetonitrile:water:formic acid (80:20:0.1, v/v/v) at a flow rate of 0.3 mL min⁻¹ and a column temperature of 45° C. The HPLC system was interfaced with a Micromass Ultima tandem MS (Beverly, Mass.). The samples are analyzed using an electrospray probe in the positive ionization mode with the cone voltage set at 40 V and capillary at 3.2 kV. The source and desolvation temperature settings are 130° C. and 500° C., respectively. The voltage of the CID chamber is set at −20 eV. Multiple reaction monitoring is used for the detection of anandamide as [M+H] (m/z 348>62) and D₄ anandamide (internal standard) as [M+H] (m/z 352>66). An area ratio response (anandamide area response/D₄ anandamide area response) was determined for each sample. Percent anandamide hydrolysis of each sample is determined by the following equation, [(T=0 response)−(T=60 response)/T=0 response]* 100. The percent hydrolysis normalized to control is determined by dividing the % hydrolysis of test sample by the % hydrolysis of the control sample. As an example, 4-acetamidophenyl cyclohexylcarbamate and 4-acetamidophenyl cyclohexylmethylcarbamate, at a concentration of 30 nM, allowed only 56% and 37% anandamide hydrolysis, respectively. The compound 3′-carbamoylbiphenyl-3-yl cyclohexylcarbamate allowed 30% anandamide hydrolysis at the same concentration in the same assay.

For determining IC₅₀ values for candidate FAAH inhibitor compounds, the above method is used with an adjusted FAAH inhibitor concentration. In the IC₅₀ assay, the FAAH inhibitor is added at a concentration range of approximately 3 μM to 0.03 nM. The final calculation of IC₅₀ is determined by first transforming the concentrations by “X=log(X)” and then analyzing the data with a sigmoidal dose-response curve (no constraints) using GraphPad Prism version 4.00 for Windows (GraphPad Software, San Diego Calif. USA, www.graphpad.com). The compound 4-acetamidophenyl cyclohexylmethylcarbamate had an IC₅₀ of 14.6 nM and the compound 3′-carbamoylbiphenyl-3-yl cyclohexylcarbamate had an IC₅₀ of 2.5 nM.

### Example 4

### Compound Screening for Inhibition of FAAH Activity—FAAH Fluorescent Screening Assay

To a black 96-well plate (Nunc, cat #267342) is added 180 μL of arachidonyl 7-amino,4-(AAMCA, 3 μM), 20 μL of a FAAH inhibitor (0.05 μg/mL in DMSO) and 50 μL of human liver microsomes (0.25 mg/mL). The diluent for the AAMCA and human liver microsomes is fatty acid free BSA (1.4 mg/mL) in HEPES/EDTA (50 mM/1 mM) at pH 7.4. The plate is read at excitation 355 nm and emission 460 nm at T=0 on a fluorescence plate reader (SpectraMax GeminiXS, Molecular Devices) and incubated for 30 minutes at 37° C. After the 30 minute incubation, the plate is read a final time and % hydrolysis (normalized to control) was determined. The calculation for % hydrolysis is [(T=30−T=0)/T=0]*100. The percent hydrolysis normalized to control is determined by dividing the % hydrolysis of test sample by the % hydrolysis of the control sample (DMSO).

### Example 5

### Compound Screening for Inhibition of FAAH Activity—Screening for In Vivo FAAH Inhibition in Rats

Potential FAAH inhibitors are formulated for oral (p.o.), intraperitoneal (i.p.) or intravenous (i.v.) delivery to rats. Formulated compounds are administered and the animals were sacrificed at pre-determined time points post dose. At sacrifice, blood samples are collected into EDTA plasma tubes and whole brains were snap frozen in liquid nitrogen. EDTA plasma was isolated from blood samples after centrifugation. Brain and plasma samples are stored at −80° C. prior to analysis. All samples (brain and plasma) are analyzed for the concentrations of test compound (FAAH inhibitor), metabolites of the test compound (such as, for example, acetaminophen, propofol, NSAID, or NSAID metabolite) and endogenous fatty acid ethanolamide levels (including anandamide, oleoylethanolamide, and palmitoylethanolamide) by LC-MS/MS according to the invention. Levels of these compounds are compared across time points to determine pharmacokinetic properties of the test compound and partial pharmacological effects of inhibiting FAAH activity (including changes of fatty acid ethanolamide levels).

In one embodiment, additional tissues and fluid samples can be collected at sacrifice. In one embodiment, FAAH activity can also be determined in fluid and tissues samples according to the methods disclosed or according to methods known in the art. In one embodiment, metabolites of the test compounds can be determined in fluid and tissue samples. In another embodiment, the fluid and tissue samples are analyzed for content of acetaminophen, propofol, NSAID or NSAID metabolite.

### Example 6

### Determination of Pharmacokinetics of 4-acetamidophenyl Cyclohexylmethylcarbamate

The pharmacokinetic properties of 4-acetamidophenyl cyclohexylnethylcarbamate were assessed in rats following oral administration as a suspension. To test the oral bioavailability of 4-acetamidophenyl cyclohexylmethylcarbamate, a suspension of 4-acetamidophenyl cyclohexylmethylcarbamate was prepared for oral administration as a 100 mg/mL suspensions in 0.5% sodium carboxymethyl cellulose, 0.5% simethicone, and 0.4% Polysorbate 80 in water (w/v). The suspension of 4-acetamidophenyl cyclohexylmethylcarbamate was administered to rats at a dose of 10 mg/kg of 4-acetamidophenyl cyclohexylmethylcarbamate via oral gavage. Blood samples were extracted from the rats. Samples were analyzed for concentrations of OEA, PEA, AEA, 4-acetamidophenyl cyclohexylmethylcarbamate and acetaminophen (in-vivo metabolite). The results are presented in Table 1. Baseline levels of OEA and PEA were 4.18 and 4.17 ng/mL, respectively.

TABLE 1
PK properties of 4-acetamidophenyl cyclohexylmethylcarbamate in
rats following oral administration.

| | | Ace- | | | |
|---|---|---|---|---|---|
| | 4-acetamidophenyl | tamin- | | | |
| | cyclohexylmethylcarbamate | ophen | OEA | PEA | AEA |
| AUC | 6775 | 170 | 40 | 43 | 0 |
| (ng * hr/mL) | | | | | |
| Cmax | 7742 | 62 | 7.55 | 8.85 | 0 |
| (ng/mL) | | | | | |
| Tmax (hr) | 0.5 | 0.5 | 1.5 | 1 | 0 |

Animal Models

Any of a variety of animal models can be used to test the compounds disclosed herein, such as, compounds of Formula (I), for their effectiveness in reducing inflammation and treating pain. Useful compounds can exhibit effectiveness in reducing inflammation or pain in one or more animal models.

Animal Models for Assessing Anti-Inflammatory Activity

### Example 7

### Carrageenan-Induced Foot Pad Edema Model

The model is described, for example, by Winter et al. (1962 Proc Soc Exp Biol Med 111 :544). Briefly, rats are fasted with free access to water for 17 to 19 hours before oral treatment with up to three doses of a test compound, indomethacin or celecoxib, or a control vehicle (1% methylcellulose in deionized water). One hour after the last treatment, paw edema is induced by injecting 0.05 ml of a 2% carrageenan solution into the left hindpaw. The left hindpaw volume of each rat is measured using a plethysmometer before oral treatment, at the time of carrageenan injection and at 1.5 h, 3 h, 4.5 h after the injection of carrageenan. The edema volume of each rat at each time point is expressed as the change from the volume at the time of oral treatment and the anti-inflammatory effect in treated groups is expressed as % inhibition compared to the vehicle only group 1.5 h, 3 h and 4.5 h after the carrageenan injection. The significance of the difference between in edema different groups is assessed by a one-way analysis of variance (ANOVA) followed by the non-paired Dunnett t test. In this model, hyperalgesic response and PGE₂ production can also be measured (Zhang et al. 1997 J Pharmacol and Exp Therap 283:1069).

### Example 8

### Complete Freund's Adjuvant (CFA) Induced Arthritis Model

In this model, arthritis is induced in groups of eight Lewis derived male rats weighing 160±10 g by injecting a well-ground suspension of killed Mycobacterium tuberculosis (0.3 mg in 0.1 mL of light mineral oil; Complete Freund's Adjuvant, CFA) into the subplantar region of the right hind paw on Day 1. Hind paw volumes are measured by water displacement on Days 0, 1 and 5 (right hind paw, with CFA), and on Days 0, 14 and 18 (left hind paw, without CFA); rats are weighed on Days 0 and 18. Test compounds, dissolved or suspended in 2% Tween 80, are prepared fresh daily and administered orally twice daily for 5 consecutive days (Day 1 through day 5) beginning one hour before injection of CFA. For CFA-injected vehicle control rats, the increase in paw volume on Day 5 relative to Day 1 (Acute Phase of inflammation) is generally between 0.7 and 0.9 mL; and, that on Day 18 relative to day 14 (Delayed Phase of inflammation) is generally between 0.2 and 0.4 mL. Thus, anti-inflammatory activity in this model may be denoted by values calculated during the Acute Phase as well as the Delayed Phase. Animals are also weighed on Day 0 and Day 18; CFA-injected vehicle control animals generally gain between 40 to 60 g body weight over this time period. A 30 percent or more reduction in paw volume relative to vehicle treated controls is considered of significant anti-inflammatory activity. The mean±SEM for each treatment group is determined and a Dunnett test is applied for comparison between vehicle and treated groups. Differences are considered significant at P<0.05. Polyarthritis of fore paw, tail, nose and ear can be scored visually and noted on the first day and final day, wherein positive (+) sign is for swelling response and negative (−) sign is normal. X-ray radiographies of the hindpaws can also be performed for further radiological index determination of arthritic symptoms. Hyperalgesia can also be measured in this model, allowing determination of analgesic effects of test compounds (Bertorelli et al. 1999 Brit J. Pharmacol 128:1252).

### Example 9

### Air-Pouch Model

This model is described by Masferrer et al. (1994 Proc Natl Acad Sci USA 91:3228). Briefly, male Lewis rats (175-200 g, Harlan Sprague-Dawley) are subcutaneously injected with 20 mL of sterile air into the intrascapular area of the back to create air cavities. An additional 10 mL of air is injected into the cavity every 3 days to keep the space open. Seven days after the initial air injection, 2 mL of a 1% solution of carrageenan dissolved in sterile saline is injected directly into the pouch to produce an inflammatory response. In treated and untreated animals, the volume of exudate is measured and the number of leukocytes present in the exudate is determined by Wright-Giemsa staining. In addition, PGE₂ and 6-keto-PGF_{1α} are determined in the pouch exudates from treated and untreated animals by specific ELISAs (Cayman Chemicals, Ann Arbor, Mich.).

Animal Models for Assessing Analgesic Activity

### Example 10

### Carrageenan-Induced Thermal Hyperalgesia

This model is described by Hargreaves et al. (1988 Pain 32:77). Briefly, inflammation is induced by subplantar injection of a 2% carrageenan suspension (0.1 mL) into the right hindpaw. Three hours later, the nociceptive threshold is evaluated using a thermal nociceptive stimulation (plantar test). A light beam (44% of the maximal intensity) is focused beneath the hindpaw and the thermal nociceptive threshold is evaluated by the paw flick reaction latency (cut-off time: 30 sec). The pain threshold is measured in ipsilateral (inflamed) and in contralateral (control) hindpaws, 1 hour after the oral treatment with the test compound or a control. The results can be expressed as the nociceptive threshold in seconds (sec) for each hindpaw and the percentage of variation of the nociceptive threshold (mean±SEM) for each rat from the mean value of the vehicle group. A comparison of the nociceptive threshold between the inflamed paw and the control paw of the vehicle-treated group is performed using a Student's t test, a statistically significant difference is considered for P<0.05. Statistical significance between the treated groups and the vehicle group is determined by a Dunnett's test using the residual variance after a one-way analysis of variance (P<0.05) using SigmaStat Software.

### Example (10a)

### Complete Freund's Adjuvant (CFA)-Induced Mechanical Hyperalgesia

In this rat model of inflammatory pain, inflammation is induced by intraplantar injection of complete Freund's adjuvant (CFA) into the hind paw. The associated mechanical hyperalgesia is quantified by measuring the reduction in paw withdrawal threshold (PWT) to a mechanical compression of the paw.

Experiments were conducted using male Wistar rats weighing 130-180 g. In brief, the rats' paw withdrawal threshold (PWT) to a mechanical compression of the hind paw was measured (baseline reading) using a Randall-Sellito apparatus (Ugo Basile). A cut-off of 200 g was employed to minimise tissue damage to the paw. The animals were then briefly anaesthetised with isoflurane (˜2%) and complete Freund's adjuvant (0.1 ml per paw) was injected subcutaneously (s.c.) into the plantar surface of the left hind paw. The animals were then returned to their home cage and left for the inflammation to develop.

Twenty four hours after CFA injection, PWT's were re-measured (0 min); only those animals developing hyperalgesia (>20 g decrease in PWT compared to their baseline readings) were included for drug assessment. Rats were then dosed orally with either vehicle or test compound. Readings were then made at 2 h post drug administration.

Oral administration of compounds of the invention reversed the mechanical hyperalgesia induced 24 h after intraplantar injection of CFA in rats. As an example, compound 4-acetamidophenyl cyclohexylmethylcarbamate (60 mg/kg, p.o.) elicited complete reversal of mechanical hyperalgesia in this model.

### Example 11

### Phenylbenzoquinone-Induced Writhing Model

This model is described by Siegmund et al. (1957 Proc Soc Exp Bio Med 95:729). Briefly, one hour after oral dosing with a test compound, morphine or vehicle, 0.02% phenylbenzoquinone (PBQ) solution (12.5 mL/kg) is injected by intraperitoneal route into the mouse. The number of stretches and writhings are recorded from the 5th to the 10th minute after PBQ injection, and can also be counted between the 35th and 40th minute and between the 60th and 65th minute to provide a kinetic assessment. The results are expressed as the number of stretches and writhings (mean±SEM) and the percentage of variation of the nociceptive threshold calculated from the mean value of the vehicle-treated group. The statistical significance of any differences between the treated groups and the control group is determined by a Dunnett's test using the residual variance after a one-way analysis of variance (P<0.05) using SigmaStat Software.

### Example 12

### Kaolin-Induced Arthritis Model

This model is described by Hertz et al. (1980 Arzneim Forsch 30:1549). Briefly, arthritis is induced by injection of 0.1 mL of kaolin suspension into the knee joint of the right hind leg of a rat. Test compounds are administered subcutaneously after 15 minutes and again after two hours. Reference compounds can be administered orally or subcutaneously. Gait is assessed every hour from 1.5 hours to 5.5 hours after treatment and is scored as follows: normal gait (0), mild disability (1), intermittent raising of paw (2), and elevated paw (3). Results are expressed as the mean gait score (mean±SEM) calculated from individual values at each time point and the percentage of variation of the mean score calculated from the mean value of the vehicle-treated group at 4.5 hours and 5.5 hours after treatment. The statistical significance of differences between the treated groups and the vehicle-treated group is determined by a Dunnett's test using the residual variance after a one-way analysis of variance (P<0.05) at each time point.

### Example 13

### Peripheral Mononeuropathy Model

This model is described by Bennett et al. (1988 Pain 33:87) and can be used to assess anti-hyperalgesic effect of an orally administered test compound in a model of peripheral mononeuropathy. The effect of the test substance can be compared to a no treatment control or reference substance, e.g., morphine. Peripheral mononeuropathy is be induced by loose ligation of the sciatic nerve in anaesthetized male Sprague Dawley rats (pentobarbital; 45 mg/kg by intraperitoneal route). Fourteen days later, the nociceptive threshold is evaluated using a mechanical nociceptive stimulation (analgesimeter paw pressure test; Ugo Basile, Italy). The test and reference compounds and the vehicle are orally administered (10 mL/kg carried 1% methylcellulose). Increasing pressure is applied to the hindpaw of the animal until the nociceptive reaction (vocalization or paw withdrawal) is reached. The pain threshold (grams of contact pressure) is measured in ipsilateral (injured) and in contralateral (non injured) hindpaws, 60 minutes after treatment. The results are expressed as: the nociceptive threshold (mean±SEM) in grams of contact pressure for the injured paw and for the non-injured paw (vehicle-treated group) and the percentage of variation the nociceptive threshold calculated from the mean value of the vehicle-treated group. A comparison of the nociceptive threshold between the non injured paw and the injured paw of the vehicle-treated group is performed using a Student's t test. The statistical significance of the difference between the treated groups and the vehicle group is determined for the injured hindpaw by a Dunnett's test using the residual variance after a one-way analysis of variance (P<0.05) using SigmaStat Software (SigmaStat.RTM. v. 2.0.3 (SPSS Science Software, Erkrath GmbH)).

### Example 14

### Chung Rat Model of Peripheral Neuropathy

In one embodiment, the effectiveness of a compound provided herein in alleviating neuropathic pain is demonstrated using the well-recognized Chung rat model of peripheral neuropathy. In the Chung rat model, spinal nerve partial ligation of left spinal nerves L-5 and L-6 produces a long-lasting hypersensitivity to light pressure on the affected left foot. The hypersensitivity is similar to the pain experienced by humans with the neuropathic condition of causalgia (Kim and Chung, Pain 50:355-363 (1992), which is incorporated herein by reference).

### Example 15

### Diabetic Neuropathy Paw Pressure Test

Complete protocol details can be found in Rakieten et al. (1963 Cancer Chemother Rep 29:91). Briefly, diabetes is induced by intraperitoneal injection of streptozotocin in rats. Three weeks later, the nociceptive threshold is measured using the paw pressure test to assess hyperalgesia. Test compound or controls are administered intraperitoneally 30 minutes prior to pain measurement.

### Example 16

### Acetic Acid Writhing Test

Briefly, a test compound is administered orally one hour before intraperitoneal injection of acetic acid (0.5%, 10 ml/kg) in rats. Reduction in the number of writhes by 50 percent or more (≧50) per group of animals observed during the 5 to 11 minute period after acetic acid administration, relative to a vehicle treated control group, indicates possible analgesic activity. This assay is based on that described in Inoue, K. et al. (1991 Arzneim. Forsch./Drug Res. 41: 235).

### Example 17

### Formalin Test

Complete protocol details can be found in Hunskaar et al. (1985 Neurosci. Meth. 14:69). Briefly, 30 minutes after intraperitoneal administration of a test compound or a control, 20 μL of a 5% formalin solution is injected by subplantar route into the right hindpaw of the rat. Hindpaw licking time is recorded during the early phase and the later phase after formalin injection.

### Example 18

### Tail Flick Test

Complete protocol details can be found in D'Amour and Smith (1941 J Pharmacol. Exp Ther. 72:74). Briefly, 30 minutes after intraperitoneal administration of a test compound or a control, a light beam is focused onto the tail of the rat. The nociceptive reaction latency, characterized by tail withdrawal, is recorded. The cutoff time is set to 15 seconds.

### Example 19

### Tail Immersion Test

In this test the tail of the rat is immersed into a 50-60° C. water bath. The nociceptive reaction latency, characterized by tail withdrawal, is measured (Haubrich et al. 1990 J Pharmacol Exp Ther 255:511 and Lichtman et al. 2004 Pain 109:319).

### Example 20

### Hot Plate Test

Complete protocol details can be found in Eddy et al. (1950 J Pharmacol. Exp. Ther. 98:121). Briefly, 30 minutes after intraperitoneal administration of a test compound or a control, the mouse is placed on a metallic hot plate maintained at 52° C. The nociceptive reaction latency, characterized by a licking reflex of the forepaws or by a jumping off the hot plate is recorded. The cut-off time is set to 30 seconds.

Assays for Assessing Anxiolytic Activity

Compounds of the invention that modulate FAAH activity, and thus fatty acid amide levels, may also have anxiolytic activity. Animal models to assess anxiolytic activity include:

### Example 21

### Elevated Plus Maze

The elevated plus maze consists of four maze arms that originate from a central platform, effectively forming a plus sign shape as described in van Gaalen and Steckler (2000 Behavioural Brain Research 115:95). The maze can be made of plexiglas and is generally elevated. Two of the maze arms are unwalled (open) and two are walled (closed). The two open arms are well lit and the two enclosed arms are dark (Crawley 2000 What's Wrong With My Mouse?: Behavioral Phenotyping of Transgenic and Knockout Mice. Wiley-Liss, N.Y.). The test is premised on the naturalistic conflict between the tendency of an animal to explore a novel environment and the aversive properties of a brightly lit, open area (Pellow et al. 1985 J. Neuroscience Methods. 14:149).

Complete protocol details can be found in Fedorova et al. (2001 J. Pharm. Exp. Ther. 299: 332). Briefly, 15 minutes following intraperitoneal administration of test compound or control, an animal is placed individually on the central platform, facing one of the open arms opposite to the observer. The number of open and closed arm entries, and the time spent in the different compartments of the maze by the animal (central platform, open and closed arms) is scored (as described in Gaalen et al. (supra)). An arm visit is recorded when an animal moves all four paws into the arm as described in Simonin et al. (1998 EMBO J. 17: 886). Behavior is scored by an observer and/or via a video camera over a 5-minute test session. A greater amount of time spent or entries made by the animal in the open versus the closed arms is an indicator of anxiolytic activity.

### Example 22

### Elevated Zero Maze

The elevated zero maze is a modification of the elevated plus maze. The elevated zero maze consists of a plexiglas apparatus in the shape of a circle (i.e., a circular runway of 46 cm diameter and 5.5 cm runway width) with two open and two wall-enclosed sectors of equal size. It is elevated up to a meter above the ground. This apparatus is described in Simonin et al. (supra) and Crawley (supra).

Complete protocol details can be found in Kathuria et al (2003 Nature Medicine 9: 76). Briefly, 30 minutes following intraperitoneal administration of test compound or control, an animal is placed on one open sector in front of an enclosed sector. Time in a new sector is recorded as entry with all four paws. Behavior will be scored by an observer and/or via a video camera over a 5-minute test session. A greater amount of time spent or entries made by the animal in the open versus the walled sector is an indicator of anxiolytic activity.

### Example 23

### Isolation-Induced Ultrasonic Emission Test

In another animal model, the isolation-induced ultrasonic emission test, compounds provided herein are tested for their anti-anxiety effects. The isolation-induced ultrasonic emission test measures the number of stress-induced vocalizations emitted by rat pups removed from their nest (Insel, T. R. et al, Pharmacol. Biochem. Behav., 24, 1263-1267 (1986); Miczek, K. A. et al, Psychopharmacology, 121, 38-56 (1995); Winslow, J. T. et al., Biol Psychiatry, 15, 745-757 (1991); U.S. Pat. No. 6,326,156).

Assays for Assessing Antinociception Mechanism

Compounds can be tested to determine if they influence pathways involved in nociception. The results of such assays can be used to investigate the mechanism by which a test compound mediates its antinociceptive effect.

### Example 24

### Elevation of 3α,5α-THP

3α-hydroxy-5α-pregan-20-one (3α,5α-THP or allopregnanolone) is a pregnane steroid that acts as an agonist of the inhibitory GABA_{A} receptor subtype and is known to have both anxiolytic and analgesic effects in a variety of animal systems, with supportive evidence for a similar role in humans. Thus, compounds that elevate 3α,5α-THP may have an antinociceptive effect. The level of 3α,5α-THP in the brain of animals treated with a test compound can be measured as described by VanDoren et al. (1982 J Neuroscience 20:200). Briefly, steroids are extracted from individual cerebral cortical hemispheres dissected in ice-cold saline after euthanasia. Cortices are frozen at −80° C. until use. Samples are digested in 0.3 N NaOH by sonication and extracted three times in 3 mL aliquots of 10% (v/v) ethyl acetate in heptane. The aliquots are combined and diluted with 4 mL of heptane. The extracts are applied to solid phase silica columns (Burdick & Jackson, Muskegon, Mich.), washed with pentane, and steroids of similar polarity to 3α,5α-THP are eluted off of the column by the addition of 25% (v/v) acetone in pentane. The eluant is then dried under N₂ and steroids are redissolved in 20% (v/v) isopropanol RIA buffer (0.1 M NaH₂PO₄, 0.9 M NaCl, 0.1% w/v BSA, pH 7.0). Extraction efficiency is determined in 50 82 L of the redissolved extract by liquid scintillation spectroscopy and the remaining sample is used in the determination of 3α,5α-THP by radioimmunoassay. Reconstituted sample extracts (75 μL) and 3α,5α-THP standards (5-40,000 pg in 6.25% v/v ethanol, 31% v/v isopropyl alcohol in RIA buffer) are assayed in duplicate by the addition of 725 μL of RIA buffer, 100 μL of [³H] 3α,5α-THP (20,000 dpm), and 100 μL of anti-3α,5α-THP antibody. Total binding is determined in the absence of unlabeled 3α,5α-THP, and nonspecific binding is determined in the absence of antibody. The antibody-binding reaction is allowed to equilibrate for 120 min at room temperature and is terminated by cooling the mixture to 4° C. Bound 3α,5α-THP is separated from unbound 3α,5α-THP by incubation with 300 μL of cold dextran coated charcoal (DCC; 0.04% dextran, 0.4% powdered charcoal in double-distilled H₂O) for 20 min. DCC is removed by centrifugation at 2000>g for 10 min. Bound radioactivity in the supernatant is determined by liquid scintillation spectroscopy. Sample values are compared to a concurrently run 3α,5α-THP standard curve and corrected for extraction efficiency.

### Example 25

### Evaluation of Anti-Depressive Effects

In one embodiment, compounds provided herein, such as, for example, compounds of Formula (I), are evaluated for anti-depressive effects in animal models. The chronic mild stress induced anhedonia model is based on the observation that chronic mild stress causes a gradual decrease in sensitivity to rewards, for example consumption of sucrose, and that this decrease is doses-dependent and reversed by chronic treatment with antidepressants. The method has previously been described by Willner, Paul, Psychopharmacology, 1997, 134, 319-329.

Another test for antidepressant activity is the forced swimming test (Nature 266, 730-732, 1977). In this test, animals are administered the compound preferably by the intraperitoneal route or by the oral route 30 or 60 minutes before the test. The animals are placed in a crystallizing dish filled with water and the time during which they remain immobile is clocked. The immobility time is then compared with that of the control group treated with distilled water. Imipramine (25 mg/kg) may be used as the positive control. The antidepressant compounds decrease the immobility time of the mice thus immersed.

Another test for antidepressant activity is the caudal suspension test on the mouse (Psychopharmacology, 85, 367-370, 1985). In this test, animals are preferably treated with a compound provided here, such as, for example, a compound of Formula (I), by the intraperitoneal route or by the oral route 30 minutes to 6 hours before the test. The animals are then suspended by the tail and their immobility time is automatically recorded by a computer system. The immobility times are then compared with those of a control group treated with vehicle. Imipramine (25 mg/kg) may be used as the positive control. Antidepressant compounds decrease the immobility time of the mice.

Antidepressant effects of the compounds provided herein, such as, for example, compounds of Formula (I), can be tested in the DRL-72 TEST. This test, carried out according to the protocol of Andrews et al “Effects of imipramine and mirtazapine on operant performance in rats” Drug Development Research 32, 5 8-66 (1994), gives an indication of antidepressant-like activity. The effects of the compounds provided herein also may be examined in serotonin disorders and bipolar disorders, such as described in U.S. Pat. Nos. 6,403,573 and 5,952,315, incorporated herein by reference.

### Example 26

### Evaluation of Anticovulsant Effects

In another embodiment, compounds provided herein are examined for anticonvulsant activity in animal models, as described in U.S. Pat. Nos. 6,309,406 and 6,326,156.

### Example 27

### Effects of Compounds on Appetite Behavior

In one embodiment, compounds provided herein are administered to a rat in order to measure the effect on appetite behavior. The effect of the administered compound is assessed by examining the intake of a sucrose solution by the rat. This method is taught in W. C. Lynch et al., Physiol. Behav., 1993, 54, 877-880. Male Sprague-Dawley rats weighing about 190 g to about 210 g are under a normal light cycle (from 7 am to 7 pm) and receive water and food ad libitum. For 6 days, between 11 am and 3 pm, the food and the water bottles are withdrawn and the rats are given a 5% sucrose solution to drink. Rats drinking less than 3 g of sucrose solution are eliminated. On the seventh day the test is carried out according to the following procedure: 9 am: withdrawal of food, 10 am: administration of either a compound of Formula (I) or vehicle to the test animals; 11 am=T0: introduction of bottles containing a weighed sucrose solution; T0+1 hour, T0+2 hours, T0+3 hours, T0+4 hours: measurement of the sucrose consumption by weighing of the bottles. Followed by comparison of the experimental (administered a compound of Formula (I)) and control groups' intake of the sucrose solution. Animals can be, for example, obese or normal guinea pigs, rats, mice, or rabbits. Suitable rats include, for example, Zucker rats. Suitable mice include, for example, normal mice, ALS/LtJ, C3.5W-H-2b/SnJ, (NON/LtJ×NZO/H1J)FI, NZO/HIJ, ALR/LtJ, NON/LtJ, KK.Cg-AALR/LtJ, NON/LtJ, KK.CgAy/J, B6.HRS(BKS)-Cpefat/+, B6.129P2-GcktmlEfr, B6.V-Lepob, BKS.Cg-m+1+Leprdb, and C57BL/6J with Diet Induced Obesity.

In another test, the effect of a compound of the invention on the consumption of an alcohol solution can be shown in mice. For instance, male C 57 BL 6 mice are isolated on the day of their arrival in an animal housing under a reverse cycle (night from 10 am to 10 pm) with 2 bottles filled with water. After 1 week, one of the bottles of water is replaced with a bottle filled with a 10% alcohol solution for 6 hours of the test. Each day, 30 minutes before the bottle of alcohol is introduced, the mice are treated with a compound of the invention. The amounts of alcohol and water consumed are measured after 6 hours. The test is repeated for 4 days. The results for an experimental and a control or vehicle are compared.

### Example 28

### Cannabinoid Receptor Binding

Compounds may exert an antinociceptive effect via binding to either or both of the cannabinoid receptors CB₁ and CB₂. CB₁ is expressed in the brain (Matsuda et al. 1990 Nature 346:561), and CB₂ is expressed by macrophages and in the spleen (Munro et al. 1993 Nature 365:61). Both of these receptors have been implicated in mediating analgesic effects through binding of agonists (see, for example, Clayton et al. 2002 Pain 96:253). Thus, test compounds can be assayed to determine whether they bind to one or both human cannabinoid receptors. An assay for CB₁ binding is described by Matsuda et al. (supra). This assay employs recombinant cells expressing CB₁. Binding to CB₂ can be determined in the same manner using recombinant cells expressing CB₂. Briefly, to measure the ability of a test compound to bind to CB₁, the binding of a labelled CB₁ ligand, e.g., [³H]WIN 55212-2 (2 nM for CB₁ and 0.8 nM for CB₂) to membranes isolated from HEK-293 cells expressing recombinant CB₁ is measured in the presence and absence of a compound. Non-specific binding is separately determined in the presence of several-fold excess of unlabelled WIN 55212-2 (5 μM for CB₁ and 10 μM for CB₂). The specific ligand binding to the receptors is defined as the difference between the total binding and the non-specific binding determined in the presence of an excess of unlabelled WIN 55212-2. The IC₅₀ values and Hill coefficients (n_{H}) are determined by non-linear regression analysis of the competition curves using Hill equation curve fitting. The inhibition constants (Kᵢ) are calculated from the Cheng Prusoff equation (Kᵢ=IC₅₀/(1+(L/K_{D})), where L=concentration of radioligand in the assay, and K_{D}=affinity of the radioligand for the receptor).

### Example 29

### Pharmaceutical Compositions

### Example 29a

### Parenteral Composition

To prepare a parenteral pharmaceutical composition suitable for administration by injection, 100 mg of a water-soluble salt of a compound described herein is dissolved in DMSO and then mixed with 10 mL of 0.9% sterile saline. The mixture is incorporated into a dosage unit form suitable for administration by injection.

### Example 29b

### Oral Composition

To prepare a pharmaceutical composition for oral delivery, 100 mg of a compound described herein is mixed with 750 mg of starch. The mixture is incorporated into an oral dosage unit for, such as a hard gelatin capsule, which is suitable for oral administration.

### Example 29c

### Sublingual (Hard Lozenge) Composition

To prepare a pharmaceutical composition for buccal delivery, such as a hard lozenge, mix 100 mg of a compound described herein, with 420 mg of powdered sugar mixed, with 1.6 mL of light corn syrup, 2.4 mL distilled water, and 0.42 mL mint extract. The mixture is gently blended and poured into a mold to form a lozenge suitable for buccal administration.

### Example 29d

### Inhalation Composition

To prepare a pharmaceutical composition for inhalation delivery, 20 mg of a compound described herein is mixed with 50 mg of anhydrous citric acid and 100 mL of 0.9% sodium chloride solution. The mixture is incorporated into an inhalation delivery unit, such as a nebulizer, which is suitable for inhalation administration.

### Example 29e

### Rectal Gel Composition

To prepare a pharmaceutical composition for rectal delivery, 100 mg of a compound described herein is mixed with 2.5 g of methylcelluose (1500 mPa), 100 mg of methylparapen, 5 g of glycerin and 100 mL of purified water. The resulting gel mixture is then incorporated into rectal delivery units, such as syringes, which are suitable for rectal administration.

### Example 29f

### Topical Gel Composition

To prepare a pharmaceutical topical gel composition, 100 mg of a compound described herein is mixed with 1.75 g of hydroxypropyl celluose, 10 mL of propylene glycol, 10 mL of isopropyl myristate and 100 mL of purified alcohol USP. The resulting gel mixture is then incorporated into containers, such as tubes, which are suitable for topical administration.

### Example 29g

### Ophthalmic Solution Composition

To prepare a pharmaceutical ophthalmic solution composition, 100 mg of a compound described herein is mixed with 0.9 g of NaCl in 100 mL of purified water and filtered using a 0.2 micron filter. The resulting isotonic solution is then incorporated into ophthalmic delivery units, such as eye drop containers, which are suitable for ophthalmic administration.

The examples and embodiments described herein are for illustrative purposes only and various modifications or changes suggested to persons skilled in the art are to be included within the spirit and purview of this application and scope of the appended claims. All publications, patents, and patent applications cited herein are hereby incorporated by reference for all purposes.

What is claimed is:

1. A compound of Formula (I): 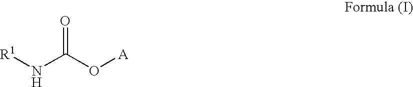
wherein:
R¹ is an optionally substituted group selected from among C₁-C₆ alkyl, C₃-C₉ cycloalkyl, and —C₁-C₄alkyl-(C₃-C₉cycloalkyl);
O-A is a deprotonated form of a hydroxy-containing compound selected from acetaminophen, propofol, an analgesic agent, an anti-inflammatory agent, an anti-pyretic agent, an NSAID, a metabolite of an analgesic agent, a metabolite of an anti-inflammatory agent, a metabolite of an anti-pyretic agent, and an NSAID metabolite; and pharmaceutically acceptable salts, pharmaceutically acceptable N-oxides, pharmaceutically active metabolites, pharmaceutically acceptable prodrugs, and pharmaceutically acceptable solvates thereof.

2. The compound of claim 1, wherein O-A is the deprotonated form of acetaminophen.

3. The compound of claim 1, wherein the compound of Formula (I) has a structure selected from:

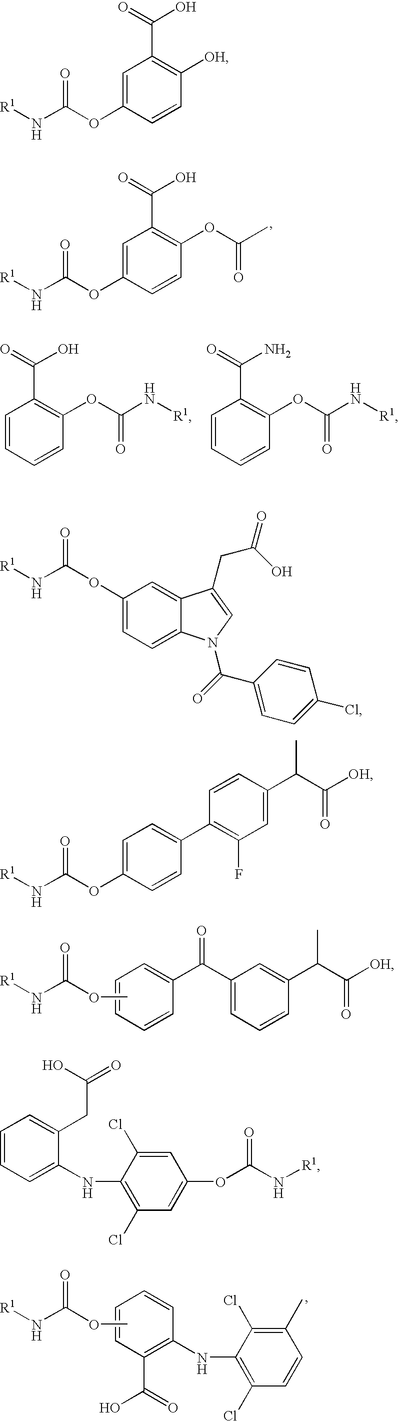

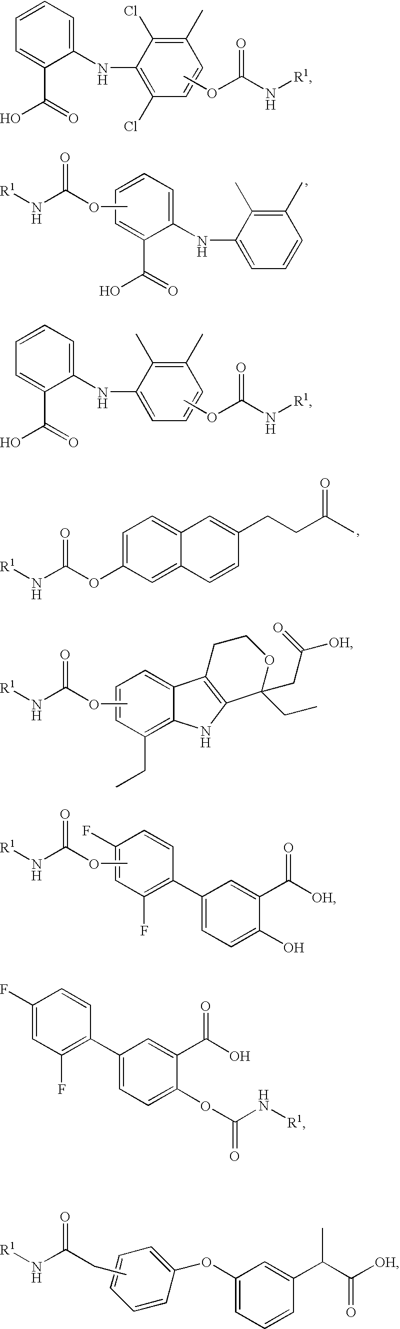

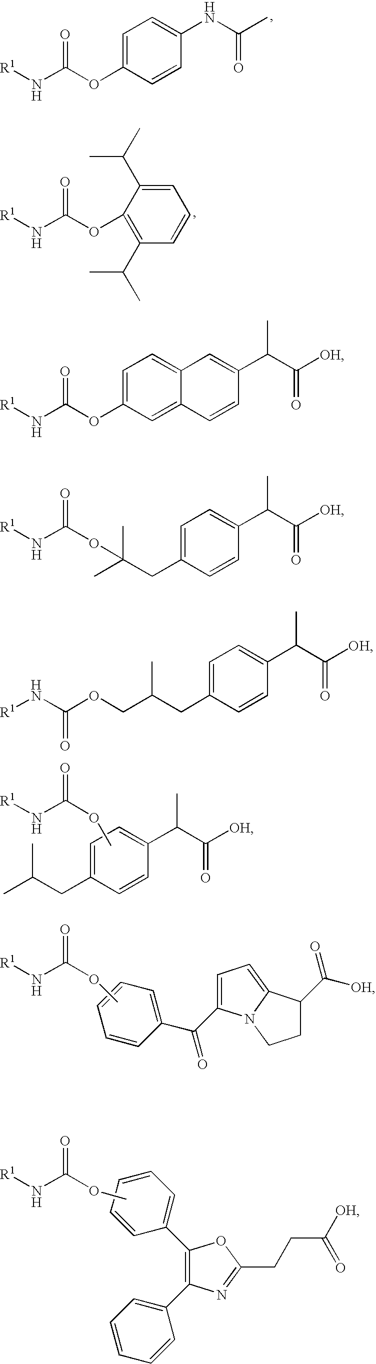

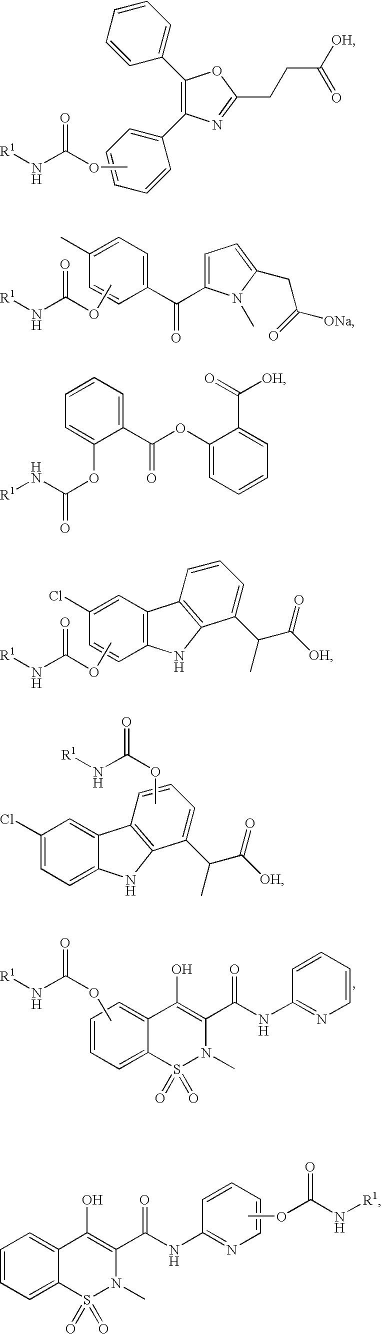

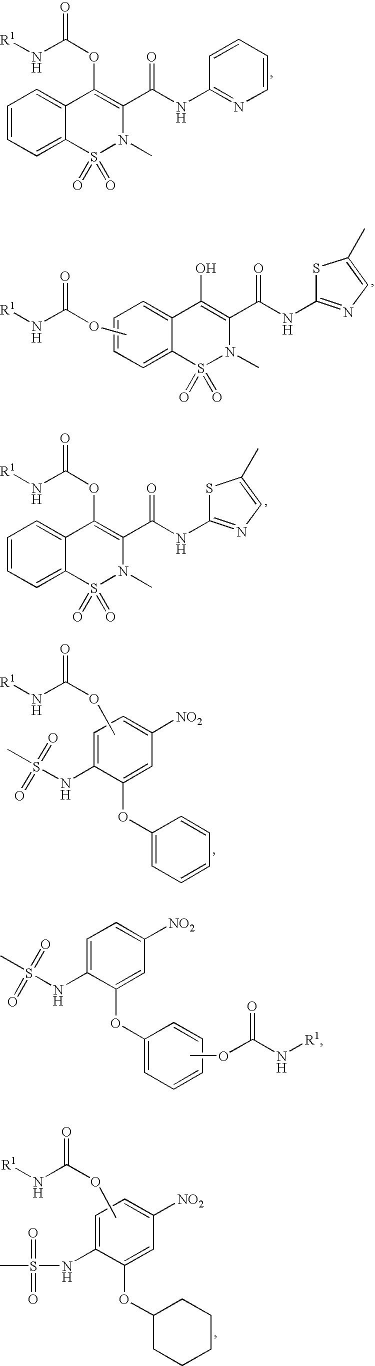

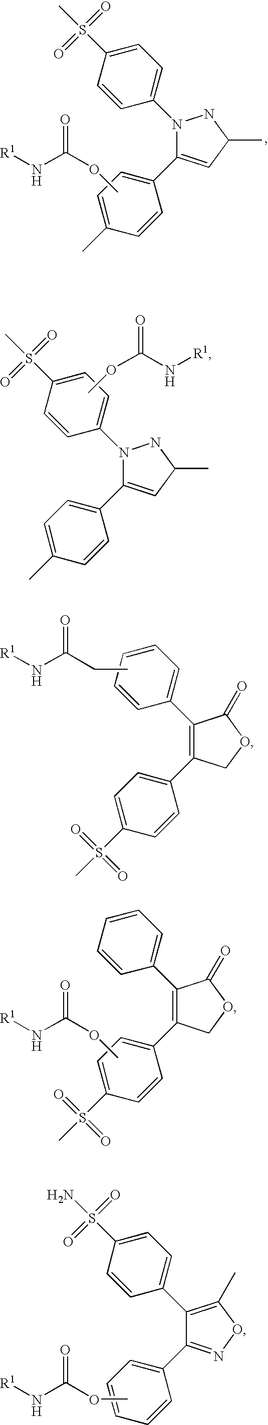

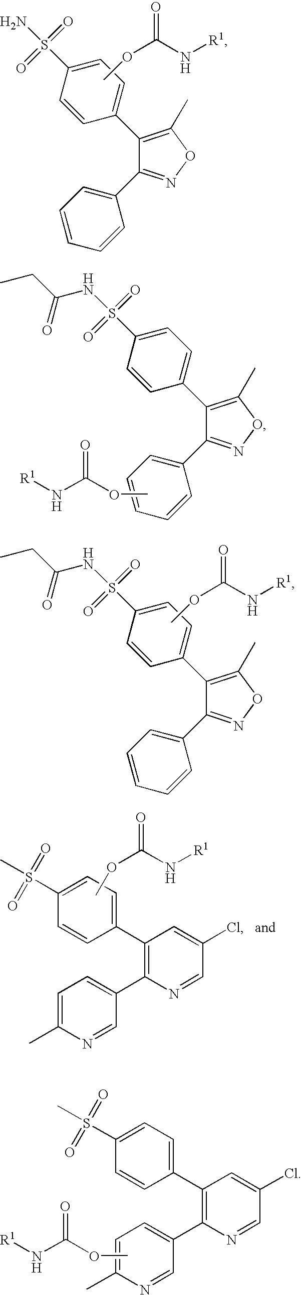

4. The compound of claim 3, wherein the compound of Formula (I) has the structure:

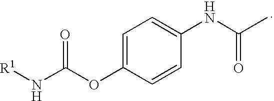

5. The compound of claim 1, wherein R¹ is selected from cyclohexyl and CH₂cyclohexyl.

6. The compound of claim 5 having the structure:

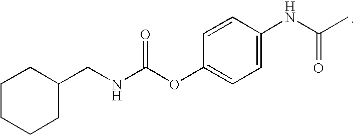

7. The compound of claim 5 having the structure:

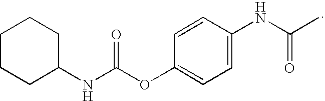

8. The compound of claim 1, wherein the compound, upon inhibition of fatty acid amide hydrolase (FAAH), produces acetaminophen.

9. The compound of claim 8, wherein the inhibition is irreversible inhibition.

10. A pharmaceutical composition comprising a compound, pharmaceutically acceptable salt, pharmaceutically acceptable N-oxide, pharmaceutically active metabolite, pharmaceutically acceptable prodrug, or pharmaceutically acceptable solvate of claim 1, and a pharmaceutically acceptable excipient.

11. A method of treatment comprising administering to a patient having pain, a therapeutically effective amount of a compound, pharmaceutically acceptable salt, pharmaceutically acceptable N-oxide, pharmaceutically active metabolite, pharmaceutically acceptable prodrug, or pharmaceutically acceptable solvate of claim 1.

12. The method of claim 11, wherein the pain is selected from among acute or chronic pain, inflammatory diseases, pain, nociceptive pain, neuropathic pain, inflammatory pain, non-inflammatory pain, painful hemorrhagic cystitis, pain associated with the herpes virus, pain associated with diabetes, peripheral neuropathic pain, central pain, deafferentiation pain, chronic nociceptive pain, stimulus of nociceptive receptors, phantom and transient acute pain, peri-operative pain, cancer pain, pain and spasticity associated with multiple sclerosis, central pain, deafferentiation pain, arachnoiditis, radiculopathies, neuralgias, somatic pain, deep somatic pain, surface pain, visceral pain, acute pain, chronic pain, breakthrough pain, chronic back pain, failed back surgery syndrome, fibromyalgia, post-stroke pain, trigeminal neuralgia, sciatica, pain from radiation therapy, complex regional pain syndromes, causalgia, reflex sympathetic dystrophy, phantom limb pain, myofascial pain, and phantom and transient acute pain.

13. An article of manufacture, comprising a packaging material, and within the packaging material the compound of claim 1 in an amount effective for the treatment of pain, and a label that indicates that the compound is used for the treatment of pain.

14. A process of preparing an ester of an alkylcarbamic acid comprising:
treating an isocyanate of Formula (II)
O—C═N—R¹ Formula (II)
wherein:
R¹ is an optionally substituted group selected from among C₁-C₆ alkyl, C₃-C₉ cycloalkyl, and —C₁-C₄alkyl-(C₃-C₉cycloalkyl);
with a hydroxy-containing compound selected from acetaminophen, propofol, an analgesic agent, an anti-inflammatory agent, an anti-pyretic agent, an NSAID, a metabolite of an analgesic agent, a metabolite of an anti-inflammatory agent, a metabolite of an anti-pyretic agent, and an NSAID metabolite.

15. The process of claim 14, wherein the hydroxy-containing compound is acetaminophen.

16. The process of claim 14, wherein R¹ is selected from among cyclohexyl and CH₂cyclohexyl.

17. An ester of an alkylcarbamic acid acid prepared by the process of claim 14.

18. An ester of an alkylcarbamic acid having the structure 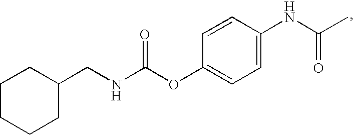
prepared by the process of claim 14.

19. An ester of an alkylcarbamic acid having the structure 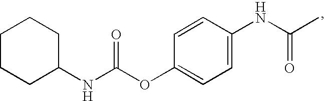
prepared by the process of claim 14.
